(19) United States
(12) Patent Application Publication
(10) Pub. No.: US 2026/0209783 A1
(43) Pub. Date: Jul. 23, 2026

(54) Means and Methods for Increased Protein Expression by Use of a Combination of Transport Proteins and Either Chaperones or Transcription Factors

(71) Applicants: Boehringer Ingelheim RCV GmbH & Co KG, Wien (AT); Validogen GmbH, Raaba-Grambach (AT); Lonza Ltd., Visp (CH)

(72) Inventors: Kristin BAUMANN, Esporles, Balearic Islands (ES); Brigitte GASSER, Wien (AT); Diethard MATTANOVICH, Wien (AT); Roland PRIELHOFER, Leonding (AT); Oezge ATA AKYOL, Wien (AT)

(21) Appl. No.: 19/139,578

(22) PCT Filed: Dec. 15, 2023
(86) PCT No.: PCT/EP2023/086112

§ 371 Date: Jun. 16, 2025

(30) Foreign Application Priority Data
Dec. 16, 2022 (EP) 22214029.5

(51) Int. Cl. C12N 15/81; C07K 14/39; C07K 16/00; C12N 9/18

(52) U.S. Cl. CPC C12N 15/815; C07K 14/39; C07K 16/00; C12N 9/18; C12Y 301/01001; C07K 2317/55; C07K 2317/622

(57) ABSTRACT

The present invention is in the field of recombinant biotechnology, in particular in the field of protein expression. The invention generally relates to a method of increasing the yield and/or titer of at least one protein of interest in a eukaryotic host cell, preferably a yeast, comprising overexpressing in said host cell a) at least one polynucleotide encoding at least one Sbh1 transport protein, and b) (i) at least one polynucleotide encoding at least one Kar2 endoplasmic reticulum helper protein; or (ii) at least one polynucleotide encoding at least one Hac transcription factor. The invention further relates to a recombinant eukaryotic host cell for manufacturing at least one protein of interest, which is engineered to overexpress at least one polynucleotide encoding said at least one Sbh1 transport protein, and b) (i) at least one polynucleotide encoding said at least one Kar2 endoplasmic reticulum helper protein; or (ii) at least one polynucleotide encoding said at least one Hac1 transcription factor as well as the use of said recombinant eukaryotic host cell for manufacturing at least one protein of interest.

### CROSS-REFERENCE TO RELATED APPLICATIONS

The present application claims the benefit of priority of EP Patent Application No. 22 214 029.5 filed 16 Dec. 2022, the content of which is hereby incorporated by reference in its entirety for all purposes.

### FIELD OF THE INVENTION

The present invention is in the field of recombinant biotechnology, in particular in the field of protein expression. The invention generally relates to a method of increasing the yield and/or titer of at least one protein of interest (POI) in a eukaryotic host cell, preferably a yeast, comprising overexpressing in said host cell a) at least one polynucleotide encoding a transport protein, and b) (i) at least one polynucleotide encoding at least one endoplasmic reticulum (ER) helper protein; or (ii) at least one polynucleotide encoding at least one transcription factor. The invention further relates to a recombinant eukaryotic host cell for manufacturing at least one protein of interest as well as the use of said recombinant eukaryotic host cell for manufacturing at least one protein of interest.

### BACKGROUND OF THE INVENTION

Successful production of proteins of interest (POI) has been accomplished both with prokaryotic and eukaryotic hosts. The most prominent examples are bacteria like Escherichia coli, yeasts like Saccharomyces cerevisiae, Pichia pastoris or Hansenula polymorpha, filamentous fungi like Aspergillus awamori or Trichoderma reesei, or mammalian cells like CHO cells. While the yield of some proteins is readily achieved at high rates, many other proteins are only produced at comparatively low levels.

Generally, heterologous protein synthesis may be limited at different levels. Potential limits are transcription and translation, protein folding and, if applicable, secretion, disulfide bridge formation and glycosylation, as well as aggregation and degradation of the target proteins. Transcription can be enhanced by utilizing strong promoters or increasing the copy number of the heterologous gene. However, these measures clearly reach a plateau, indicating that other bottlenecks downstream of transcription limit expression.

High level of protein yield in host cells may also be limited at one or more different steps, like folding, disulfide bond formation, glycosylation, transport within the cell, or release from the cell. Many of the mechanisms involved are still not fully understood and cannot be predicted on the basis of the current knowledge of the state-of-the-art, even when the DNA sequence of the entire genome of a host organism is available. Moreover, the phenotype of cells producing recombinant or endogenous proteins in high yields can be decreased growth rate, decreased biomass formation and overall decreased cell fitness.

Various attempts were made in the art for improving production of a protein of interest, such as overexpressing chaperones which should facilitate protein folding, external supplementation of amino acids, and the like.

However, there is still a need for methods to improve a host cell's capacity to produce and/or secrete proteins of interest. The technical problem underlying the present invention is to comply with this need.

The solution of the technical problem is the provision of means, such as engineered host cells, methods and uses applying said means for increasing the yield and/or titer of at least one protein of interest in a eukaryotic host cell by overexpressing in said host cell at least one polynucleotide encoding at least one transport protein and additionally overexpressing either at least one polynucleotide encoding at least one ER helper protein or at least one polynucleotide encoding at least one transcription factor according to the present invention. These means, methods and uses are described in detail herein, set out in the claims, exemplified in the Examples and illustrated in the Figures.

Accordingly, the present invention provides new methods and uses to increase the yield and/or titer of proteins in host cells which are simple and efficient and suitable for use in industrial methods. The present invention also provides recombinant host cells to achieve this purpose.

It must be noted that as used herein, the singular forms “a”, “an” and “the” include plural references and vice versa unless the context clearly indicates otherwise. Thus, for example, a reference to “a host cell” or “a method” includes one or more of such host cells or methods, respectively, and a reference to “the method” includes equivalent steps and methods that could be modified or substituted known to those of ordinary skill in the art. Similarly, for example, a reference to “methods” or “host cells” includes “a host cell” or “a method”, respectively.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”. For example, A, B and/or C means A, B, C, A+B, A+C, B+C and A+B+C.

The term “about” or “approximately” as used herein means within 20%, preferably within 10%, and more preferably within 5% of a given value or range. It includes also the concrete number, e.g., about 20 includes 20.

The term “less than”, “more than” or “larger than” includes the concrete number. For example, less than 20 means s 20 and more than 20 means 20.

Throughout this specification and the claims or items, unless the context requires otherwise, the word “comprise” and variations such as “comprises” and “comprising” will be understood to imply the inclusion of a stated integer (or step) or group of integers (or steps). It does not exclude any other integer (or step) or group of integers (or steps). When used herein, the term “comprising” can be substituted with “containing”, “composed of”, “including”, “having” or “carrying” and vice versa, by way of example the term “having” can be substituted with the term “comprising”. When used herein, “consisting of” excludes any integer or step not specified in the claim/item. When used herein, “consisting essentially of” does not exclude integers or steps that do not materially affect the basic and novel characteristics of the claim/item.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

It should be understood that this invention is not limited to the particular methodology, protocols, material, reagents, and substances, etc., described herein. The terminologies used herein are for the purpose of describing particular embodiments only and are not intended to limit the scope of the present invention, which is defined solely by the claims/items.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### SUMMARY OF THE INVENTION

The findings of the present inventors are surprising, since overexpression of at least one Sbh1 transport protein as one subunit of the secretion pore in combination with overexpression of either at least one Kar2 endoplasmic reticulum (ER) protein or overexpression of at least one Hac1 transcription factor as described herein was to the best of one's knowledge up to the present invention not brought in connection with increasing the yield and/or titer of at least one protein of interest (POI) in a eukaryotic host cell, particularly in a fungal host cell, preferably in a yeast cell. In sum, the inventors have found that engineering of a certain translocation pore component in combination with a certain chaperone or a particular transcription factor leads to increased translocation of proteins into the endoplasmic reticulum to enter the secretion pathway, thus increasing the yield and/or titer of a POI in a eukaryotic host cell.

In a first aspect, the present invention comprises a method of increasing the yield and/or titer of at least one POI in a eukaryotic host cell which comprises at least one polynucleotide encoding the at least one POI, comprising overexpressing in said host cell:
a) at least one polynucleotide encoding at least one Sbh1 transport protein; and
b) (i) at least one polynucleotide encoding at least one Kar2 ER helper protein; or
   (ii) at least one polynucleotide encoding at least one Hac1 transcription factor, thereby increasing the yield and/or titer of the POI in comparison to a host cell which does not overexpress the polynucleotides of a) and b).

The method of the present invention may comprise:
engineering said host cell to overexpress the polynucleotides of a) and b);
engineering said host cell to comprise at least one polynucleotide encoding the at least one POI, preferably operably linked to a promoter;
culturing said host cell under suitable conditions to overexpress the polynucleotides of a) and b), and to express the at least one polynucleotide encoding the at least one POI, optionally
isolating the at least one POI from the cell culture, and optionally
purifying the at least one POI.

Additionally, the present invention envisages a method of manufacturing at least one POI in a eukaryotic host cell comprising:
providing said host cell engineered to overexpress
a) at least one polynucleotide encoding at least one Sbh1 transport protein, and
b) (i) at least one polynucleotide encoding at least one Kar2 ER helper protein; or
   (ii) at least one polynucleotide encoding at least one Hac1 transcription factor, wherein the host cell further comprises at least one polynucleotide encoding the at least one POI,
culturing said host cell under suitable conditions to overexpress the polynucleotides a) and b) and to express the at least one polynucleotide encoding the at least one POI, optionally
isolating the at least one POI from the cell culture, optionally
purifying the at least one POI, optionally
modifying the at least one POI, and optionally
formulating the at least one POI.

The method of the present invention may comprise that the host cell is engineered to overexpress at least one polynucleotide encoding at least one Hac1 transcription factor, thereby enabling overexpression of the at least one Kar2 ER helper protein.

Further, the present invention may comprise the method of the present invention, wherein the POI is an enzyme, a therapeutic protein, a food additive or a feed additive, preferably wherein the therapeutic protein comprises an antibody or an antibody fragment comprising at least an antigen-binding site, such as an antibody comprising one or more single variable domains.

The present invention may encompass the method of the present invention, wherein the overexpression of said polynucleotides of a) and b) increases the yield and/or titer of at least any one of the model protein Fab (SEQ ID NOs: 55 and 56), scFv (SEQ ID NO: 57), or CES in comparison to a host cell which is not engineered to overexpress said polynucleotides of a) and b).

The present invention may encompass the method of the present invention, wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.

The present invention may envisage the method of the present invention, wherein at least one of the at least one polynucleotide encoding the at least one POI and the polynucleotides of a) and b) are integrated in the genome of the host cell or contained in a vector or plasmid, which does not integrate into the genome, preferably into the chromosome of the host cell.

The present invention may encompass the method of the present invention, wherein the at least one polynucleotide encoding the at least one Sbh1 transport protein and the at least one polynucleotide encoding the at least one Kar2 ER helper protein or the at least one polynucleotide encoding the at least one Hac1 transcription factor encode for the respective proteins according to one of the following:
a) a heterologous Sbh1 transport protein and a heterologous Kar2 ER helper protein or a heterologous Hac1 transcription factor;
b) a homologous Sbh1 transport protein and a homologous Kar2 ER helper protein or a homologous Hac1 transcription factor;
c) a heterologous Sbh1 transport protein and a homologous Kar2 ER helper protein or a homologous Hac1 transcription factor; or
d) a homologous Sbh1 protein transport protein and a heterologous Kar2 ER helper protein or a heterologous Hac1 transcription factor.

The present invention may encompass the method of the present invention, wherein the overexpression of the polynucleotides a) and b) is achieved by
(i) using a recombinant promoter which drives expression of the polynucleotides;
(ii) exchanging or modifying a regulatory sequence operably linked to the polynucleotides; or
(iii) introducing one or more copies of the polynucleotides operably linked to a promoter into the host cell.

The present invention may envisage the method of the present invention, wherein the overexpression of the polynucleotides a) and b) is achieved by
i) exchanging the native promoter operably linked to the endogenous polynucleotides by a stronger recombinant promoter;
ii) exchanging the coding sequence of said endogenous polynucleotides by a codon-optimized coding sequence (such as optimized for mRNA stability or half life or for using the most frequent codons and the like), which codon-optimization is done according to the codon-usage of said host cell;
iii) exchanging a native positive regulatory element of the endogenous polynucleotides for a more efficient positive regulatory element;
iv) introducing a positive regulatory element for the polynucleotides, which element is not present in the respective endogenous expression cassette of the host cell; or
v) deleting a negative regulatory element of the endogenous polynucleotides, which element is normally present in the respective endogenous expression cassette of the host cell, or a combination thereof.

Additionally, the present invention may comprise the method of the present invention, wherein the eukaryotic host cell is particularly a fungal host cell, preferably a yeast host cell, more preferably a yeast host cell selected from the group consisting of Komatagaella, Pichia, Hanselula, Saccharomyces, Kluyveromyces, Yarrowia, Candida and Schizisaccharomyzes, most preferably a yeast host cell selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.

Preferably, said Sbh1 transport protein as used in the methods as defined herein or in the recombinant eukaryotic host cell comprises an amino acid sequence as shown in any one of SEQ ID NOs: 1-9 or a functional homolog thereof comprising at least 90% sequence identity to an amino acid sequence as shown in any one of SEQ ID NO: 1 (Sbh1 K. phaffii), SEQ ID NO: 2 (Sbh1 K. lactis), SEQ ID NO: 3 (Sbh1 Y. lipolytica), SEQ ID NO: 4 (Sbh1 O. polymorpha), SEQ ID NO: 5 (Sbh1 S. cerevisiae), SEQ ID NO: 6 (Sbh2 S. cerevisiae), SEQ ID NO: 7 (Sbh1 A. niger), SEQ ID NO: 8 (Sbh1 T. reesei) or SEQ ID NO: 9 (Sbh1 S. pombe).

Preferably, said Kar2 ER helper protein as used in the methods as defined herein or in the recombinant eukaryotic host cell comprises an amino acid sequence as shown in any one of SEQ ID NOs: 10-18 or a functional homolog thereof comprising at least 90% sequence identity to an amino acid sequence as shown in any one of SEQ ID NO: 10 (Kar2 K. phaffil), SEQ ID NO: 11 (Kar2 K. pastoris), SEQ ID NO: 12 (Kar2 Y. lipolytica), SEQ ID NO: 13 (Kar2 S. cerevisiae), SEQ ID NO: 14 (Kar2 O. polymorpha), SEQ ID NO: 15 (Kar2 T. reesei), SEQ ID NO: 16 (Kar2 C. boidinii), SEQ ID NO: 17 (Kar2 A. niger) or SEQ ID NO: 18 (Kar2 K. lactis).

Preferably, said Hac1 transcription factor as used in the methods as defined herein or in the recombinant eukaryotic host cell comprises at least:
a) a DNA binding domain comprising:
   i) an amino acid sequence as shown in any one of SEQ ID NOs: 19-27, or
   ii) a functional homolog comprising at least 90% sequence identity to an amino acid sequence as shown in any one of SEQ ID NO: 19 (DNA binding domain K. phaffii), SEQ ID NO: 20 (DNA binding domain K. pastoris), SEQ ID NO: 21 (DNA binding domain O. angusta), SEQ ID NO: 22 (DNA binding domain C. boidinii), SEQ ID NO: 23 (DNA binding domain Y. lipolytica), SEQ ID NO: 24 (DNA binding domain K. lactis), SEQ ID NO: 25 (DNA binding domain S. cerevisiae), SEQ ID NO: 26 (DNA binding domain T. reesei) or SEQ ID NO: 27 (DNA binding domain A. niger), and
b) an activation domain.

The present invention may encompass the method of the present invention, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.

Contemplated by the present invention may be the method of the present invention, further comprising overexpressing in said host cell or engineering said host cell to overexpress at least one polynucleotide encoding at least one additional ER helper protein.

Preferably, said at least one additional ER helper protein as used in the methods as defined herein or in the recombinant eukaryotic host cell comprises
(i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37 (Lhs1 K. phaffii), SEQ ID NO: 38 (Lhs1 K. pastoris), SEQ ID NO: 39 (Lhs1 C. boidinii), SEQ ID NO: 40 (Lhs1 O. polymorpha), SEQ ID NO: 41 (Lhs1 S. cerevisiae), SEQ ID NO: 42 (Lhs1 K. lactis), SEQ ID NO: 43 (Lhs1 Y. lipolytica), SEQ ID NO: 44 (Lhs1 A. niger), SEQ ID NO: 45 (Lhs1 T. reesei) or SEQ ID NO: 46 (Lhs1 S. pombe); or
(ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47 (Sil1 K. phaffii), SEQ ID NO: 48 (Sil1 K. pastoris), SEQ ID NO: 49 (Sil1 O. parapolymorpha), SEQ ID NO: 50 (Sil1 S. cerevisiae), SEQ ID NO: 51 (Sil1 K. lactis), SEQ ID NO: 52 (Sil1 C. boidinii), SEQ ID NO: 53 (Sil1 Y. lipolytica) or SEQ ID NO: 54 (Sil1 T. reesei).

In a second aspect, the present invention also comprises a recombinant eukaryotic host cell for manufacturing at least one POI, which is engineered to overexpress
a) at least one polynucleotide encoding at least one Sbh1 transport protein as defined elsewhere herein, and
b) (i) at least one polynucleotide encoding at least one Kar2 ER helper protein as elsewhere herein, or
   (ii) at least one polynucleotide encoding at least one Hac1 transcription factor as elsewhere herein.

The present invention may comprise the recombinant eukaryotic host cell as defined herein, wherein the host cell is engineered to overexpress at least one polynucleotide encoding at least one Hac1 transcription factor, thereby enabling overexpression of the at least one Kar2 ER helper protein.

The present invention may also comprise the recombinant eukaryotic host cell as defined herein, wherein the protein of interest is an enzyme, a therapeutic protein, a food additive or a feed additive, preferably the therapeutic protein comprises an antibody or an antibody fragment comprising at least an antigen-binding site, such as an antibody comprising one or more single variable domains.

The present invention may encompass the recombinant eukaryotic host cell as defined herein, wherein the overexpression of said polynucleotides of a) and b) increases the yield and/or titer of at least any one of the model protein Fab (SEQ ID NOs: 55 and 56), scFv (SEQ ID NO: 57), or carboxylesterase (CES), in comparison to a host cell which is not engineered to overexpress said polynucleotides of a) and b), preferably wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.

The present invention may further comprise the recombinant eukaryotic host cell as defined herein, wherein at least one of the at least one polynucleotide encoding the at least one protein of interest and the polynucleotides of a) and b) are integrated in the genome of the host cell or contained in a vector or plasmid, which does not integrate into the genome of the host cell.

The present invention may also comprise the recombinant eukaryotic host cell as defined herein, wherein overexpression of the polynucleotides is achieved by
(i) using a recombinant promoter which drives expression of the polynucleotides;
(ii) exchanging or modifying a regulatory sequence operably linked to the polynucleotides; or
(iii) introducing one or more copies of the polynucleotides operably linked to a promoter into the host cell. The present invention may also comprise the recombinant eukaryotic host cell as defined herein, wherein overexpression of the polynucleotides is achieved by
i) exchanging the native promoter operably linked to the endogenous polynucleotides by a stronger recombinant promoter;
ii) exchanging the coding sequence of said endogenous polynucleotides by a codon-optimized coding sequence, which codon-optimization is done according to the codon-usage of said host cell;
iii) exchanging a native positive regulatory element of the endogenous polynucleotides for a more efficient positive regulatory element;
iv) introducing a positive regulatory element for the polynucleotides, which element is not present in the respective endogenous expression cassette of the host cell; or
v) deleting a negative regulatory element of the endogenous polynucleotides, which element is normally present in the respective endogenous expression cassette of the host cell, or a combination thereof.

The present invention may further comprise the recombinant eukaryotic host cell as defined herein, wherein the eukaryotic host cell is particularly a fungal host cell, preferably wherein the fungal host cell is a yeast host cell, even more preferably wherein the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.

The present invention may encompass the recombinant eukaryotic host cell as defined herein, wherein the Sbh1 transport protein comprises an amino acid sequence as shown in any one of SEQ ID NOs: 1-9 or a functional homolog thereof comprising at least 90% sequence identity to an amino acid sequence as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

The present invention may also comprise the recombinant eukaryotic host cell as defined herein, wherein the Kar2 ER helper protein comprises an amino acid sequence as shown in any one of SEQ ID NOs: 10-18 or a functional homolog thereof comprising at least 90% sequence identity to an amino acid sequence as shown in any one of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18.

The present invention may comprise the recombinant eukaryotic host cell as defined herein, wherein the Hac1 transcription factor comprises at least:
a) a DNA binding domain comprising:
i) an amino acid sequence as shown in any one of SEQ ID NOs: 19-27, or
ii) a functional homolog thereof comprising at least 90% sequence identity to an amino acid sequence as shown in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27, and
b) an activation domain.

The present invention may further comprise the recombinant eukaryotic host cell as defined herein, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.

The present invention may comprise the recombinant eukaryotic host cell as defined herein, wherein said host cell is additionally engineered to overexpress at least one polynucleotide encoding at least one additional ER helper protein.

The present invention may also comprise the recombinant eukaryotic host cell as defined herein, wherein said at least one additional ER helper protein comprises
(i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46; or
(ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

Contemplated by the present invention is also the use of the recombinant eukaryotic host cell as mentioned above for manufacturing at least one POI.

### BRIEF DESCRIPTION OF THE FIGURES

FIG. 1: (A) Overexpression of sbh1 to increase the yield/titer of Fab and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-Fab.

FIG. 2: (A) Overexpression of sbh1 and Ihs1, but without kar2 to increase the yield/titer of Fab and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-Fab.

FIG. 3: (A) Overexpression of sbh1 and sil1, but without kar2 to increase the yield/titer of Fab and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-Fab.

FIG. 4: (A) Overexpression of sbh1 to increase the yield/titer of scFv and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-scFv.

FIG. 5: (A) Overexpression of sbh1 to increase the yield/titer of CES and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-CES.

FIG. 6: (A) Overexpression of sbh1 and Ihs1, but without kar2 to increase the yield/titer of CES and (B) in combination with kar2 in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-CES.

FIG. 7: Overexpression of sbh1 and hac1(i) to increase the yield/titer of CES in P. pastoris. Results from small scale screenings. The plasmid or plasmids used for engineering the host cell to overexpress these genes or gene combinations are shown below the genes or gene combinations in brackets. The fold-change (FC) values of small scale screenings are an arithmetic mean of up to 20 clones/transformants. All FC are given compared to the unmodified parent strain P_{AOX1}-CES.

FIG. 8: Improvement of Fab secretion (titer and yield) in fed batch bioreactor cultivations. Overview of overexpressed genes ((A) sbh1, kar2; (B) sbh1, kar2, lhs1) or gene combinations ((A) sbh1+kar2 and sbh1+kar2+sil1; (B) sbh1+kar2 and sbh1+lhs1) that increase Fab secretion in P. pastoris in fed batch cultivations. The plasmid or plasmids used for engineering the host cell to overexpress these genes or gene combinations are shown next to the genes or gene combinations in brackets. The fold-change values of fed batch cultivations are those of the single selected clone compared to the unmodified parent strain P_{AOX1}-Fab.

FIG. 9: Improvement of scFv secretion (titer and yield) in fed batch bioreactor cultivations. Overview of overexpressed genes (sbh1, kar2) or gene combinations (sbh1+kar2) that increase scFv secretion in P. pastoris in fed batch cultivations. The plasmid or plasmids used for engineering the host cell to overexpress these genes or gene combinations are shown next to the genes or gene combinations in brackets. The fold-change values of fed batch cultivations are those of the single selected clone unmodified parent strain P_{AOX1}-scFv.

FIG. 10: Improvement of CES secretion (titer and yield) in fed batch bioreactor cultivations. Overview of overexpressed genes (sbh1, kar2) or gene combinations (sbh1+kar2) that increase CES secretion in P. pastoris in fed batch cultivations. The plasmid or plasmids used for engineering the host cell to overexpress these genes or gene combinations are shown next to the genes or gene combinations in brackets. The fold-change values of fed batch cultivations are those of the single selected clone unmodified parent strain P_{AOX1}-CES.

### DETAILED DESCRIPTION OF THE INVENTION

The present invention is based on the surprising finding of the overexpression of at least one polynucleotide encoding at least one Sbh1 transport protein as described herein in combination either with overexpressing at least one polynucleotide encoding at least one Kar2 endoplasmic reticulum (ER) helper protein or with overexpressing at least one polynucleotide encoding at least one Hac1 transcription factor as described herein (which refer to the “proteins of the present invention” herein). Such combination of overexpressed proteins was found to increase the yield and/or titer of at least one protein of interest (POI).

The term “increasing the yield of at least one protein of interest, preferably a recombinant POI, in a host cell” means that the yield of the POI which is expressed by said host cell is increased when compared to the same host cell expressing the same POI under the same culturing conditions, however, without the at least one polynucleotides encoding the at least one Sbh1 transport protein as well as the at least one Kar2 ER helper protein or the at least one Hac1 transcription factor being overexpressed or without being engineered to overexpress the polynucleotides encoding the at least one Sbh1 transport protein as well as the at least one Kar2 ER helper protein or the at least one Hac1 transcription factor.

In this context the term “yield” refers to the amount of POI or model protein(s) as described herein, in particular a Fab, a Fab fragment of the SDZ chimeric antibody (e.g. SEQ ID NOs: 55 and 56), scFV, a monomeric antibody single chain variable fragment (e.g. SEQ ID NO: 57), and/or CES, an enzyme carboxylesterase (see Heinl et al 2010. J Biotechnol 145(2):120-9), respectively, which is/are, for example, harvested from the engineered host cell, and increased yields can be due to increased amounts of production inside the host cell or the increased secretion of the POI by the host cell. The term “yield” also refers to the amount of POI or model protein(s) as described herein per cell/biomass and may be presented by mg POI/g biomass (biomass being measured as dry cell weight (DCW) or wet cell weight (WCW), preferably measured as WCW so that “yield” is present by mg POI/g WCW) of a host cell. The term “titer” when used herein refers similarly to the amount of produced POI or model protein(s) as described herein per volume and may be presented as mg POI/L culture supernatant or whole cell broth. The present invention also comprises a method of increasing the titer of at least one POI in a eukaryotic host cell as defined herein, comprising overexpressing in said host cell said at least one polynucleotide encoding the at least one Sbh1 transport protein as well as overexpressing at least one polynucleotide encoding the at least one Kar2 ER helper protein, or overexpressing at least one polynucleotide encoding the at least one Hac1 transcription factor. An increase in yield can be determined when the yield obtained from an engineered host cell is compared to the yield obtained from a host cell prior to engineering, i.e., from a non-engineered host cell. Preferably, “yield” when used herein in the context of a model protein as described herein, is determined as described in Examples 3, 4, 5 and 6. For example, the term “yield” may refer to the amount of POI that is produced by a certain amount of biomass throughout a submersion cultivation. Therein, the POI can be produced and accumulated inside the cell or be secreted to the culture supernatant. The term “increasing the yield of at least one POI in a host cell” refers to increasing the amount of POI produced within the or by the cell and/or to increasing the amount of POI secreted from the cell.

As will be appreciated by a skilled person in the art, the overexpression of the transport protein either in combination with the ER helper protein or with the transcription factor of the present invention has been shown to increase the yield as well as increase the titer of at least one POI, in particular of a POI. In a preferred embodiment, the yield and/or titer of at least one POI, in particular of at least any one of the model protein Fab (e.g. SEQ ID NOs: 55 and 56), scFv (e.g. SEQ ID NO: 57), or CES is increased when overexpressing the Sbh1 transport protein in combination with Kar2 helper protein or Hac1 transcription factor according to the invention by at least about 1.1-fold, at least about 1.25-fold, at least about 1.5-fold, at least about 2-fold, at least about 3-fold, at least about 4-fold, at least about 5-fold, at least about 7.5-fold, or at least about 10-fold. As used herein, the term “10%, 20%, 30%, 40%, 50%, 60%, 70%, 80%, 90%, 100%, 110%, 120%, 130%, 140%, 150%, 160%, 170%, 180%, 190%, 200%, 300%, 400%, 500%, 600% etc.” refers to “0.1-fold, 0.2-fold, 0.3-fold, 0.4-fold, 0.5-fold, 0.6-fold, 0.7-fold, 0.8-fold, 0.9-fold, 1-fold, 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold etc. Thus, the yield and/or titer of the POI, in particular of the model protein(s) mentioned elsewhere herein may be increased by at least about 10%, at least about 25%, at least about 50%, at least about 100%, at least about 200%, at least about 300%, at least about 400%, at least about 500% or more. The suffix “-fold” refers to multiples. “Onefold” means a whole, “twofold” means twice as much, “threefold” means three times as much. The data as depicted in the Examples and Figures are given as fold change to the host cell prior to engineering, i.e., to a non-engineered host cell.

The term “protein of interest (POI)” as used herein generally relates to any protein (recombinant or endogenous), but preferably relates to a “heterologous protein” or “recombinant protein”. The term “protein of interest” (POI) refers to one polypeptide chain (PPC) (monomere), more than one PPC (dimeres, higher meres), wherein dimers and higher meres comprise more than one PPC of the same polypeptide (homo di- and higher meres) or of different polypeptides (hetero di- and higher meres). Such term also comprises more than one same and different polypeptides (hetero-meres of homo-meres or homo-meres of hetero-meres); or more than one different proteins expressed in one host in one cultivation simultaneously or at different time points, circularly permutated proteins, or reversed proteins. Specific examples of the POI are indicated elsewhere herein and include the model proteins Fab (SEQ ID NOs: 55 and 56), scFv (SEQ ID NO. 57), or CES. As used herein, “recombinant” refers to the alteration of genetic material by human intervention. A recombinant protein can be typically described with reference to how it differs from an endogenous (naturally occurring) counterpart (the “wild-type”). Preferably, the POI expressed by the eukaryotic host cell of the present invention is from a different organism. The POI is preferably not a transport protein, an ER helper protein or a transcription factor, i.e. the transport protein. The ER helper protein(s) and/or the transcription factor as used within the methods and the host cell according to the present invention and the POI are not identical. Such POI as defined herein is expressed by said eukaryotic host cell as defined herein which means that said host cell comprises at least one polynucleotide encoding the at least one POI.

The term “expressing at least one polynucleotide” means when at least one polynucleotide is transcribed to mRNA and the mRNA is translated to a polypeptide. The term “overexpress” generally refers to any amount greater than an expression level exhibited by a reference standard (e.g., the same host cell under the same culturing conditions, which is not engineered to overexpress a polynucleotide encoding a protein). The terms “overexpress,” “overexpressing,” “overexpressed” and “overexpression” in the present invention refer to an expression of a gene product or a polypeptide at a level greater than the expression of the same gene product or polypeptide prior to a genetic alteration of the host cell or in a comparable host which has not been genetically altered at defined conditions. In the present invention, a Sbh1 transport protein comprising an amino acid sequence as shown in any one of SEQ ID NOs: 1-9 or a functional homolog thereof comprising at least 90% sequence identity to an amino acid sequence as shown in any one of SEQ ID NOs: 1-9 is overexpressed in combination with a Kar2 ER helper protein comprising an amino acid sequence as shown in any one of SEQ ID NOs: 10-18 or a functional homolog thereof comprising at least 90% sequence identity to an amino acid sequence as shown in any one of SEQ ID NOs: 10-18; or instead of additionally overexpressing said ER helper protein, a Hac1 transcription factor comprising an amino acid sequence as shown in any one of SEQ ID NOs: 28-36 or a functional homolog thereof comprising at least 90% sequence identity to an amino acid sequence as shown in any one of SEQ ID NOs: 28-36. If a host cell does not comprise a given gene product, it is possible to introduce the gene product into the host cell for expression; in this case, any detectable expression is encompassed by the term “overexpression.” In preferred embodiments, “overexpressing” means “engineering to overexpress” as described below. Such preferred embodiments are contemplated for any embodiment relating to “overexpression” or “overexpressing” as described herein.

A “polynucleotide” as used herein, refers to nucleotides, either ribonucleotides or deoxyribonucleotides or a combination of both, in a polymeric unbranched form of any length. Preferably, a polynucleotide refers to deoxyribonucleotides in a polymeric unbranched form of any length. Here, nucleotides consist of a pentose sugar (deoxyribose), a nitrogenous base (adenine, guanine, cytosine or thymine) and a phosphate group. The terms “polynucleotide(s)”, “nucleic acid sequence(s)” are used interchangeably herein.

As used herein, the term “at least one polynucleotide encoding at least one protein (such as Sbh1, Kar2, Hac1, Sil1, Lhs1)” refers to one polynucleotide encoding one protein (such as Sbh1, Kar2, Hac1, Sil1, Lhs1), two polynucleotides encoding two proteins (two Sbh1, two Kar2, two Hac1, two Sil1, two Lhs1), three polynucleotide encoding three proteins, four polynucleotides encoding four proteins etc. Preferably, one polynucleotide encoding one protein (such as Sbh1, Kar2, Hac1, Sil1, Lhs1) is comprised by the present invention, thereby ending up with an overexpression of one Sbh1 plus one Kar2 or one Sbh1 plus one Hac1 wherein Kar2 is subject to an additional ER helper protein.

As used herein throughout the specification, a “homologue” or “homolog” of the transport proteins as used in the present invention shall mean that a protein has the same or conserved residues at a corresponding position in their primary, secondary or tertiary structure. The term also extends to two or more nucleotide sequences encoding homologous polypeptides. When the function as a transport protein such as Sbh1 is proven with such a homologue, the homologue is called “functional homologue”. A functional homologue performs the same or substantially the same function as the transport protein from which it is derived from. In the case of nucleotide sequences a “functional homologue” preferably means a nucleotide sequence having a sequence different form the original nucleotide sequence, but which still codes for the same amino acid sequence, due to the use of the degenerated genetic code. Additionally functional homologs of a protein according to the invention may be obtained by substituting, deleting, adding, inserting and/or modifying one or more amino acids of/from/to/of the protein, whose substitution(s), deletion(s), additions(s), insertion(s) and/or modification(s) preserve the function of the proteins according to the present invention. Such definition applies mutatis mutandis to the ER helper proteins and the transcription factor according to the invention.

Generally, homologues, can be prepared using any mutagenesis procedure known in the art, such as site-directed mutagenesis, synthetic gene construction, semi-synthetic gene construction, random mutagenesis with screening and/or selection for (functional) homologs, shuffling, etc. Site-directed mutagenesis is a technique in which one or more (e.g., several) mutations are introduced at one or more defined sites in a polynucleotide encoding the parent. Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et ai, 1990, Nucleic Acids Res. 18: 7349-4966. Site-directed mutagenesis can also be accomplished in vivo by methods known in the art. See, e.g., U.S. Patent Application Publication No. 2004/0171 154; Storici et ai, 2001, Nature Biotechnol. 19: 773-776; Kren et ai, 1998, Nat. Med. 4: 285-290; and Calissano and Macino, 1996, Fungal Genet. Newslett. 43: 15-16. Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips. Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al, 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127). Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods known in the art. These methods allow the rapid determination of the importance of individual amino acid residues in a polypeptide. Semi-synthetic gene construction is accomplished by combining aspects of synthetic gene construction, and/or site-directed mutagenesis, and/or random mutagenesis, and/or shuffling. Semisynthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled. Alternatively, homologues for example can be obtained from a natural source such as by screening cDNA libraries of other organisms, or by homology searches in nucleic acid databases.

The function of a homologue can be tested by providing expression cassettes into which the transport protein, the ER helper protein or the transcription factor as defined herein comprising the homologues have been inserted, transforming host cells that carry the sequence encoding a test protein such as one of the model proteins used in the Example section or another POI, and determining the difference in the yield of the model protein or POI under identical conditions.

“Sequence identity” or “% identity” refers to the percentage of residue matches between at least two polypeptides or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. The sequence identity used in the present invention refers to the percentage of having identical amino acids between at least two polypeptide sequences (amino acid sequences). The sequence similarity listed in the present invention refers to the percentage of having similar amino acids being group according to their side chains and charges between at least two polypeptide sequences (amino acid sequences). For purposes of the present invention, the sequence identity between two amino acid sequences or nucleotide sequences is determined using the NCBI BLAST program version 2.2.29 (Jan. 6, 2014) (Altschul et al., Nucleic Acids Res. (1997) 25:3389-3402). Sequence identity of two amino acid sequences can be determined with blastp set at the following parameters: Matrix: BLOSUM62, Word Size: 3; Expect value: 10; Gap cost: Existence=11, Extension=1; Filter=low complexity deactivated; Compositional adjustments: Conditional compositional score matrix adjustment. For purposes of the present invention, the sequence identity between two nucleotide sequences is determined using the NCBI BLAST program version 2.2.29 (Jan. 6, 2014) with blastn set at the following exemplary parameters: Word Size: 28; Expect value: 10; Gap costs: Linear; Filter=low complexity activated; Match/Mismatch Scores: 1, −2. For purposes of the present invention, the sequence identity between two amino acid sequences or nucleotide sequences is further determined using BLAST and EMBOSS Needle algorithm. The sequence identity for the DNA binding domain was assessed by said global pairwise sequence alignment with the EMBOSS Needle algorithm. The EMBOSS Needle webserver (https://www.ebi.ac.uk/Tools/psa/emboss_needle/) was used for pairwise protein sequence alignment using default settings (Matrix: BLOSUM62; Gap open:10; Gap extend: 0.5; End Gap Penalty: false; End Gap Open: 10; End Gap Extend: 0.5). EMBOSS Needle reads two input sequences and writes their optimal global sequence alignment to file. It uses the Needleman-Wunsch alignment algorithm to find the optimum alignment (including gaps) of two sequences along their entire length.

Until now, it was nowhere to be found that the Sbh1 transport protein in combination with the Kar2 helper protein or the Hac1 transcription factor are involved in increasing the yield/titer of a POI, or in general involved in the secretion of a POI by a eukaryotic host cell. Thus, it was surprising that the overexpression of such proteins in a eukaryotic host cell increased the yield/titer of a POI in the present invention.

### Sbh1

The term “transport protein” refers to a protein transport protein that mediates the protein translocation across the ER membrane. In the present invention the at least one transport protein refers to a Sbh1 transport protein which is a component of the heterotrimeric Sec61 complex, which is composed of Ssh1, Sbh1 and Sss1. Thus, Sbh1 refers to one subunit of the secretion pore. Such complex is the major component of a channel-forming translocon complex mediating the protein translocation across the ER. In the present invention the Sbh1 transport protein was originally isolated from Pichia pastoris (Komagataella phaffi) CBS7435 strain (CBS-KNAW culture collection). It is envisioned that the transport protein can be overexpressed over a wide range of host cells. Thus, instead of using the sequences native to the species or the genus, the transport protein sequences may also be taken or derived for overexpression or engineering the host cell to overexpress from other prokaryotic or eukaryotic organisms, preferably from fungal host cells, more preferably from a yeast host cell such as from Komagataella phaffii, Kluyveromyces lactis, Yarrowia lipolytica, Ogataea polymorpha, Saccharomyces cerevisiae, Aspergillus niger, Trichoderma reesei, Schizosaccharomyces pombe. Preferably, the transport protein sequence is taken or derived from Pichia pastoris (Komagataella spp). As used herein, Komagataella spp. comprises all species of the genus Komagataella. In preferred embodiments, the transport protein is derived from Komagataella pastoris, Komagataella pseudopastoris or Komagataella phaffii, most preferably from Komagataella phaffii. The closest homolog from other eukaryotic species may also be taken for the at least one Sbh1 transport protein. The function of such eukaryotic or prokaryotic defined Sbh1 transport proteins is similar in mediating the protein translocation across the ER membrane. In the present invention when addressing “Sbh1 transport protein”, the term “Sbh1” can be used interchangeably.

Preferably, said Sbh1 transport protein of the present invention, being overexpressed in said host cell has an amino acid sequence as shown in any one of SEQ ID NOs: 1-9, or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in any one of SEQ ID NO: 1 (Sbh1 of K. phaffii), SEQ ID NO: 2 (Sbh1 of K. lactis), SEQ ID NO: 3 (Sbh1 of Y. lipolytica), SEQ ID NO: 4 (Sbh1 of O. polymorpha), SEQ ID NO: 5 (Sbh1 of S. cerevisiae), SEQ ID NO: 6 (Sbh2 of S. cerevisiae), SEQ ID NO: 7 (Sbh1 of A. niger), SEQ ID NO: 8 (Sbh1 of T. reesei) or SEQ ID NO: 9 (Sbh1 of S. pombe). In one embodiment said Sbh1 transport protein of the present invention being overexpressed in said host cell as used in the methods, the host cell and the use of the present invention has an amino acid sequence as shown in SEQ ID NO: 1 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 1. In another embodiment said Sbh1 transport protein of the present invention being overexpressed in said host cell as used in the methods, the host cell and the use of the present invention has an amino acid sequence as shown in SEQ ID NO: 2 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 2. In another embodiment said Sbh1 transport protein of the present invention being overexpressed in said host cell as used in the methods, the host cell and the use of the present invention has an amino acid sequence as shown in SEQ ID NO: 3 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 3. In another embodiment said Sbh1 transport protein of the present invention being overexpressed in said host cell as used in the methods, the host cell and the use of the present invention has an amino acid sequence as shown in SEQ ID NO: 4 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 4. In another embodiment said Sbh1 transport protein of the present invention being overexpressed in said host cell as used in the methods, the host cell and the use of the present invention has an amino acid sequence as shown in SEQ ID NO: 5 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 5. In another embodiment said Sbh1 transport protein of the present invention being overexpressed in said host cell as used in the methods, the host cell and the use of the present invention has an amino acid sequence as shown in SEQ ID NO: 6 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 6. In another embodiment said Sbh1 transport protein of the present invention being overexpressed in said host cell as used in the methods, the host cell and the use of the present invention has an amino acid sequence as shown in SEQ ID NO: 7 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 7. In another embodiment said Sbh1 transport protein of the present invention being overexpressed in said host cell as used in the methods, the host cell and the use of the present invention has an amino acid sequence as shown in SEQ ID NO: 8 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 8. In another embodiment said Sbh1 transport protein of the present invention being overexpressed in said host cell as used in the methods, the host cell and the use of the present invention has an amino acid sequence as shown in SEQ ID NO: 9 or a functional homolog thereof having at least 70%, 75%, 80%, 85%, or 90%, such as at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 9. Preferably, the Sbh1 transport protein as used in the methods, the host cell and the used of the present invention has an amino acid sequence as shown in SEQ ID NO: 1 as encoded by SEQ ID NO: 68. Preferably, the Sbh1 transport protein is not identical to the protein of interest as defined herein. Said Sbh1 transport protein of the present invention, being overexpressed in said host cell may also have an amino acid sequence as shown in any one of SEQ ID NOs: 1-9, or a functional homolog thereof having from about 85% to about 95% sequence identity to an amino acid sequence as shown in any one of SEQ ID NO: 1 (Sbh1 of K. phaffii), SEQ ID NO: 2 (Sbh1 of K. lactis), SEQ ID NO: 3 (Sbh1 of Y. lipolytica), SEQ ID NO: 4 (Sbh1 of O. polymorpha), SEQ ID NO: 5 (Sbh1 of S. cerevisiae), SEQ ID NO: 6 (Sbh2 of S. cerevisiae), SEQ ID NO: 7 (Sbh1 of A. niger), SEQ ID NO: 8 (Sbh1 of T. reesei) or SEQ ID NO: 9 (Sbh1 of S. pombe).

In this context, it is important to mention that S. cerevisiae underwent a whole-genome duplication (WGD). This causes S. cerevisiae's genome to have very similar copies of many of its genes. Sbh1 from S. cerevisiae (SEQ ID NO: 5) and Sbh2 from S. cerevisiae (SEQ ID NO: 6) are such a case having the same functionality. Thus, Sbh2 from S. cerevisiae (SEQ ID NO: 6) also falls under the definition of “Sbh1 transport protein”.

### Kar2

The term “ER helper protein” includes a chaperone, a co-chaperone and/or a nucleotide exchange factor. The term “chaperone” as used herein relates to a polypeptide that assist the folding, unfolding, assembly or disassembly of other polypeptides. A chaperone refers to proteins that are involved in the correct folding or unfolding and transportation of newly translated eukaryotic cytosolic and secretory proteins. There are many different families of chaperones, each family acts to aid protein folding in a different way. There are ER chaperones and cytosolic chaperones.

ER chaperones in yeast cells comprise but are not limited to Kar2. Kar2 is involved in protein translocation into ER, binding to unassembled/misfolded ER protein subunits and regulating unfolded protein response (UPR). It interacts with its co-chaperones such as Lhs1, Sil1, Erj5, or others known in the art. Lhs1 and Sil1 also refer to nucleotide exchange factors of Kar2 and also belong to the Hsp70 system (Chang et al., Cell 128 (2007)). In this context, the term “nucleotide exchange factor” refers to a protein that stimulates the exchange (replacement) of nucleoside diphosphates (ADP, GDP) for nucleoside triphosphates (ATP, GTP) bound to other proteins (preferably to chaperones). Erj5, Sec63 and Scj1 belong to the group of Hsp40 type proteins. Erj5 for example is a type I membrane protein with a J domain; required to preserve the folding capacity of the endoplasmic reticulum; loss of the non-essential Erj5 gene leads to a constitutively induced unfolded protein response (Mehnert et al., Molecular biology of the cell, 26 (2014)).

In the present invention the at least one ER helper protein refers to a Kar2 ER helper protein. The additional overexpression of the Kar2 ER helper protein makes sure that the POI is folded correctly in the ER, thereby increasing the yield and/or titer of the POI. In the present invention the Kar2 ER helper protein was originally isolated from Pichia pastoris (Komagataella phaffi) CBS7435 strain (CBS-KNAW culture collection). It is envisioned that the ER helper protein can be overexpressed over a wide range of host cells. Thus, instead of using the sequences native to the species or the genus, the ER helper protein sequences may also be taken or derived for additional overexpression or engineering the host cell to additionally overexpress from other prokaryotic or eukaryotic organisms, preferably from fungal host cells, more preferably from a yeast host cell such from Pichia pastoris (Komagataella pastoris or Komagataella phaffii), Yarrowia lipolytica, Saccharomyces cerevisiae, Ogataea polymorpha, Trichoderma reesei, Candida boidinii, Aspergillus niger, or Kluyveromyces lactis. Preferably, the ER helper protein sequence is taken or derived from Pichia pastoris (Komagataella spp). In preferred embodiments, the ER helper protein is derived from Komagataella pastoris, Komagataella pseudopastoris or Komagataella phaffii, most preferably from Komagataella phaffii. The closest homolog from other eukaryotic species may also be taken for the at least one ER helper protein. The function of such eukaryotic or prokaryotic defined Kar2 ER helper proteins is similar in being involved in protein translocation into ER, binding to unassembled/misfolded ER protein subunits and regulating UPR. In the present invention when addressing “Kar2 ER helper protein”, the term “Kar2” can be used interchangeably.

Preferably, said Kar2 ER helper protein of the present invention, being additionally overexpressed in said host cell has an amino acid sequence as shown in any one of SEQ ID NOs: 10-18, or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in any one of SEQ ID NO: 10 (Kar2 of K. phaffii), SEQ ID NO: 11 (Kar2 of K. pastoris), SEQ ID NO: 12 (Kar2 of Y. lipolytica), SEQ ID NO: 13 (Kar2 of S. cerevisiae), SEQ ID NO: 14 (Kar2 of O. polymorpha), SEQ ID NO: 15 (Kar2 of T. reesei), SEQ ID NO: 16 (Kar2 of C. boidinii), SEQ ID NO: 17 (Kar2 of A. niger) or SEQ ID NO: 18 (Kar2 of K. lactis). In one embodiment said Kar2 ER helper protein of the present invention being overexpressed in said host cell as used in the methods, the host cell and the use of the present invention has an amino acid sequence as shown in SEQ ID NO: 10 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 10. In another embodiment said Kar2 ER helper protein of the present invention being overexpressed in said host cell as used in the methods, the host cell and the use of the present invention has an amino acid sequence as shown in SEQ ID NO: 11 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 11. In another embodiment said Kar2 ER helper protein of the present invention being overexpressed in said host cell as used in the methods, the host cell and the use of the present invention has an amino acid sequence as shown in SEQ ID NO: 12 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 12. In another embodiment said Kar2 ER helper protein of the present invention being overexpressed in said host cell as used in the methods, the host cell and the use of the present invention has an amino acid sequence as shown in SEQ ID NO: 13 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 13. In another embodiment said Kar2 ER helper protein of the present invention being overexpressed in said host cell as used in the methods, the host cell and the use of the present invention has an amino acid sequence as shown in SEQ ID NO: 14 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 14. In another embodiment said Kar2 ER helper protein of the present invention being overexpressed in said host cell as used in the methods, the host cell and the use of the present invention has an amino acid sequence as shown in SEQ ID NO: 15 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 15. In another embodiment said Kar2 ER helper protein of the present invention being overexpressed in said host cell as used in the methods, the host cell and the use of the present invention has an amino acid sequence as shown in SEQ ID NO: 16 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 16. In another embodiment said Kar2 ER helper protein of the present invention being overexpressed in said host cell as used in the methods, the host cell and the use of the present invention has an amino acid sequence as shown in SEQ ID NO: 17 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 17. In another embodiment said Kar2 ER helper protein of the present invention being overexpressed in said host cell as used in the methods, the host cell and the use of the present invention has an amino acid sequence as shown in SEQ ID NO: 18 or a functional homolog thereof having at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 18. Preferably, the Kar2 ER helper protein as used in the methods, host cell and use of the present invention has an amino acid sequence as shown in SEQ ID NO: 10 as encoded by SEQ ID NO: 71. Preferably, the helper protein is not identical to the protein of interest as defined herein Said Kar2 ER helper protein of the present invention, being additionally overexpressed in said host cell may also have an amino acid sequence as shown in any one of SEQ ID NOs: 10-18, or a functional homolog thereof having from about 85% to about 95% sequence identity to an amino acid sequence as shown in any one of SEQ ID NO: 10 (Kar2 of K. phaffii), SEQ ID NO: 11 (Kar2 of K. pastoris), SEQ ID NO: 12 (Kar2 of Y. lipolytica), SEQ ID NO: 13 (Kar2 of S. cerevisiae), SEQ ID NO: 14 (Kar2 of O. polymorpha), SEQ ID NO: 15 (Kar2 of T. reesei), SEQ ID NO: 16 (Kar2 of C. boidinii), SEQ ID NO: 17 (Kar2 of A. niger) or SEQ ID NO: 18 (Kar2 of K. lactis).

The overexpression of said Sbh1 of the present invention and said Kar2 helper protein may increase the yield and/or titer of the protein of interest, such as Fab (SEQ ID NOs: 55 and 56), scFv (SEQ ID NO: 57) and/or of CES, compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the Sbh1 of P. pastoris (K. phaffii) of the present invention and of said ER helper protein Kar2 of P. pastoris (K. phaffii) may increase the yield of the model protein Fab (SEQ ID NOs: 55 and 56) compared to the host cell prior to engineering by at least 120%, 150%, 170%, 190%, 200%, 210%, 220%, 230%, 240%, 250%, 260%, 270%, 280%, 290%, 300%, 350%, 400%, 450%, or 500%. The overexpression of the Sbh1 of P. pastoris (K. phaffii) of the present invention and of said ER helper protein Kar2 of P. pastoris (K. phaffii) may increase the yield of the model protein scFv (SEQ ID NO: 57) to the host cell prior to engineering by at least 150%, 170%, 190%, 200%, 210%, 220%, 230%, 240%, 250%, 260%, 270%, 280%, 290%, 300%, 350%, 400%, or 500%. The overexpression of the Sbh1 of P. pastoris (K. phaffii) of the present invention and of said ER helper protein Kar2 of P. pastoris (K. phaffii) may increase the yield of the model protein CES to the host cell prior to engineering by at least 150%, 200%, 250%, 300%, 350%, 400%, 410%, 420%, 430%, 440%, 450%, 460%, 470%, 480%, 490% or 500%.

### Hac1

The term “transcription factor” refers to a protein that controls the rate of transcription of genetic information from DNA to messenger RNA, by binding to a specific DNA sequence, preferably with its DNA binding domain. Their function is to regulate—and/or activate genes in order to make sure that they are expressed in the right cell at the right time and in the right amount. For example, a transcription factor may initiate the transcription of a specific gene(s) in response to a stimulus, such as starvation or heat shock. In the present invention the at least one transcription factor refers to a Hac1 transcription factor. hac1 encodes a transcription factor of the basic leucine zipper (bZIP) family that is involved in the unfolded protein response (Mori K et al., Genes Cells 1(9):803-17, 1996 and Cox J S and Water P, Cell 87(3):391-404, 1996). Heat stress, drug treatment, mutations in secretory proteins, or overexpression of wild type secretory proteins can cause unfolded proteins to accumulate in the ER, triggering the unfolded protein response (UPR). Hac1 is not essential under normal growth conditions, but is essential under conditions that trigger the UPR. Hac1 binds to a DNA sequence called the UPR element (UPRE) in the promoter of UPR-regulated genes such as kar2, pdi1, eug1, fkb2. The abundance of Hac1 is regulated by splicing of the hac1 mRNA. The spliced hac1 mRNA is translated much more efficiently than the unspliced transcript. Hac1 induces the transcription of genes encoding ER chaperons such as Kar2 for example being involved in the UPR. Increased transcription of genes encoding soluble ER resident proteins, including ER chaperones for example, is a key feature of the UPR. Further, Hac1 increases synthesis of ER-resident proteins required for protein folding.

In the present invention the Hac1 transcription factor was originally isolated from Pichia pastoris (Komagataella phaffi) CBS7435 strain (CBS-KNAW culture collection). It is envisioned that the transcription factor can be overexpressed over a wide range of host cells. Thus, instead of using the sequences native to the species or the genus, the transcription factor sequences may also be taken or derived for additional overexpression or engineering the host cell to additionally overexpress from other prokaryotic or eukaryotic organisms, preferably from fungal host cells, more preferably from a yeast host cell such from Pichia pastoris (Komagataella pastoris or Komagataella phaffii), Ogataea angusta, Candida boidinii, Yarrowia lipolytica, Kluyveromyces lactis, Saccharomyces cerevisiae, Trichoderma reesei, or Aspergillus niger. Preferably, the transcription factor sequence is taken or derived from Pichia pastoris (Komagataella spp). In preferred embodiments, the transcription factor is derived from Komagataella pastoris, Komagataella pseudopastoris or Komagataella phaffii, most preferably from Komagataella phaffii. The closest homolog from other eukaryotic species may also be taken for the at least one transcription factor. The function of such eukaryotic or prokaryotic defined Hac1 transcription factors is similar as it is defined above. In the present invention when addressing “Hac1 transcription factor”, the term “Hac1” can be used interchangeably.

Preferably, said Hac1 transcription factor of the present invention, being additionally overexpressed in said host cell has at least a DNA binding domain comprising an amino acid sequence as shown in any one of SEQ ID NOs: 19-27 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in any one of SEQ ID NO: 19 (DNA binding domain of Hac1 of K. phaffii), SEQ ID NO: 20 (DNA binding domain of Hac1 of K. pastoris), SEQ ID NO: 21 (DNA binding domain of Hac1 of O. angusta), SEQ ID NO: 22 (DNA binding domain of Hac1 of C. boidinii), SEQ ID NO: 23 (DNA binding domain of Hac1 of Y. lipolytica), SEQ ID NO: 24 (DNA binding domain of Hac1 of K. lactis), SEQ ID NO: 25 (DNA binding domain of Hac1 of S. cerevisiae), SEQ ID NO: 26 (DNA binding domain of Hac1 of T. reesei) or SEQ ID NO: 27 (DNA binding domain of Hac1 of A. niger); and any activation domain (e.g.: synthetic, viral or an activation domain of the transcription factor of the present invention or other transcription factors of any species as described elsewhere herein). In one embodiment said Hac1 transcription factor of the present invention being additionally overexpressed in said host cell as used in the methods, the host cell and the use of the present invention has at least a DNA binding domain comprising an amino acid sequence as shown in SEQ ID NO: 19 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 19, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 20 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 20, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 21 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 21, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 22 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 22, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 23 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 23, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 24 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 24, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 25 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 25, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 26 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 26, and any activation domain (e.g.: synthetic, viral or an activation domain of the transcription factor of the present invention or other transcription factors of any species as described elsewhere herein). In another embodiment said Hac1 transcription factor of the present invention being additionally overexpressed in said host cell as used in the methods, the host cell and the use of the present invention has at least a DNA binding domain comprising an amino acid sequence as shown in SEQ ID NO: 27 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 27, and any activation domain (e.g.: synthetic, viral or an activation domain of the transcription factor of the present invention or other transcription factors of any species as described elsewhere herein). Preferably, said Hac1 transcription factor as used within the method, the host cell and the use of the present invention has at least a DNA binding domain comprising an amino acid sequence as shown in SEQ ID NO: 19 or a functional homolog thereof comprising at least 70%, 75%, 80%, 85% or 90% such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in SEQ ID NO: 19, and any activation domain (e.g.: synthetic, viral or an activation domain of the transcription factor of the present invention or other transcription factors of any species as described elsewhere herein). Preferably, the transcription factor is not identical to the protein of interest as defined herein. Said Hac1 transcription factor of the present invention, being additionally overexpressed in said host cell may also have an amino acid sequence as shown in any one of SEQ ID NOs: 19-27, or a functional homolog thereof having from about 85% to about 95% sequence identity to an amino acid sequence as shown in any one of SEQ ID NO: 19 (DNA binding domain of Hac1 of K. phaffii), SEQ ID NO: 20 (DNA binding domain of Hac1 of K. pastoris), SEQ ID NO: 21 (DNA binding domain of Hac1 of O. angusta), SEQ ID NO: 22 (DNA binding domain of Hac1 of C. boidinii), SEQ ID NO: 23 (DNA binding domain of Hac1 of Y. lipolytica), SEQ ID NO: 24 (DNA binding domain of Hac1 of K. lactis), SEQ ID NO: 25 (DNA binding domain of Hac1 of S. cerevisiae), SEQ ID NO: 26 (DNA binding domain of Hac1 of T. reesei) or SEQ ID NO: 27 (DNA binding domain of Hac1 of A. niger).

The arrangement of said DNA binding domain of the transcription factor of the present invention as described herein and any activation domain may be performed according to the skilled person's knowledge and may be performed in any order. The DNA binding domain of the transcription factor of the present invention may be arranged by the skilled person C- or N-terminally, preferably C-terminally.

A “DNA binding domain” as used herein refers to the domain of the transcription factor that binds to DNA of its regulated genes. Preferably, the DNA binding domain of the present invention is selected from the group consisting of SEQ ID NOs. 19, 20, 21, 22, 23, 24, 25, 26 and 27 or a functional homolog thereof comprising at least 90% sequence identity as defined herein to an amino acid sequence as shown in any one of SEQ ID NOs: 19-27. Most preferred is the DNA binding domain as shown in SEQ ID NO. 19.

As used herein, the term “activation domain” refers to any domain capable of activating transcription. As an activation domain each activation domain from any transcription factor of any organism known to the person skilled in the art may be used in the present invention. Preferably, for the transcription factor of the present invention any activation domain of the transcription factor of the present invention of any defined species herein may be used, such as from any one of Pichia pastoris (Komagataella spp), Ogataea angusta, Candida boidinii, Yarrowia lipolytica, Kluyveromyces lactis, Saccharomyces cerevisiae, Trichoderma reesei, or Aspergillus niger, preferably from Pichia pastoris (Komagataella spp). The function of the activation domain can be measured by known methods in the art, i.e. by the yeast-2-Hybrid (Y2H) technique allowing the detection of interacting proteins in living yeast cells.

In a preferred embodiment of the present invention the Hac1 transcription factor refers to an amino acid sequence as shown in any one of SEQ ID NOs. 28-36 or a functional homolog thereof comprising at least 70%, 75%, 80%, 85%, or 90%, such as at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity to an amino acid sequence as shown in any one of SEQ ID NO: 28 (Hac1 of K. phaffii) SEQ ID NO: 29 (Hac1 of K. pastoris), SEQ ID NO: 30 (Hac1 of O. angusta), SEQ ID NO: 31 (Hac1 of C. boidinii), SEQ ID NO: 32 (Hac1 of Y. lipolytica), SEQ ID NO: 33 (Hac1 of K. lactis), SEQ ID NO: 34 (Hac1 of S. cerevisiae), SEQ ID NO: 35 (Hac1 of T. reesei) or SEQ ID NO: 36 (Hac1 of A. niger). Thus, the method, the recombinant host cell and the use of the present invention may further comprise additionally overexpressing a Hac1 transcription factor having an amino acid sequence as shown in SEQ ID NO: 28. In another embodiment, the method, the recombinant host cell and the use of the present invention may further comprise additionally overexpressing a Hac1 transcription factor having an amino acid sequence as shown in SEQ ID NO: 29. In another embodiment, the method, the recombinant host cell and the use of the present invention may further comprise additionally overexpressing a Hac1 transcription factor having an amino acid sequence as shown in SEQ ID NO: 30. In another embodiment, the method, the recombinant host cell and the use of the present invention may further comprise additionally overexpressing a Hac1 transcription factor having an amino acid sequence as shown in SEQ ID NO: 31. In another embodiment, the method, the recombinant host cell and the use of the present invention may further comprise additionally overexpressing a Hac1 transcription factor having an amino acid sequence as shown in SEQ ID NO: 32. In another embodiment, the method, the recombinant host cell and the use of the present invention may further comprise additionally overexpressing a Hac1 transcription factor having an amino acid sequence as shown in SEQ ID NO: 33. In another embodiment, the method, the recombinant host cell and the use of the present invention may further comprise additionally overexpressing a Hac1 transcription factor having an amino acid sequence as shown in SEQ ID NO: 34. In another embodiment, the method, the recombinant host cell and the use of the present invention may further comprise additionally overexpressing a Hac1 transcription factor having an amino acid sequence as shown in SEQ ID NO: 35. In another embodiment, the method, the recombinant host cell and the use of the present invention may further comprise additionally overexpressing a Hac1 transcription factor having an amino acid sequence as shown in SEQ ID NO: 36. Preferably, said Hac1 transcription factor used within the methods, the host cell and the use of the present invention has an amino acid sequence as shown in SEQ ID NO: 28 as encoded by any one of SEQ ID NO: 74, 86, 87 or 88.

The overexpression of said Sbh1 of the present invention and said Hac1 may increase the yield and/or titer of the protein of interest, such as Fab (SEQ ID NOs: 55 and 56), scFv (SEQ ID NO: 57) and/or of CES, compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the Sbh1 of P. pastoris (K. phaffii) of the present invention and of said transcription factor Hac1 of P. pastoris (K. phaffii) may increase the yield of the model protein CES compared to the host cell prior to engineering by at least 120%, 140%, 160%, 180%, 200%, 210%, 220%, 230%, 240%, 250%, 270%, 290%, 300%, 350%, 400%, 450%, or 500%.

Generally, the overexpression of said Hac1 transcription factor which is a transcription factor for chaperones/helper proteins in the ER leads to an overexpression of Kar2 helper protein. Thus, the methods and the host cell of the invention also comprise that the host cell may be engineered to overexpress at least one polynucleotide encoding at least one Hac1 transcription factor which then enables/leads to the overexpression of the at least one Kar2 ER helper protein as defined herein. By engineering the host cell to additionally overexpress (to Sbh1 transport protein) at least one polynucleotide encoding at least one Kar2 ER helper protein with methods described herein and by additionally overexpressing (to Sbh1 protein) at least one polynucleotide encoding at least one Hac1 transcription factor, the same effect is achieved, namely the additional overexpression of at least one Kar2 helper protein, leading to an increased yield and/or titer of the protein of interest due to the overexpression of Sh1 and Kar2 or Sbh1 and Hac1 as defined herein.

The Hac1 transcription factor additionally overexpressed with Sbh1 and used in the methods, in the recombinant host cell and the use of the present invention, does not stimulate the promoter used for expression of the protein of interest, thus not having any effect on the promoter of the POI. It rather has an effect on the promoter of different proteins (such as Kar2) other than the POI.

When introducing the polynucleotide encoding the at least one transport protein under the control of a promoter by a vector or plasmid, the polynucleotide encoding the additional ER helper protein (Kar2 ER helper protein) or the transcription factor (Hac1 transcription factor) may be integrated on the same vector or plasmid under the control of the same promoter or under the control of a different promoter (Sbh1 under the control of one promoter and Kar2 or Hac1 under the control of a different promoter). When introducing the polynucleotide encoding the at least one transport protein under the control of a promoter by a vector or plasmid, the polynucleotide encoding the additional ER helper protein or the additional transcription factor may be integrated simultaneously or consecutively (one after the other) on a different vector or plasmid. If both the polynucleotide encoding the at least one transport protein and the polynucleotide encoding the additional ER helper protein or the additional transcription factor may be introduced on different vectors or plasmids, one plasmid carrying only the at least one transport protein and another plasmid carrying an overexpression cassette for the at least one additional ER helper protein or the at least one additional transcription factor, are preferably used.

When introducing one or more copies of the polynucleotide encoding the at least one transport protein under the control of a promoter by a vector or plasmid, the polynucleotide encoding the additional ER helper protein or the additional transcription factor may be integrated on the same vector or plasmid under the control of the same promoter or under the control of a different promoter (one or more copies of Sbh1 under the control of one promoter and one or more copies of Kar2 or Hac1 under the control of a different promoter). When introducing one or more copies of the polynucleotide encoding the at least one transport protein under the control of a promoter by a vector or plasmid, the polynucleotide encoding the additional ER helper protein or the additional transcription factor may be integrated simultaneously or consecutively (one after the other) on a different vector or plasmid.

The present invention further provides the method of increasing the yield and/or titer of at least one protein of interest in a eukaryotic host cell as defined herein further comprising: i) engineering the host cell to overexpress at least one polynucleotide encoding at least one Sbh1 transport protein as defined herein, and at least one polynucleotide encoding at least one Kar2 ER helper protein or at least one polynucleotide encoding at least one Hac1 transcription factor, ii) engineering said host cell to comprise at least one polynucleotide encoding the at least one protein of interest, iii) culturing said host cell under suitable conditions to overexpress the at least one polynucleotide encoding the at least one Sbh1 transport protein as defined herein, and at least one polynucleotide encoding at least one Kar2 ER helper protein or at least one polynucleotide encoding at least one Hac1 transcription factor, and to express the at least one polynucleotide encoding the at least one protein of interest, optionally iv) isolating the at least one protein of interest from the cell culture, and further optionally v) purifying the at least one protein of interest. It should be noted that the steps recited in (i) and (ii) do not have to be performed in the recited sequence. It is possible to first perform the step recited in (ii) and then (i).

When a host cell is “engineered to overexpress” a given protein, the host cell is manipulated such that the host cell has the capability to express, preferably overexpress the polynucleotides encoding the transport protein (Sbh1), the ER helper proteins (Kar2, Lhs1, Sil1), the transcription factor (Hac1) or functional homologues thereof, thereby expression of a given protein, e.g. POI or model protein is increased compared to the host cell under the same condition prior to manipulation. In one embodiment, “engineered to overexpress” implies that a genetic alteration to a host cell is made in order to increase expression of a protein, i.e. the cell is (intentionally) genetically engineered to overexpress such protein.

“Prior to engineering” or “prior to manipulation” when used in the context of host cells of the present invention means that such host cells are not engineered using polynucleotides encoding the particular proteins of the present invention or functional homologues thereof. Said term thus also means that host cells do not overexpress polynucleotides encoding the proteins of the present invention or functional homologues thereof of or are not engineered to overexpress such polynucleotides encoding the proteins of the invention or functional homologues thereof. Thus a “host cell prior to engineering” or a “host cell prior to manipulation” or a “host cell which does not overexpress the polynucleotides encoding the proteins of the present invention” is a host cell not overexpressing such polynucleotides encoding the proteins of the invention or functional homologues thereof or a host cell not engineered to overexpress such polynucleotides encoding the proteins of the invention or functional homologues thereof. Furthermore, the “host cell prior to engineering” or the “host cell prior to manipulation” or the “host cell which does not overexpress the polynucleotides encoding the proteins of the invention” is the same host cell to which the increase of the yield and/or titer of said protein of interest is compared to but without overexpressing such polynucleotides encoding the proteins of the invention or functional homologues thereof or without being engineered to overexpress such polynucleotides encoding the proteins of the invention or functional homologues thereof.

The term “engineering said host cell to comprise a polynucleotide encoding said at least one protein of interest” as used herein means that a host cell of the present invention is equipped with a polynucleotide encoding at least one protein of interest as defined herein, i.e., a host cell of the present invention is engineered to contain a polynucleotide encoding at least one protein of interest. This can be achieved, e.g., by transformation or transfection or any other suitable technique known in the art for the introduction of a polynucleotide into a host cell.

Procedures used to manipulate polynucleotide sequences, e.g. coding for the proteins of the present invention and/or the POI, the promoters, enhancers, leaders, etc., are well known to persons skilled in the art, e.g. described by J. Sambrook et al., Molecular Cloning: A Laboratory Manual (3rd edition), Cold Spring Harbor Laboratory, Cold Spring Harbor Laboratory Press, New York (2001).

A foreign or target polynucleotide such as the polynucleotides encoding the overexpressed proteins of the invention or POI can be inserted into the chromosome by various means, e.g., by homologous recombination or by using a hybrid recombinase that specifically targets sequences at the integration sites. The foreign or target polynucleotide described above is typically present in a vector (“inserting/integration vector). These vectors are typically circular and linearized before used for homologous recombination. As an alternative, the foreign or target polynucleotides may be DNA fragments joined by fusion PCR or synthetically constructed DNA fragments which are then recombined into the host cell. In addition to the homology arms, the vectors may also contain markers suitable for selection or screening, an origin of replication, and other elements. It is also possible to use heterologous recombination which results in random or non-targeted integration. Heterologous recombination refers to recombination between DNA molecules with significantly different sequences. Methods of recombinations are known in the art and for example described in Boer et al., Appl Microbiol Biotechnol (2007) 77:513-523. One may also refer to Principles of Gene Manipulation and Genomics by Primrose and Twyman (7^{th} edition, Blackwell Publishing 2006) for genetic manipulation of yeast cells.

Polynucleotides encoding the overexpressed proteins of the invention and/or POI may also be present on an expression vector. Such vectors are known in the art. In expression vectors, a promoter is placed upstream of the gene encoding the heterologous protein and regulates the expression of the gene. Multi-cloning vectors are especially useful due to their multi-cloning site. For expression, a promoter is generally placed upstream of the multi-cloning site. A vector for general integration of the polynucleotide encoding the proteins of the invention and/or the POI may be constructed either by first preparing a DNA construct containing the entire DNA sequence coding for the proteins of the invention and/or the POI and subsequently inserting this construct into a suitable expression vector, or by sequentially inserting DNA fragments containing genetic information for the individual elements, such as the DNA binding domain, the activation domain, followed by ligation. As an alternative to restriction and ligation of fragments, recombination methods based on attachment sites (att) and recombination enzymes may be used to insert DNA sequences into a vector. Such methods are described, for example, by Landy (1989) Ann. Rev. Biochem. 58:913-949; and are known to those of skill in the art.

Host cells according to the present invention can be obtained by introducing a vector or plasmid (such as an expression or integration vector/plasmid as mentioned above) comprising the target polynucleotide sequences into the cells. Techniques for transfecting or transforming eukaryotic cells or transforming prokaryotic cells are well known in the art. These can include lipid vesicle mediated uptake, heat shock mediated uptake, calcium phosphate mediated transfection (calcium phosphate/DNA co-precipitation), viral infection, particularly using modified viruses such as, for example, modified adenoviruses, microinjection and electroporation. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylyne glycol mediated uptake. The DNA can be single or double stranded, linear or circular, relaxed or supercoiled DNA. For various techniques for transfecting mammalian cells, see, for example, Keown et al. (1990) Processes in Enzymology 185:527-537. Throughout the specification, expression or integration vectors/plasmids do not refer to the genome of the host cell and such vectors/plasmids do not integrate into the genome of said host cell as defined herein.

The phrase “culturing said host cell under suitable conditions to overexpress the at least one polynucleotide(s) encoding the proteins of the invention and to express the at least one polynucleotide encoding the at least one protein of interest” refers to maintaining and/or growing eukaryotic host cells under conditions (e.g., but not limited to temperature, aeration, pressure, pH, induction, growth rate, culture medium, nutrients duration of the cultivation, mode of nutrient feed(s) etc.) appropriate or sufficient to obtain production of the desired compound (POI) or to obtain or to overexpress the proteins of the present invention.

A host cell according to the invention obtained by transformation with the gene(s) encoding the proteins of the invention gene(s), and/or the POI gene(s) may preferably first be cultivated at conditions to grow efficiently to a large cell number without the burden of expressing a protein. When the cells are prepared for POI expression, suitable cultivation conditions are selected and optimized to produce the POI.

By way of example, using different promoters and/or copies and/or copy numbers and/or integration sites for the polynucleotides encoding the proteins of the invention and the POI(s), the expression of the polynucleotides encoding the proteins of the invention can be controlled with respect to time point and strength of induction in relation to the expression of the polynucleotides encoding the at least one POI(s). For example, prior to induction of POI expression, the polynucleotides encoding the proteins of the invention may be first expressed. This has the advantage that the the proteins of the invention are already present at the beginning of POI translation. Alternatively, the proteins of the invention and POI(s) can be induced at the same time.

An inducible promoter may be used that becomes activated as soon as an inductive stimulus is applied, to direct transcription of the gene under its control. Under growth conditions with an inductive stimulus, the cells usually grow more slowly than under normal conditions, but since the culture has already grown to a high cell number in the previous stage, the culture system as a whole produces a large amount of the protein. An inductive stimulus is preferably the addition of an appropriate agents (e.g. methanol for the AOX-promoter) or the depletion of an appropriate nutrient (e.g., methionine for the MET3-promoter). Also, the addition of ethanol, methylamine, cadmium or copper as well as heat or an osmotic pressure increasing agent can induce the expression depending on the promotors operably linked to the proteins of the invention and the POI(s).

It is preferred to cultivate the host cell(s) according to the invention in a bioreactor under optimized growth conditions to obtain a cell density of at least 1 g/L, preferably at least 10 g/L cell dry weight, more preferably at least 50 g/L cell dry weight. It is advantageous to achieve such yields of biomolecule production not only on a laboratory scale, but also on a pilot or industrial scale.

According to the present invention, due to overexpression of the at least one proteins of the invention, the POI is obtainable in high yields, even when the biomass is kept low. Thus, a high specific yield, which is measured in mg POI/g dry biomass, may be in the range of 1 to 200, such as 50 to 200, such as 100-200, in the laboratory, pilot and industrial scale is feasible. The specific yield of a production host cell according to the invention preferably provides for an increase of at least 1.1 fold, more preferably at least 1.2 fold, at least 1.3 or at least 1.4 fold, in some cases an increase of more than 2 fold can be shown, when compared to the expression of the product without the overexpression of the proteins of the invention.

The host cell according to the invention may be tested for its expression/secretion capacity or yield by measuring the titer of the protein of interest in the supernatant of the cell culture or the cell homogenate of the cells after cell homogenisation by using standard tests, e.g. ELISA, activity assays, LC, HPLC, Surface Plasmon Resonance (Biacore), Western Blot, capillary electrophoresis (Caliper) or SDS-Page.

Preferably, the host cells are cultivated in a minimal medium with a suitable carbon source, thereby further simplifying the isolation process significantly. By way of example, the minimal medium contains an utilizable carbon source (e.g. glucose, glycerol, ethanol or methanol), salts containing the macro elements (potassium, magnesium, calcium, ammonium, chloride, sulphate, phosphate) and trace elements (copper, iodide, manganese, molybdate, cobalt, zinc, and iron salts, and boric acid).

In the case of yeast cells, the cells may be transformed with one or more of the above-described expression vector(s), mated to form diploid strains, and cultured in conventional nutrient media modified as appropriate for inducing promoters, selecting transformants or amplifying the genes encoding the desired sequences. A number of minimal media suitable for the growth of yeast are known in the art. Any of these media may be supplemented as necessary with salts (such as sodium chloride, calcium, magnesium, and phosphate), buffers (such as HEPES, citric acid and phosphate buffer), nucleosides (such as adenosine and thymidine), antibiotics, trace elements, vitamins, and glucose or an equivalent energy source. Any other necessary supplements may also be included at appropriate concentrations that would be known to those skilled in the art. The culture conditions, such as temperature, pH and the like, are those previously used with the host cell selected for expression and are known to the ordinarily skilled artisan. Cell culture conditions for other type of host cells are also known and can be readily determined by the artisan. Descriptions of culture media for various microorganisms are for example contained in the handbook “Manual of Methods for General Bacteriology” of the American Society for Bacteriology (Washington D.C., USA, 1981).

Host cells can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In some embodiments, cells are cultured in shake flasks or deep well plates. In yet other embodiments, cells are cultured in a bioreactor (e.g., in a bioreactor cultivation process). Cultivation processes include, but are not limited to, batch, fed-batch and continuous methods of cultivation. The terms “batch process” and “batch cultivation” refer to a closed system in which the composition of media, nutrients, supplemental additives and the like is set at the beginning of the cultivation and not subject to alteration during the cultivation; however, attempts may be made to control such factors as pH and oxygen concentration to prevent excess media acidification and/or cell death. The terms “fed-batch process” and “fed-batch cultivation” refer to a batch cultivation with the exception that one or more substrates or supplements are added (e.g., added in increments or continuously) as the cultivation progresses. The terms “continuous process” and “continuous cultivation” refer to a system in which a defined cultivation media is added continuously to a bioreactor and an equal amount of used or “conditioned” media is simultaneously removed, for example, for recovery of the desired product. A variety of such processes has been developed and is well-known in the art.

In some embodiments, host cells are cultured for about 12 to 24 hours, in other embodiments, host cells are cultured for about 24 to 36 hours, about 36 to 48 hours, about 48 to 72 hours, about 72 to 96 hours, about 96 to 120 hours, about 120 to 144 hours, or for a duration greater than 144 hours. In yet other embodiments, culturing is continued for a time sufficient to reach desirable production yields of POI.

The above mentioned methods may further comprise a step of isolating the expressed at least one POI from the cell culture and optionally followed by a step of purifying the at least one POI. If the POI is secreted from the cells, it can be isolated and then purified from the culture medium using state of the art techniques. Secretion of the POI from the cells is generally preferred, since the products are recovered from the culture supernatant rather than from the complex mixture of proteins that results when cells are disrupted to release intracellular proteins. A protease inhibitor, such as phenyl methyl sulfonyl fluoride (PMSF) may be useful to inhibit proteolytic degradation during purification, and antibiotics may be included to prevent the growth of adventitious contaminants. The composition may be concentrated, filtered, dialyzed, etc., using methods known in the art. The cell culture after fermentation/cultivation can be centrifuged using a separator or a tube centrifuge to separate the cells from the culture supernatant. The supernatant can then be filtered and concentrated by using a tangential flow filtration. Alternatively, cultured host cells may also be ruptured sonically or mechanically (e.g. high pressure homogenisation), enzymatically or chemically to obtain a cell extract containing the desired POI, from which the POI may be isolated and purified.

Isolation and purification methods for obtaining the POI may be based on methods utilizing difference in solubility, such as salting out, solvent precipitation, heat precipitation, methods utilizing difference in molecular weight, such as size exclusion chromatography, ultrafiltration and gel electrophoresis, methods utilizing difference in electric charge, such as ion-exchange chromatography, methods utilizing specific affinity, such as affinity chromatography, methods utilizing difference in hydrophobicity, such as hydrophobic interaction chromatography and reverse phase high performance liquid chromatography, methods utilizing difference in isoelectric point, such as isoelectric focusing may be used and methods utilizing certain amino acids, such as IMAC (immobilized metal ion affinity chromatography. If the POI is expressed as inactive and soluble Inclusion Bodies the solubilized Inclusion Bodies need to be refolded and may be purified.

The isolated and purified POI can be identified by conventional methods such as Western Blotting or specific assays for POI activity. The structure of the purified POI can be determined by amino acid analysis, amino-terminal peptide sequencing, primary structure analysis for example by mass spectrometry, RP-HPLC, ion exchange-HPLC, ELISA and the like. It is preferred that the POI is obtainable in large amounts and in a high purity level, thus meeting the necessary requirements for being used as an active ingredient in pharmaceutical compositions or as feed or food additive.

The present invention further provides a method of manufacturing at least one protein of interest in a eukaryotic host cell comprising (i) providing the host cell engineered to overexpress at least one polynucleotide encoding at least one Sbh1 transport protein, and either at least one polynucleotide encoding at least one Kar2 ER helper protein or at least one polynucleotide encoding at least one Hac1 transcription factor, wherein the host cell further comprises at least one polynucleotide encoding the at least one protein of interest, (ii) culturing said host cell under suitable conditions to overexpress the polynucleotides encoding the proteins of the invention and to express the at least one polynucleotide encoding the at least one protein of interest, optionally (iii) isolating the at least one protein of interest from the cell culture, optionally (iv) purifying the at least one protein of interest, optionally (v) modifying the at least one protein of interest, and optionally (vi) formulating the at least one protein of interest.

In this context, the term “manufacturing at least one protein of interest in a eukaryotic host cell” as used herein is meant that the protein of interest may be manufactured by using a eukaryotic host cell for the formation of the recombinant host cell. Thereby, the eukaryotic host cell may produce the protein of interest inside the cell and maintain the POI inside the cell (intracellular) or secrete the POI into the culture medium (extracellular), where the host cell is cultured therein. Thus, the POI may be isolated from said culture medium (supernatant of the cell culture) or from the cell homogenate after cell homogenisation.

In this context, the term “modifying the at least one protein of interest” is meant that the POI may be chemically, physically or enzymatically modified. There are many methods known in the art to modify proteins. Proteins can be coupled to carbohydrates or lipids. The POI may be PEGylated (the POI chemically coupled to polyethylenglycole) or HESylated (the POI is chemically coupled to hydroxyethyl starch) for half-life extension. The POI may also be coupled with other moieties such as affinity domains for e.g. human serum albumin for half life extension. The POI also may be treated by a protease or under hydrolytic conditions for cleavage to form the active ingredient from a pre-sequence or to cleave off a tag such as an affinity tag for purification. The POI may also be coupled to other moieties such as toxins, radioactive moieties or any other moiety. The POI may further be treated under conditions to form dimers, trimers and the like.

Additionally, the term “formulating the at least one protein of interest” refers to bringing the POI to conditions, where the POI can be stored for a longer time and/or for optimized pharmaceutical form and/or pharmaceutical application and/or to better adjust the concentration and/or to provide higher concentrations in liquid formulations. Many different methods known in the art are available to stabilize proteins. By exchanging the buffer in which the POI is existent after purification and/or modification, the POI can be brought under conditions, where it is more stable. Different buffer substances and additives, such as sucrose, mild detergents, stabilizer and the like, known in the art can be used. The POI can also be stabilized by lyophilization. For some POIs formulations can be done by formation of complexes of the POI with lipids or lipoproteins, such als polyplexes, and the like. Some protein may be co-formulated with other proteins.

### Protein of Interest

The present invention further comprises that the protein of interest, preferably the recombinant protein of interest, used in the methods, in the recombinant host cell and the use of the present invention may be an enzyme. Preferred enzymes are those which can be used for industrial application, such as in the manufacturing of a detergent, starch, fuel, textile, pulp and paper, oil, personal care products, or such as for baking, organic synthesis, and the like. (see Kirk et al., Current Opinion in Biotechnology (2002) 13:345-351) and/or enzymes used in biotechnology, biopharmaceutical production, biocatalysis and the like, e.g. proteases for cleavage to form the active ingredient from a pre-sequence or to split off a tag such as an affinity tag for purification or an expression and/or solubility enhancing tag or a tag used for qualitative and quantitative analysis of the fusion protein, and the like. In a preferred embodiment, such enzyme as POI refers to CES.

The present invention further comprises that the protein of interest, preferably the recombinant protein of interest, may be a therapeutic protein. A POI may be but is not limited to a protein suitable as a biopharmaceutical substance like an antigen binding protein such as for example an antibody, camelid heavy chain antibody or antibody fragment such as one or more VHH fragment(s) linked together with or without a peptide linker, or antibody derived scaffold, single domain antibodies and derivatives thereof, other not antibody derived affinity scaffolds such as antibody mimetics, growth factor, hormone, vaccine, etc. as described in more detail herein.

Such therapeutic proteins include, but are not limited to, insulin, insulin-like growth factor, hGH, tPA, cytokines, e.g. interleukins such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosisfactor (TNF) TNF alpha and TNF beta, TRAIL; G-CSF, GM-CSF, M-CSF, MCP-1 and VEGF.

Further examples of therapeutic proteins include blood coagulation factors (VII, VIII, IX), alkaline protease from Fusarium, calcitonin, CD4 receptor darbepoetin, DNase (cystic fibrosis), erythropoetin, eutropin (human growth hormone derivative), follicle stimulating hormone (follitropin), gelatin, glucagon, glucocerebrosidase (Gaucher disease), glucosamylase from A. niger, glucose oxidase from A. niger, gonadotropin, growth factors (GCSF, GMCSF), growth hormones (somatotropines), hepatitis B vaccine, hirudin, human antibody fragment, human apolipoprotein AI, human calcitonin precursor, human collagenase IV, human epidermal growth factor, human insulin-like growth factor, human interleukin 6, human laminin, human proapolipoprotein AI, human serum albumin, insulin, insulin and muteins, insulin, interferon alpha and muteins, interferon beta, interferon gamma (mutein), interleukin 2, luteinization hormone, monoclonal antibody 5T4, mouse collagen, OP-1 (osteogenic, neuroprotective factor), oprelvekin (interleukin 11-agonist), organophosphohydrolase, PDGF-agonist, phytase, platelet derived growth factor (PDGF), recombinant plasminogen-activator G, staphylokinase, stem cell factor, tetanus toxin fragment C, tissue plasminogen-activator, and tumor necrosis factor (see Schmidt, Appl Microbiol Biotechnol (2004) 65:363-372).

Preferably, the therapeutic protein is an antigen binding protein. More preferably, the therapeutic protein comprises an antibody, an antibody fragment or an antibody mimetic. Even more preferably, the therapeutic protein is an antibody or an antibody fragment as defined herein. Most preferably, such therapeutic protein as POI comprises an antibody or an antibody fragment comprising at least an antigen-binding site such as an antibody comprising one or more single variable domains.

In a preferred embodiment, the therapeutic protein is an antibody fragment as defined herein, such as Fab (SEQ ID NO: 55 which refers to the Fab heavy chain (HC) and SEQ ID NO: 56 refers to the Fab light chain (LC)) or scFv (SEQ ID NO: 57). The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, IgY, etc., from all sources, e.g. human, rodent, rabbit, cow, sheep, pig, dog, other mammals, chicken, other avians, etc., are considered to be “antibodies.” For example, an antibody fragment may include but not limited to Fv (a molecule comprising the VL and VH), single-chain Fv (scFV) (a molecule comprising the VL and VH connected with by peptide linker), Fab, Fab′, F(ab′)₂, single domain antibody (sdAb) (molecules comprising a single variable domain and 3 CDR), and multivalent presentations thereof. The antibody or fragments thereof may be murine, human, humanized or chimeric antibody or fragments thereof. Examples of therapeutic proteins include an antibody, polyclonal antibody, monoclonal antibody, recombinant antibody, antibody fragments, such as Fab′, F(ab′)2, Fv, scFv, di-scFvs, bi-scFvs, tandem scFvs, bispecific tandem scFvs, sdAb, VHH heavy chain, V_{H}, and V_{L}, or human antibody, humanized antibody, chimeric antibody, IgA antibody, IgD antibody, IgE antibody, IgG antibody, IgM antibody, intrabody, diabody, tetrabody, minibody or monobody. An antibody mimetic refers to an organic compound that binds antigens, but that are not structurally related to antibodies. Such an antibody mimetic refers to artificial peptides or proteins having a molar mass of about 3 to 20 kDA, such as affibody molecules, affilins, affimers, affitins, alphabodies, anticalins, avimers, DARPins, monobodies, nanoCLAMPs as known in the prior art.

The protein of interest may further be a food additive. A food additive is a protein used as nutritional, dietary, digestive, supplements, added to food products for human to maintain or improve the safety, freshness, taste, texture, or appearance of food. A “food” means any natural or artificial diet meal or the like or components of such meals intended or suitable for being eaten, taken in, digested, by a human being.

The protein of interest may further be a feed additive. Feed additives are products used in animal nutrition to achieve an effect on the feed itself, on the animals, on food products obtained from the animals consuming the feed additive, or on the environment. For instance, feed additives are used to enhance flavour of feed, to meet the need for certain nutrients or to increase the performance of animals in good health.

Integration into the Genome or Plasmids

Further, at least one of the at least one polynucleotide encoding the at least one protein of interest and the polynucleotides encoding the proteins of the invention (such as Sbh1 and Kar2; Sbh1 and Hac1) used in the methods, in the recombinant host cell and applied in the use of the present invention is/are preferably integrated into the genome of the host cell. The term “genome” generally refers to the whole hereditary information of an organism that is encoded in the DNA (or RNA for certain viral species). Such term does not include any vectors/plasmids, in other words a vector/plasmid is not comprised by the genome according to the present invention. Preferably, said integration is into the chromosome of said host cell.

The polynucleotides encoding the proteins of the invention may be integrated in its natural locus. “Natural locus” means the location on a specific chromosome, where proteins of the invention are located, for example at the natural locus of the gene encoding Sbh1 and at the natural locus of the gene encoding Kar2 of the present invention. However, in another embodiment, the polynucleotides encoding the proteins of the invention are present in the genome of the host cell not at their natural locus, but integrated ectopically. The term “ectopic integration” means the insertion of a nucleic acid into the genome of a microorganism at a site other than its usual chromosomal locus, i.e., predetermined or random integration.

For yeast cells, the polynucleotides encoding the proteins of the invention and/or the polynucleotide encoding the POI may be inserted into a desired locus, such as but not limited to AOX1, GAP, ENO1, TEF, HIS4 (Zamir et al., Proc. NatL Acad. Sci. USA (1981) 78(6):3496-3500), HO (Voth et al. Nucleic Acids Res. 2001 Jun. 15; 29(12): e59), TYR1 (Mirisola et al., Yeast 2007; 24: 761-766), His3, Leu2, Ura3 (Taxis et al., BioTechniques (2006) 40:73-78), Lys2, ADE2, TRP1, GAL1, ADH1, RGI1 or in the ribosomal RNA gene locus.

Alternatively, at least one of the at least one polynucleotide encoding the at least one protein of interest and the polynucleotides encoding the proteins of the invention (such as Sbh1 and Kar2; Sbh1 and Hac1) used in the methods, in the recombinant host cell and applied in the use of the present invention is/are preferably integrated in a plasmid or vector which does not integrate into the genome of the host cell. The terms “plasmid” and “vector” include autonomously replicating nucleotide sequences as well as genome integrating nucleotide sequences. A skilled person is able to employ suitable plasmids or vectors depending on the host cell used.

Preferably, the plasmid is a eukaryotic expression vector, preferably a yeast expression vector.

Plasmids can be used for the transcription of cloned recombinant nucleotide sequences, i.e. of recombinant genes and the translation of their mRNA in a suitable host organism. Plasmids can also be used to integrate a target polynucleotide into the host cell genome by methods known in the art, such as described by J. Sambrook et al., Molecular Cloning: A Laboratory Manual (3rd edition), Cold Spring Harbor Laboratory, Cold Spring Harbor Laboratory Press, New York (2001). A “plasmid” usually comprise an origin for autonomous replication, selectable markers, a number of restriction enzyme cleavage sites, a suitable promoter sequence and a transcription terminator, which components are operably linked together. The polypeptide coding sequence of interest is operably linked to transcriptional and translational regulatory sequences that provide for expression of the polypeptide in the host cells.

A nucleic acid is “operably linked” when it is placed into a functional relationship with another nucleic acid sequence on the same nucleic acid molecule. For example, a promoter is operably linked with a coding sequence of a recombinant gene when it is capable of effecting the expression of that coding sequence.

Large numbers of suitable plasmids or vectors are known to those of skill in the art and many are commercially available. Examples of suitable vectors are provided in Sambrook et al, eds., Molecular Cloning: A Laboratory Manual (2nd Ed.), Vols. 1-3, Cold Spring Harbor Laboratory (1989), and Ausubel et al, eds., Current Protocols in Molecular Biology, John Wiley & Sons, Inc., New York (1997).

Examples of plasmids using yeast as a host include YIp type vector, YEp type vector, YRp type vector, YCp type vector (Yxp vectors are e.g. described in Romanos et al. 1992, Yeast. 8(6):423-488), pGPD-2 (described in Bitter et al., 1984, Gene, 32:263-274), pYES, pAO815, pGAPZ, pGAPZα, pHIL-D2, pHIL-S1, pPIC3.5K, pPIC9K, pPICZ, pPICZα, pPIC3K, pPINK-HC, pPINK-LC (all available from Thermo Fisher Scientific/Invitrogen), pHWO10 (described in Waterham et al., 1997, Gene, 186:37-44), pPZeoR, pPKanR, pPUZZLE and pPUZZLE-derivatives such as pPM2d, pPM2aK21 or pPM2eH21 (described in Stadlmayr et al., 2010, J Biotechnol. 150(4):519-29.; Marx et al. 2009, FEMS Yeast Res. 9(8):1260-70.); GoldenPiCS system (consisting of the backbones BB1, BB2 and BB3aK/BB3eH/BB3rN); pJ-vectors (e.g. pJAN, pJAG, pJAZ and their derivatives; all available from BioGrammatics, Inc), pJexpress-vectors, pD902, pD905, pD915, pD912 and their derivatives, pD12xx, pJ12xx (all available from ATUM/DNA2.0), pRG plasmids (described in Gnugge et al., 2016, Yeast 33:83-98) 2 μm plasmids (described e.g. in Ludwig et al., 1993, Gene 132(1):33-40). Such vectors are known and are for example described in Cregg et al., 2000, Mol Biotechnol. 16(1):23-52 or Ahmad et al. 2014., Appl Microbiol Biotechnol. 98(12):5301-17. Additionally suitable vectors can be readily generated by advanced modular cloning techniques as for example described by Lee et al. 2015, ACS Synth Biol. 4(9):975-986; Agmon et al. 2015, ACS Synth. Biol., 4(7):853-859; or Wagner and Alper, 2016, Fungal Genet Biol. 89:126-136. Additionally, these and other suitable vectors may be also available from Addgene, Cambridge, MA, USA.

For example, a BB1 plasmid of the GoldenPiCS system may be used to introduce the gene fragments of the proteins of the present invention by using specific restriction enzymes (Table 1). The assembled BB1s carrying the respective coding sequence may then further be processed in the GoldenPiCS system to create the required BB3 integration plasmids as described in Prielhofer et al. 2017.

It is possible to construct single plasmids or vectors carrying the genes encoding the proteins of the invention (such as Sbh1 and Kar2; Sbh1 and Hac1) and the POI(s), or two separate plasmids or vectors, one to carry the proteins of the invention genes and the other one the POI genes. In some embodiments, the gene(s) encoding the POI(s) is/are integrated in the genome and the gene encoding the proteins of the inventions are integrated in a plasmid or vector. In some embodiments, the genes encoding the proteins of the invention are integrated in the genome and the gene(s) encoding the POI(s) is/are integrated in a plasmid or vector. In some embodiments, the gene(s) encoding the POI(s) and the genes encoding the proteins of the invention are integrated in the genome. In some embodiments, the gene(s) encoding the POI(s) and the genes encoding the proteins of the invention are integrated in a plasmid or vector. If multiple genes encoding the POI are used, some genes encoding the POI can be integrated in the genome while others can be integrated in the same or different plasmids or vectors. If multiple genes encoding the proteins of the invention are used, some of the genes can be integrated in the genome while others can be integrated in the same or different plasmids or vectors.

In another embodiment, the at least one polynucleotide encoding the at least one Sbh1 transport protein and the at least one polynucleotide encoding the at least one Kar2 ER helper protein or the at least one polynucleotide encoding the at least one Hac1 transcription factor may encode for a heterologous Sbh1 transport protein and a heterologous Kar2 ER helper protein or a heterologous Hac1 transcription factor.

As used herein, the term “heterologous” (also used as exogenous) means derived from a cell or organism (preferably yeast) with a different genomic background or a synthetic sequence. Thus, a “heterologous transport protein and helper protein or transcription factor” is one that originates from a foreign source (or species, e.g. Sbh1 and Kar2 or Hac1 of S. cerevisiae) and is being used in the source (or species e.g. P. pastoris) other than the foreign source or is a synthetic sequence.

Alternatively, the at least one polynucleotide encoding the at least one Sbh1 transport protein and the at least one polynucleotide encoding the at least one Kar2 ER helper protein or the at least one polynucleotide encoding the at least one Hac1 transcription factor may encode for a homologous Sbh1 transport protein and a homologous Kar2 ER helper protein or a homologous Hac1 transcription factor.

The term “homologous” (also used as endogenous) means derived from the same cell or organism with the same genomic background. Thus, a “homologous transport protein and helper protein or transcription factor” is one that originates from the same source (or species, e.g. Sbh1 and Kar2 or Hac1 of P. pastoris) and is being used in the same source (or species e.g. P. pastoris).

Alternatively, the at least one polynucleotide encoding the at least one Sbh1 transport protein and the at least one polynucleotide encoding the at least one Kar2 ER helper protein or the at least one polynucleotide encoding the at least one Hac1 transcription factor may encode for a heterologous Sbh1 transport protein and a homologous Kar2 ER helper protein or a homologous Hac1 transcription factor.

Further alternatively, the at least one polynucleotide encoding the at least one Sbh1 transport protein and the at least one polynucleotide encoding the at least one Kar2 ER helper protein or the at least one polynucleotide encoding the at least one Hac1 transcription factor may encode for a homologous Sbh1 protein transport protein and a heterologous Kar2 ER helper protein or a heterologous Hac1 transcription factor.

In general, overexpression can be achieved in any ways known to a skilled person in the art as will be described later in detail. It can be achieved by increasing transcription/translation of the gene, e.g. by increasing the copy number of the gene or altering or modifying regulatory sequences. For example, overexpression can be achieved by introducing (additionally in the case of overexpression of the homologous proteins of the invention) one or more copies of the polynucleotides encoding the proteins of the invention or a functional homologue thereof operably linked to a regulatory sequence (e.g. a promoter). For example, the gene can be operably linked to a strong constitutive promoter in order to reach high expression levels. Such promoters can be endogenous promoters or recombinant promoters. Alternatively, it is possible to remove regulatory sequences such that expression becomes constitutive. One can substitute the native promoter of a given gene already existing naturally in the genome (chromosome) of the host cell with a recombinant promoter which increases expression of the gene or leads to constitutive expression of the gene.

Furthermore, overexpression can also be achieved by, for example, modifying the chromosomal location of a particular gene, altering nucleic acid sequences adjacent to a particular gene such as a ribosome binding site or transcription terminator, modifying proteins (e.g., regulatory proteins, suppressors, repressors, enhancers, transcriptional activators and the like) involved in transcription of the gene and/or translation of the gene product, or any other conventional means of deregulating expression of a particular gene routine in the art including but not limited to use of antisense nucleic acid molecules, for example, to block expression of repressor proteins or deleting or mutating the gene for a transcriptional factor which normally represses expression of the gene desired to be overexpressed. Prolonging the life and/or the stability of the mRNA may also improve the level of expression. For example, certain terminator regions may be used to extend the half-lives of mRNA (Yamanishi et al., Biosci. Biotechnol. Biochem. (2011) 75:2234 and US 2013/0244243). If multiple copies of genes are included, the genes can either be located in plasmids of variable copy number or in one copy plasmids which comprise variable copies, in integration cassettes, which only integrate once, not being amplified, but comprise more than one copy or integration cassettes comprising one copy which integrate multiple times or just once and then being amplified; or just integrated and amplified in the chromosome. If the host cell does not comprise the gene encoding the proteins of the invention, it is possible to introduce the gene into the host cell for expression. In this case, “overexpression” means expressing the gene product using any methods known to a skilled person in the art.

Those skilled in the art will find relevant instructions in Martin et al. (Bio/Technology 5, 137-146 (1987)), Guerrero et al. (Gene 138, 35-41 (1994)), Tsuchiya and Morinaga (Bio/Technology 6, 428-430 (1988)), Eikmanns et al. (Gene 102, 93-98 (1991)), EP 0 472 869, U.S. Pat. No. 4,601,893, Schwarzer and POhler (Bio/Technology 9, 84-87 (1991)), Reinscheid et al. (Applied and Environmental Microbiology 60, 126-132 (1994)), LaBarre et al. (Journal of Bacteriology 175, 1001-1007 (1993)), WO 96/15246, Malumbres et al. (Gene 134, 15-24 (1993)), JP-A-10-229891, Jensen and Hammer (Biotechnology and Bioengineering 58, 191-195 (1998)) and Makrides (Microbiological Reviews 60, 512-538 (1996)), inter alia, and in well-known textbooks on genetics and molecular biology.

The overexpression of the polynucleotides encoding the proteins of the invention used in the methods, in the recombinant host cell and the use of the present invention is preferably achieved by using any recombinant promoter known to a person skilled which drives expression of said polynucleotides encoding the proteins of the invention. Such recombinant promoter is suitable for (over)expression of an endogenous (homologous) and of an exogenous (heterologous) protein in a eukaryotic host cell. The endogenous/native promoter operably linked to the endogenous (homologous) protein may be replaced with another stronger recombinant promoter in order to reach high expression levels. Such promoter may be inducible or constitutive. Modification and/or replacement of the endogenous promoter may be performed by mutation or homologous recombination using methods known in the art.

Additionally or alternatively, the overexpression of the polynucleotides encoding the proteins of the invention used in the methods, in the recombinant host cell and the use of the present invention is preferably achieved by exchanging or modifying a regulatory sequence operably linked to said polynucleotides encoding the protein of the invention. In this context, a “regulatory sequence (element)” is a segment of a nucleic acid molecule which is capable of increasing or decreasing the expression of specific genes within an organism. A positive regulatory sequence is capable of increasing the expression, whereas a negative regulatory sequence is capable of decreasing the expression. A regulatory sequence (element) includes for example, promoters, enhancers, silencers, polyadenylation signals, transcription terminators (terminator sequence), coding sequences, internal ribosome entry sites (IRES), and the like. A positive regulatory sequence may comprise, but is not limited to, an enhancer. A negative regulatory sequence may comprise, but is not limited to, a silencer.

By “exchanging a regulatory sequence” in this context, it is meant exchanging the native promoter operably linked to the endogenous polynucleotides encoding the proteins of the invention by a stronger recombinant promoter, exchanging the native terminator sequence of said endogenous polynucleotides encoding the proteins of the invention by a more efficient terminator sequence, exchanging the coding sequence of said endogenous polynucleotides encoding the proteins of the invention by a codon-optimized coding sequence, which codon-optimization is done according to the codon-usage of said host cell, and/or exchanging a native positive regulatory element as defined herein such as an enhancer of the endogenous polynucleotides encoding the proteins of the invention for a more efficient positive regulatory element such as a more efficient enhancer.

As used herein in this context, the term “modifying a regulatory sequence” means introduction of another positive regulatory sequence for the polynucleotides encoding the proteins of the invention, which sequence is not present in the respective endogenous expression cassette of the host cell. Alternatively, “modifying a regulatory sequence” refers to a deletion of a negative regulatory sequence of the endogenous polynucleotides, which element is normally present in the respective endogenous expression cassette of the host cell. Endogenous expression cassette means the sequence coding for a protein including its 5′ and 3′ flanking sequences involved in negative or positive regulation of the expression of said protein, such as promoters, terminators, polyadenylation signals, etc. which is present in a cell in nature and which was not artificially generated by man using recombinant gene technology.

Additionally or alternatively, the overexpression of the polynucleotides encoding the proteins of the invention used in the methods, in the recombinant host cell and the use of the present invention is preferably further be achieved by introducing one or more (additionally in the case of overexpression of the homologous proteins of the invention) copies of the polynucleotides encoding the proteins of the invention operably linked to a promoter into the host cell.

The term “promoter” as used herein refers to a region/sequence operably linked to the gene to be expressed that facilitates the transcription of a particular gene. A promoter typically increases the amount of recombinant product expressed from a nucleotide sequence as compared to the amount of the expressed recombinant product when no promoter exists. A promoter from one organism can be utilized to enhance recombinant protein expression from a sequence that originates from another organism. The promoter can be integrated into a host cell chromosome by homologous recombination using methods known in the art (e.g. Datsenko et al, Proc. Natl. Acad. Sci. U.S.A., 97(12): 6640-6645 (2000)). In addition, one promoter element can increase the amount of proteins expressed for multiple sequences attached in tandem. Hence, one promoter element can enhance the expression of one or more recombinant proteins. Promoter activity may be assessed by its transcriptional efficiency. This may be determined directly by measurement of the amount of mRNA transcription from the promoter, e.g. by Northern Blotting, quantitative PCR or indirectly by measurement of the amount of gene product expressed from the promoter.

The promoter could be an “inducible promoter” or “constitutive promoter.” An “inducible promoter” refers to a promoter which can be induced by the presence or absence of certain factors, and “constitutive promoter” refers to a promoter that is active all the time, independent of an inducer, and therefore allows for continuous transcription of its associated gene or genes.

In a preferred embodiment, both the transcription of the nucleotide sequences encoding the proteins of the invention and the POI are each driven by an inducible promoter. In another preferred embodiment, both the transcription of the nucleotide sequences encoding the proteins of the invention and the POI are each driven by a constitutive promoter. In yet another preferred embodiment, the transcription of the nucleotide sequences encoding the proteins of the invention is driven by a constitutive promoter and the transcription of the nucleotide sequence encoding the POI is driven by an inducible promoter. In yet another preferred embodiment, the transcription of the nucleotide sequences encoding the proteins of the invention is driven by an inducible promoter and the transcription of the nucleotide sequence encoding the POI is driven by a constitutive promoter. Suitable promoters for use with yeast host cells can be found by the person skilled in the art (e.g. Mattanovich et al., Methods Mol. Biol. (2012) 824:329-58; Romanos et al, 1992, Yeast 8:423-488). Preferably, the transcription of the nucleotide sequences encoding the proteins of the invention is driven by at least any one of a constitutive glyceraldehyde-3-phosphate dehydrogenase (GAPDH or GAP), a formate dehydrogenase 1 (FDH1), or a POR1 promoter and the transcription of the nucleotide sequence encoding the POI is driven by an inducible alcohol oxidase (AOX) promoter.

### Host Cell

The methods, the recombinant host cell and the use of the present invention use a eukaryotic cell as a host cell. The host cell of the invention is capable of protein expression and optionally protein secretion. Such host cell is applied in the methods of the present invention. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Preferably, the eukaryotic host cell is a fungal host cell. More preferably, the fungal host cell is a yeast host cell. Examples of yeast cells include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces marxianus), the Candida genus (e.g. Candida utilis, Candida boidinii).

In a preferred embodiment, the genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and re-named to Komagataella spp which comprises Komagataella pastoris, Komagataella phaffii and Komagataella pseudopastoris. Therefore Pichia pastoris is a synonymous for Komagataella pastoris, Komagataella phaffii and Komagataella pseudopastoris.

Examples for Pichia pastoris strains useful in the present invention are X33 and its subtypes GS115, KM71, KM71H; CBS7435 (mut+) and its subtypes CBS7435 mut^{s}, CBS7435 mut^{s}ΔArg, CBS7435 mut^{s}ΔHis, CBS7435 mut^{s}ΔArgAHis, CBS7435 mut^{s} PDI+, CBS704 (=NRRL Y-1603=DSMZ 70382), CBS2612 (=NRRL Y-7556), CBS9173-9189 and DSMZ 70877 as well as mutants thereof. These yeast strains are available from industrial suppliers or cell repositories such as the American Tissue Culture Collection (ATCC), the “Deutsche Sammlung von Mikroorganismen und Zellkulturen” (DSMZ) in Braunschweig, Germany, or from the Dutch “Centraalbureau voor Schimmelcultures” (CBS) in Uetrecht, The Netherlands.

According to a further preferred embodiment, the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe. Mostly preferred is Komagataella phaffii as yeast host cell in the present invention.

### Additional ER Helper Proteins

The methods, the recombinant host cell and the use of the present invention may further comprise additionally overexpressing in said host cell or engineering said host cell to overexpress at least one polynucleotide encoding at least one additional ER helper protein. By further overexpressing in said host cell at least one polynucleotide encoding at least one additional ER helper protein, the yield and/or titer of the protein of interest increases in comparison to a host cell engineered to overexpress the polynucleotides encoding the proteins of the invention (Sbh1 and Kar2 or Hac1) but not engineered to overexpress the at least one polynucleotide encoding the at least one additional ER helper protein.

Preferably, the additional ER helper protein refers to a Lhs1 ER helper protein. In the present invention the Lhs1 ER helper protein was originally isolated from Pichia pastoris (Komagataella phaffi) CBS7435 strain (CBS-KNAW culture collection). It is envisioned that the ER helper protein can be overexpressed over a wide range of host cells. Thus, instead of using the sequences native to the species or the genus, the ER helper protein sequences may also be taken or derived for additional overexpression or engineering the host cell to additionally overexpress from other prokaryotic or eukaryotic organisms, preferably from fungal host cells, more preferably from a yeast host cell such from Pichia pastoris (Komagataella pastoris or Komagataella phaffii), Candida boidinii, Ogataea polymorpha, Saccharomyces cerevisiae, Kluyveromyces lactis, Yarrowia lipolytica, Aspergillus niger, Trichoderma reesei, or Schizosaccharomyces pombe. Preferably, the ER helper protein sequence is taken or derived from Pichia pastoris (Komagataella spp). In preferred embodiments, the ER helper protein is derived from Komagataella pastoris, Komagataella pseudopastoris or Komagataella phaffii, most preferably from Komagataella phaffii. The closest homolog from other eukaryotic species may also be taken for the at least one ER helper protein. Lhs1 and Kar2 reciprocally regulate each other; the ATPase activity of Lhs1 is stimulated by Kar2 and Lhs1 enhances the rate of Kar2 ATP turnover by providing specific nucleotide exchange. The function of such eukaryotic or prokaryotic defined Lhs1 ER helper proteins is similar in being involved in protein translocation into ER, binding to unassembled/misfolded ER protein subunits and regulating UPR. In the present invention when addressing “Lhs1 ER helper protein”, the term “Lhs1” can be used interchangeably.

Preferably, said Lhs1 ER helper protein of the present invention, being additionally overexpressed in said host cell has an amino acid sequence as shown in any one of SEQ ID NOs: 37-46, or a functional homolog thereof having at least 90%, such as at least 91%, 92%, 93%, 94%, 95%, 96%, 97%, 98%, 99% or even 100% sequence identity to an amino acid sequence as shown in any one of SEQ ID NO: 37 (Lhs1 of K. phaffii), SEQ ID NO: 38 (Lhs1 of K. pastoris), SEQ ID NO: 39 (Lhs1 of C. boidinii), SEQ ID NO: 40 (Lhs1 of O. polymorpha), SEQ ID NO: 41 (Lhs1 of S. cerevisiae), SEQ ID NO: 42 (Lhs1 of K. lactis), SEQ ID NO: 43 (Lhs1 of Y. lipolytica), SEQ ID NO: 44 (Lhs1 of A. niger), SEQ ID NO: 45 (Lhs1 of T. reesei) or SEQ ID NO: 46 (Lhs1 of S. pombe). In one embodiment said Lhs1 ER helper protein of the present invention being additionally overexpressed in said host cell as used in the methods, the host cell and the use of the present invention has an amino acid sequence as shown in SEQ ID NO: 37 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 37. In another embodiment said Lhs1 ER helper protein of the present invention being additionally overexpressed in said host cell as used in the methods, the host cell and the use of the present invention has an amino acid sequence as shown in SEQ ID NO: 38 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 38. In another embodiment said Lhs1 ER helper protein of the present invention being additionally overexpressed in said host cell as used in the methods, the host cell and the use of the present invention has an amino acid sequence as shown in SEQ ID NO: 39 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 39. In another embodiment said Lhs1 ER helper protein of the present invention being additionally overexpressed in said host cell as used in the methods, the host cell and the use of the present invention has an amino acid sequence as shown in SEQ ID NO: 40 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 40. In another embodiment said Lhs1 ER helper protein of the present invention being additionally overexpressed in said host cell as used in the methods, the host cell and the use of the present invention has an amino acid sequence as shown in SEQ ID NO: 41 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 41. In another embodiment said Lhs1 ER helper protein of the present invention being additionally overexpressed in said host cell as used in the methods, the host cell and the use of the present invention has an amino acid sequence as shown in SEQ ID NO: 42 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 42. In another embodiment said Lhs1 ER helper protein of the present invention being additionally overexpressed in said host cell as used in the methods, the host cell and the use of the present invention has an amino acid sequence as shown in SEQ ID NO: 43 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 43. In another embodiment said Lhs1 ER helper protein of the present invention being additionally overexpressed in said host cell as used in the methods, the host cell and the use of the present invention has an amino acid sequence as shown in SEQ ID NO: 44 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 44. In another embodiment said Lhs1 ER helper protein of the present invention being additionally overexpressed in said host cell as used in the methods, the host cell and the use of the present invention has an amino acid sequence as shown in SEQ ID NO: 45 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 45. In another embodiment said Lhs1 ER helper protein of the present invention being additionally overexpressed in said host cell as used in the methods, the host cell and the use of the present invention has an amino acid sequence as shown in SEQ ID NO: 46 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 46. Preferably, said Lhs1 ER helper protein of the present invention being additionally overexpressed in said host cell as used in the methods, the host cell and the use of the present invention has an amino acid sequence as shown in SEQ ID NO: 37 as encoded by SEQ ID NO: 77.

The overexpression of said proteins of the invention (Sbh1 and Kar2 or Hac1) and said Lhs1 helper protein may increase the yield and/or titer of the protein of interest, such as Fab (SEQ ID NOs: 55 and 56), scFv (SEQ ID NO: 57) and/or of CES compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the Sbh1 of P. pastoris (K. phaffii) of the present invention and of said ER helper protein Kar2 of P. pastoris (K. phaffii) and of said helper protein Lhs1 of P. pastoris (K. phaffii) may increase the yield of the model protein Fab (SEQ ID NOs: 55 and 56) compared to the host cell prior to engineering by at least 140%, 160%, 180%, 200%, 220%, 240%, 260%, 280%, 290%, 300%, 350%, 400%, 450%, or 500%. The overexpression of the Sbh1 of P. pastoris (K. phaffii) of the present invention and of said ER helper protein Kar2 of P. pastoris (K. phaffii) and of said helper protein Lhs1 of P. pastoris (K. phaffii) may increase the yield of the model protein scFv (SEQ ID NO: 57) compared to the host cell prior to engineering by at least 10%, 20%, 30%, 40%, 50%, 60%, 70%, 80%, 90%, 100%, 120%, 140%, 160%, 180%, 200%, 210%, 220%, 230%, 240%, 260%, 280%, 290%, 300%, 350%, 400%, 450%, or 500%. The overexpression of the Sbh1 of P. pastoris (K. phaffii) of the present invention and of said ER helper protein Kar2 of P. pastoris (K. phaffii) and of said helper protein Lhs1 of P. pastoris (K. phaffii) may increase the yield of the model protein CES compared to the host cell prior to engineering by at least 400%, 500%, 600%, 700%, 800%, 850%, 900%, or 1000%.

Alternatively to the additional overexpression of Lhs1, the additional ER helper protein may refer to a Sil1 ER helper protein. In the present invention the Sil1 ER helper protein was originally isolated from Pichia pastoris (Komagataella phaffi) CBS7435 strain (CBS-KNAW culture collection). It is envisioned that the ER helper protein can be overexpressed over a wide range of host cells. Thus, instead of using the sequences native to the species or the genus, the ER helper protein sequences may also be taken or derived for additional overexpression or engineering the host cell to additionally overexpress from other prokaryotic or eukaryotic organisms, preferably from fungal host cells, more preferably from a yeast host cell such from Pichia pastoris (Komagataella pastoris or Komagataella phaffii), Ogataea parapolymorpha, Saccharomyces cerevisiae, Kluyveromyces lactis, Candida boidinii, Yarrowia lipolytica, or Trichoderma reesei. Preferably, the ER helper protein sequence is taken or derived from Pichia pastoris (Komagataella spp). In preferred embodiments, the ER helper protein is derived from Komagataella pastoris, Komagataella pseudopastoris or Komagataella phaffii, most preferably from Komagataella phaffii. The closest homolog from other eukaryotic species may also be taken for the at least one ER helper protein. Sil1 also interacts with the ATPase domain of Kar2. The function of such eukaryotic or prokaryotic defined Sil1 ER helper proteins is similar in being involved in protein translocation into ER, binding to unassembled/misfolded ER protein subunits and regulating UPR. In the present invention when addressing “Sil1 ER helper protein”, the term “Sil1” can be used interchangeably.

Preferably, said Sil1 ER helper protein of the present invention, being additionally overexpressed in said host cell has an amino acid sequence as shown in any one of SEQ ID NOs: 47-54, or a functional homolog thereof having at least 90%, such as at least 91%, 92%, 93%, 94%, 95%, 96%, 97%, 98%, 99% or even 100% sequence identity to an amino acid sequence as shown in any one of SEQ ID NO: 47 (Sil1 of K. phaffii), SEQ ID NO: 48 (Sil1 of K. pastoris), SEQ ID NO: 49 (Sil1 of O. parapolymorpha), SEQ ID NO: 50 (Sil1 of S. cerevisiae), SEQ ID NO: 51 (Sil1 of K. lactis), SEQ ID NO: 52 (Sil1 of C. boidinii), SEQ ID NO: 53 (Sil1 of Y. lipolytica), or SEQ ID NO: 54 (Sil1 of T. reesei). In one embodiment said Sil1 ER helper protein of the present invention being additionally overexpressed in said host cell as used in the methods, the host cell and the use of the present invention has an amino acid sequence as shown in SEQ ID NO: 47 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 47. In another embodiment said Sil1 ER helper protein of the present invention being additionally overexpressed in said host cell as used in the methods, the host cell and the use of the present invention has an amino acid sequence as shown in SEQ ID NO: 48 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 48. In another embodiment said Sil1 ER helper protein of the present invention being additionally overexpressed in said host cell as used in the methods, the host cell and the use of the present invention has an amino acid sequence as shown in SEQ ID NO: 49 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 49. In another embodiment said Sil1 ER helper protein of the present invention being additionally overexpressed in said host cell as used in the methods, the host cell and the use of the present invention has an amino acid sequence as shown in SEQ ID NO: 50 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 50. In another embodiment said Sil1 ER helper protein of the present invention being additionally overexpressed in said host cell as used in the methods, the host cell and the use of the present invention has an amino acid sequence as shown in SEQ ID NO: 51 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 51. In another embodiment said Sil1 ER helper protein of the present invention being additionally overexpressed in said host cell as used in the methods, the host cell and the use of the present invention has an amino acid sequence as shown in SEQ ID NO: 52 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 52. In another embodiment said Sil1 ER helper protein of the present invention being additionally overexpressed in said host cell as used in the methods, the host cell and the use of the present invention has an amino acid sequence as shown in SEQ ID NO: 53 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 53. In another embodiment said Sil1 ER helper protein of the present invention being additionally overexpressed in said host cell as used in the methods, the host cell and the use of the present invention has an amino acid sequence as shown in SEQ ID NO: 54 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 54. Preferably, said Sil1 ER helper protein of the present invention being additionally overexpressed in said host cell as used in the methods, the host cell and the use of the present invention has an amino acid sequence as shown in SEQ ID NO: 47 as encoded by SEQ ID NO: 80.

The overexpression of said proteins of the invention (Sbh1 and Kar2 or Hac1) and said Sil1 helper protein may increase the yield and/or titer of the protein of interest, such as Fab (SEQ ID NOs: 55 and 56), scFv (SEQ ID NO: 57) and/or of CES, compared to the host cell prior to engineering by at least 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500%. The overexpression of the Sbh1 of P. pastoris (K. phaffii) of the present invention and of said ER helper protein Kar2 of P. pastoris (K. phaffii) and of said helper protein Sil1 of P. pastoris (K. phaffii) may increase the yield of the model protein Fab (SEQ ID NOs: 55 and 56) compared to the host cell prior to engineering by at least 160%, 170%, 180%, 190% 200%, 220%, 240%, 260%, 280%, 300%, 350%, 400%, 450%, or 500%. The overexpression of the Sbh1 of P. pastoris (K. phaffii) of the present invention and of said ER helper protein Kar2 of P. pastoris (K. phaffii) and of said helper protein Sil1 of P. pastoris (K. phaffii) may increase the yield of the model protein scFv (SEQ ID NO: 57) compared to the host cell prior to engineering by at least 10%, 20%, 30%, 40%, 50%, 60%, 70%, 80%, 90%, 100%, 120%, 140%, 160%, 180%, 200%, 220%, 240%, 260%, 280%, 300%, 350%, 400%, 450%, or 500%. The overexpression of the Sbh1 of P. pastoris (K. phaffii) of the present invention and of said ER helper protein Kar2 of P. pastoris (K. phaffii) and of said helper protein Sil1 of P. pastoris (K. phaffii) may increase the yield of the model protein CES compared to the host cell prior to engineering by at least 10%, 20%, 30%, 40%, 50%, 60%, 70%, 80%, 90%, 100%, 120%, 130%, 140%, 160%, 180%, 200%, 220%, 240%, 260%, 280%, 300%, 350%, 400%, 450%, or 500%.

When introducing the polynucleotides encoding the proteins of the invention under the control of a promoter by a vector or plasmid, the polynucleotide encoding the additional ER helper protein (Lhs1 or Sil1) are integrated on the same vector or plasmid under the control of the same promoter or under the control of different promoters, e.g.: (a) Sbh1 under the control of one promoter, Kar2 or Hac1 under the control of a different promoter and Lhs1 or Sil1 under the control of another different promoter or b) Sbh1 and Kar2 or Hac1 under the control of the same promoter and Lhs1 or Sil1 under the control of a different promoter or c) Sbh1 under the control of one promoter and Kar2 or Hac1 and Lhs1 or Sil1 under the control of another promoter. When introducing the polynucleotides encoding the proteins of the invention under the control of a promoter by a vector or plasmid, the polynucleotide encoding the additional ER helper protein (Lhs1 or Sil1) are integrated simultaneously or consecutively (one after the other) on a separate vector or plasmid (one vector/plasmid comprising the polynucleotides encoding the proteins of the invention, another vector/plasmid comprising the polynucleotide encoding the ER helper protein).

When introducing one or more copies of the polynucleotides encoding the proteins of the invention under the control of a promoter by a vector or plasmid, the polynucleotide encoding the one or more copies of the additional ER helper protein is integrated on the same vector or plasmid under the control of the same promoter or under the control of different promoters. When introducing one or more copies of the polynucleotides encoding the proteins of the invention under the control of a promoter by a vector or plasmid, the one or more copies of the polynucleotide encoding the additional ER helper protein are integrated simultaneously or consecutively (one after the other) on another different vector or plasmid.

The overexpression of the additional ER helper protein (Lhs1 or Sil1 in combination with the proteins of the invention) may make sure that the POI is folded correctly in the ER, thereby increasing the yield/titer of the POI even more. The additional ER helper protein (Lhs1 or Sil1) interacts as a co-chaperone with the ER helper protein Kar2 when folding the POI.

The overexpression of or the engineering of the host cell to overexpress said additional ER helper protein (Lhs1 or Sil1) is achieved in any ways known to a skilled person in the art as it is also described herein previously for the proteins of the present invention.

Additionally to the overexpression of Lhs1 or Sil1 in combination with the proteins of the invention, the present invention may also comprise another overexpression of another second ER helper protein, which may refer to Erj5. The Erj5 ER helper protein was originally isolated from Pichia pastoris (Komagataella phaffi) CBS7435 strain (CBS-KNAW culture collection). It is envisioned that the ER helper protein can be overexpressed over a wide range of host cells. Thus, instead of using the sequences native to the species or the genus, the ER helper protein sequences may also be taken or derived for additional overexpression or engineering of the host cell to additionally overexpress from other prokaryotic or eukaryotic organisms, preferably from fungal host cells, more preferably from a yeast host cell such from Pichia pastoris (Komagataella pastoris or Komagataella phaffii), Ogataea parapolymorpha, Candida boidinii, Kluyveromyces lactis, Aspergillus niger, Trichoderma reesei, Saccharomyces cerevisiae, Yarrowia lipolytica, or Schizosaccharomyces pombe. Preferably, the ER helper protein sequence is taken or derived from Pichia pastoris (Komagataella spp). In preferred embodiments, the ER helper protein is derived from Komagataella pastoris, Komagataella pseudopastoris or Komagataella phaffii, most preferably from Komagataella phaffii. In the present invention when addressing “Erj5 ER helper protein”, the term “Erj5” can be used interchangeably.

Preferably, said Erj5 ER helper protein of the present invention, being additionally overexpressed in said host cell has an amino acid sequence as shown in any one of SEQ ID NOs: 58-67, or a functional homolog thereof having at least 90%, such as at least 91%, 92%, 93%, 94%, 95%, 96%, 97%, 98%, 99% or even 100% sequence identity to an amino acid sequence as shown in any one of SEQ ID NO: 58 (Erj5 of K. phaffii), SEQ ID NO: 59 (Erj5 of K. pastoris), SEQ ID NO: 60 (Erj5 of O. parapolymorpha), SEQ ID NO: 61 (Erj5 of C. boidinii), SEQ ID NO: 62 (Erj5 of K. lactis), SEQ ID NO: 63 (Erj5 of A. niger), SEQ ID NO: 64 (Erj5 of T. reesei), SEQ ID NO: 65 (Erj5 of S. cerevisiae), SEQ ID NO: 66 (Erj5 of Y. lipolytica), SEQ ID NO: 67 (Erj5 of S. pombe). In one embodiment said Erj5 ER helper protein of the present invention being additionally overexpressed in said host cell as used in the methods, the host cell and the use of the present invention has an amino acid sequence as shown in SEQ ID NO: 58 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 58. In another embodiment said Erj5 ER helper protein of the present invention being additionally overexpressed in said host cell as used in the methods, the host cell and the use of the present invention has an amino acid sequence as shown in SEQ ID NO: 59 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 59. In one embodiment said Erj5 ER helper protein of the present invention being additionally overexpressed in said host cell as used in the methods, the host cell and the use of the present invention has an amino acid sequence as shown in SEQ ID NO: 60 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 60. In one embodiment said Erj5 ER helper protein of the present invention being additionally overexpressed in said host cell as used in the methods, the host cell and the use of the present invention has an amino acid sequence as shown in SEQ ID NO: 61 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 61. In one embodiment said Erj5 ER helper protein of the present invention being additionally overexpressed in said host cell as used in the methods, the host cell and the use of the present invention has an amino acid sequence as shown in SEQ ID NO: 62 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 62. In one embodiment said Erj5 ER helper protein of the present invention being additionally overexpressed in said host cell as used in the methods, the host cell and the use of the present invention has an amino acid sequence as shown in SEQ ID NO: 63 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 63. In one embodiment said Erj5 ER helper protein of the present invention being additionally overexpressed in said host cell as used in the methods, the host cell and the use of the present invention has an amino acid sequence as shown in SEQ ID NO: 64 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 64. In one embodiment said Erj5 ER helper protein of the present invention being additionally overexpressed in said host cell as used in the methods, the host cell and the use of the present invention has an amino acid sequence as shown in SEQ ID NO: 65 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 65. In one embodiment said Erj5 ER helper protein of the present invention being additionally overexpressed in said host cell as used in the methods, the host cell and the use of the present invention has an amino acid sequence as shown in SEQ ID NO: 66 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 66. In one embodiment said Erj5 ER helper protein of the present invention being additionally overexpressed in said host cell as used in the methods, the host cell and the use of the present invention has an amino acid sequence as shown in SEQ ID NO: 67 or a functional homolog thereof having at least 90%, such as at least 91%, 92%, 93%, 94%, 95%, 96%, 97%, 98%, 99% or even 100% sequence identity to an amino acid sequence as shown in SEQ ID NO: 67. Preferably, said Erj5 ER helper protein of the present invention being additionally overexpressed in said host cell as used in the methods, the host cell and the use of the present invention has an amino acid sequence as shown in SEQ ID NO: 58 as encoded by SEQ ID NO: 83.

The present invention also provides a recombinant eukaryotic host cell for manufacturing at least one protein of interest, which is engineered to overexpress at least one polynucleotide encoding at least one Sbh1 transport protein as defined herein, and at least one polynucleotide encoding at least one Kar2 ER helper protein as defined herein, or at least one polynucleotide encoding at least one Hac1 transcription factor as defined herein. The additional overexpression of at least one polynucleotide encoding at least one Lhs1 or Sil1 ER helper protein is also comprised herein when addressing the recombinant eukaryotic host cell. The same applies to the further additional overexpression of at least one polynucleotide encoding at least one Erj5 ER helper protein.

A “recombinant eukaryotic host cell” refers to a eukaryotic host cell as defined throughout herein that has been genetically altered to comprise (a) nucleic acid sequence(s) which was/were not native to said cell.

The present invention further encompasses the use of the recombinant eukaryotic host cell as used herein for manufacturing at least one protein of interest.

All definitions as defined throughout herein are applicable, where necessary, to the recombinant eukaryotic host cell as well as to the use.

### EXAMPLES OF THE INVENTION

The following examples are put forth to provide those of ordinary skill in the art with a complete disclosure and description of how to make and use the subject invention, and are not intended to limit the scope of what is regarded as the invention and defined in the claims. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

The examples below will demonstrate that the newly identified helper protein combination(s) increase(s) the titer (product per volume in mg/L) and the yield (product per biomass in mg/g biomass measured as dry cell weight or wet cell weight), respectively, of recombinant proteins upon its/their overexpression. As an example, the yield of recombinant secretory proteins (monomeric antibody single chain variable fragments scFv, dimeric antibody Fab binding fragment, or enzyme carboxylesterase) in the yeast Pichia pastoris are increased. The positive effect was shown in shaking cultures (conducted in shake flasks or deep well plates) and in lab scale fed-batch cultivations.

### Example 1: Construction and Selection of P. pastoris Strains Secreting Antibody Fragments scFv, Fab and CES

P. pastoris CBS7435 mut^{S} variant (genome sequenced by Sturmberger et al. 2016) was used as host strain. The pPM2d_pGAP and pPM2d_pAOX expression plasmids are derivatives of the pPuzzle_ZeoR plasmid backbone described in WO2008/128701A2, consisting of the pUC19 bacterial origin of replication and the Zeocin antibiotic resistance cassette. Expression of the heterologous gene is mediated by the P. pastoris glyceraldehyde-3-phosphate dehydrogenase (GAP) promoter or alcohol oxidase (AOX) promoter, respectively, and the S. cerevisiae CYC1 transcription terminator. The plasmids already contained the N-terminal S. cerevisiae alpha mating factor pre-pro leader sequence. The genes for the scFv, Fab, and carboxylesterase (CES) were codon-optimized by a commercial supplier (GeneArt/Thermo Fisher or DNA2.0/ATUM) and obtained as synthetic DNA. A His6-tag was fused C-terminally to the scR (scFv) and CES genes for detection. After restriction digest with XhoI and BamHI (for scR), XhoI and SfiI (for CES) or SbfI and SfiI (for Fab heavy (HC) and light (LC) chain genes, which each already contained the N-terminal S. cerevisiae alpha mating factor pre-pro leader sequence), each gene was ligated into both plasmids pPM2d_pGAP and pPM2d_pAOX digested with the respective restriction enzymes. For the Fab, after sequence verification of LC and HC, the expression cassettes for both chains were combined onto one vector by using the compatible restriction enzymes MreI and AgeI. Plasmids were linearized either using AvrlI restriction enzyme (for pPM2d_pGAP), Pmel restriction enzyme (for pPM2d_pAOX) or Bsu361 restriction enzyme (for Fab expression plasmid), respectively, prior to electroporation (using a standard transformation protocol as described in Gasser et al. 2013. Future Microbiol. 8(2):191-208) into P. pastoris. Selection of positive transformants was performed on YPD plates (per liter: 10 g yeast extract, 20 g peptone, 20 g glucose, 20 g agar-agar) containing 100 μg/mL of Zeocin for scR and CES or 50 μg/mL of Zeocin in case of Fab. Colony PCR was used to ensure the presence of the transformed plasmid. Therefore, genomic DNA was obtained by cooking and freezing of P. pastoris colonies for 5 minutes each and directly applied for PCR with the appropriate primers.

Single colonies (in total ~120) of all transformation approaches were picked from transformation plates into single wells of 96-deep well plates. After an initial growth phase to generate biomass, expression from the AOX1 promoter was induced by supplementation with a media formulation containing methanol (4 times in total). After 72 hours from first methanol induction, all deep well plates were centrifuged and supernatants of all wells were harvested into stock microtiter plates for subsequent analysis. Expression from the GAP promoter was continued by supplementation of glucose at defined points of time (i.e. twice per day for 2 days) after the initial growth phase. After a total of 110 hours from the initial inoculation, cultures were harvested as above.

The clones with the highest productivities in small scale screenings (Example 3) and fed batch cultivations (Example 4) were selected to be the basic production strains for further engineering. The clone CBS7435 mut^{S} pAOX scR 4E3 was selected as basic production strain for scFv secretion. The clone CBS7435 mut^{S} pAOX Fab #9 was selected as basic production strain for Fab secretion. The clone CBS7435 mut^{s} pAOX CES #5 was selected as basic production strain for CES secretion.

### Example 2: Generation of Engineered Strains Overexpressing Helper Genes

For the investigation of positive effects on recombinant protein secretion, the putative helper genes were overexpressed in the three basic production strains: CBS7435 mut^{S} pAOX scR (scFv) 4E3, CBS7435 mut^{S} pAOX Fab 9, and CBS7435 mut^{S} pAOX CES 5 (generation see Example 1).

### a) General Procedure of Amplification and Cloning of the Selected Genes.

The genes selected for overexpression were amplified by PCR (Q5® High-Fidelity DNA Polymerase, New England Biolabs) from start to stop codon or split into several fragments. The GoldenPiCS system (Prielhofer et al. 2017. BMC Systems Biol. doi: 10.1186/s12918-017-0492-3) requires the introduction of silent mutations in some coding sequences. This was performed by amplifying several fragments from one coding sequence. Alternatively, gBlocks or synthetic codon-optimized genes were obtained from commercial providers (including Integrated DNA Technology IDT, Geneart, and ATUM). Amplified coding sequences were either cloned into the pPUZZLE-based expression plasmids pPM2aK21 or pPM2eH21, or the GoldenPiCS system (consisting of the backbones B1l, BB2 and BB3aK/BB3eH/BB3rN). The gene fragments listed in Table 1 were introduced into B1l of the GoldenPiCS system by using the restriction enzyme BsaI. All promoters and terminators used to assemble expression cassettes in BB2 or BB3 backbones are described in Prielhofer et al. 2017 (BMC Systems Biol. doi: 10.1186/s12918-017-0492-3). pPM2aK21 and BB3aK allow integration into the 3′-AOX1 genomic region and contain the KanMX selection marker cassette for selection in E. coli and yeast. pPM2eH21 and BB3eH contain the 5′-ENO1 genome integration region and the HphMX selection marker cassette for selection on hygromycin. BB3rN contain the 5′-RG/1 genome integration region and the NatMX selection marker cassette for selection on nourseothricin. All plasmids contain an origin of replication for E. coli (pUC19). Genomic DNA from P. pastoris strain CBS7435 mut^{S} or gBlocks (Integrated DNA Technologies) served as PCR templates.

| Gene | Gene | |
|---|---|---|
| identifier | fragment | Cloned sequence |
| PP7435_Ch | Sbh1 spliced | GACAGGTCTCCCATGTCTACAGCAATTCCAGGAGGACAGAGAACGTTAGCT |
| r2-0220 | | AAAAGAAGAGCAGCAAACTTGGATAAGAAACAGGATGAACCAACCTCCGCC |
| | | AGATCTGCCGGTGCTGGAGGTTCTTCGTCTACCATGCTAAAGTTGTACACA |
| | | GACGAGGCCCAAGGTTTGAAAGTTGATCCTTTAATTGTTCTTGTTCTTGCTG |
| | | TTGGTTTCATTTTCAGTGTCATTGGTTTGCACGTTGTTGCTAAGCTGACAGG |
| | | AAAGTTGATCAACTAAGCTTGGAGACCGATC |
| | | (SEQ ID NO: 68) |
| PP7435_Ch | Sbh1 | 5′-GACAGGTCTCCCATGTCTACAGCAATTCCAGGAG-3′ |
| r2-0220 | spliced | (SEQ ID NO: 69) |
| | Kar2 | 5′-GATCGGTCTCCAAGCTTAGTTGATCAACTTTCCTGTCAG-3′ |
| | | (SEQ ID NO: 70) |
| PP7435_Ch | | GATCTAGGTCTCCCATGCTGTCGTTAAAACCATCTTGGCTGACTTTGGCGG |
| r2-1167 | | CATTAATGTATGCCATGCTATTGGTCGTAGTGCCATTTGCTAAACCTGTTAG |
| | | AGCTGACGATGTCGAATCTTATGGAACAGTGATTGGTATCGATTTGGGTAC |
| | | CACGTACTCTTGTGTCGGTGTGATGAAGTCGGGTCGTGTAGAAATTCTTGC |
| | | TAATGACCAAGGTAACAGAATCACTCCTTCCTACGTTAGTTTCACTGAAGAT |
| | | GAGAGACTGGTTGGTGATGCTGCTAAGAACTTAGCTGCTTCTAACCCAAAA |
| | | AACACCATCTTTGATATTAAGAGATTGATCGGTATGAAGTATGATGCCCCAG |
| | | AGGTCCAAAGAGACTTGAAGCGTCTGCCTTACACTGTCAAGAGCAAGAACG |
| | | GCCAACCTGTCGTTTCTGTCGAGTACAAGGGTGAGGAGAAGTCTTTCACTC |
| | | CTGAGGAGATTTCCGCCATGGTCTTGGGTAAGATGAAGTTGATCGCTGAGG |
| | | ACTACTTAGGAAAGAAAGTCACTCATGCTGTCGTTACCGTTCCAGCCTACTT |
| | | CAACGACGCTCAACGTCAAGCCACTAAGGATGCCGGTCTAATCGCCGGTTT |
| | | GACTGTTCTGAGAATTGTGAACGAGCCTACCGCCGCTGCCCTTGCTTACGG |
| | | TTTGGACAAGACTGGTGAGGAAAGACAGATCATCGTCTACGACTTGGGTGG |
| | | AGGAACCTTCGATGTTTCTCTGCTTTCTATTGAGGGTGGTGCTTTCGAGGTT |
| | | CTTGCTACCGCCGGTGACACCCACTTGGGTGGTGAGGACTTTGACTACAGA |
| | | GTTGTTCGCCACTTCGTTAAGATTTTCAAGAAGAAGCATAACATTGACATCA |
| | | GCAACAATGATAAGGCTTTAGGTAAGCTGAAGAGAGAGGTCGAAAAGGCCA |
| | | AGCGTACTTTGTCCTCCCAGATGACTACCAGAATTGAGATTGACTCTTTCGT |
| | | CGACGGTATCGACTTCTCTGAGCAACTGTCTAGAGCTAAGTTTGAGGAGAT |
| | | CAACATTGAATTATTCAAGAAAACACTGAAACCAGTTGAACAAGTCCTCAAA |
| | | GACGCTGGTGTCAAGAAATCTGAAATTGATGACATTGTCTTGGTTGGTGGTT |
| | | CTACCAGAATTCCAAAGGTTCAACAATTATTGGAGGATTACTTTGACGGAAA |
| | | GAAGGCTTCTAAGGGAATTAACCCAGATGAAGCTGTCGCATACGGTGCTGC |
| | | TGTTCAGGCTGGTGTTTTGTCTGGTGAGGAAGGTGTCGATGACATCGTCTT |
| | | GCTTGATGTGAACCCCCTAACTCTGGGTATCGAGACTACTGGTGGCGTTAT |
| | | GACTACCTTAATCAACAGAAACACTGCTATCCCAACTAAGAAATCTCAAATT |
| | | TTCTCCACTGCTGCTGACAACCAGCCAACTGTGTTGATTCAAGTTTATGAGG |
| | | GTGAGAGAGCCTTGGCTAAGGACAACAACTTGCTTGGTAAATTCGAGCTGA |
| | | CTGGTATTCCACCAGCTCCAAGAGGTACTCCTCAAGTTGAGGTTACTTTTGT |
| | | TTTAGACGCTAACGGAATTTTGAAGGTGTCTGCCACCGATAAGGGAACTGG |
| | | AAAATCCGAGTCCATCACCATCAACAATGATCGTGGTAGATTGTCCAAGGA |
| | | GGAGGTTGACCGTATGGTTGAAGAGGCCGAGAAGTACGCCGCTGAGGATG |
| | | CTGCACTAAGAGAAAAGATTGAGGCTAGAAACGCTCTGGAGAACTACGCTC |
| | | ATTCCCTTAGGAACCAAGTTACTGATGACTCTGAAACCGGGCTTGGTTCTAA |
| | | ATTGGACGAGGACGACAAAGAGACATTGACAGATGCCATCAAAGATACCCT |
| | | AGAGTTCTTGGAAGATAACTTCGACACCGCAACCAAGGAAGAATTAGACGA |
| | | ACAAAGAGAAAAGCTTTCCAAGATTGCTTACCCAATCACTTCTAAGCTATAC |
| | | GGTGCTCCAGAGGGTGGTACTCCACCTGGTGGTCAAGGTTTTGACGATGAT |
| | | GATGGAGACTTTGACTACGACTATGACTATGATCATGATGAGTTGTAAGCTT |
| | | GGAGACCAATGAC (SEQ ID NO: 71) |
| PP7435_Ch | Kar2 | 5′-GATCTAGGTCTCCCATGCTGTCGTTAAAACCATCT-3′ |
| r2-1167 | | (SEQ ID NO: 72) |
| | | 5′-GTCATTGGTCTCCAAGCTTACAACTCATCATGATCATAGTCATAG-3′ |
| | | (SEQ ID NO: NO: 73) |
| PP7435_Ch | Lhs1 | GATCTAGGTCTCCCATGAGAACACAAAAGATAGTAACAGTACTTTGTTTGCT |
| r1-0059 | | ACTAAATACTGTGCTTGGAGCTCTGTTGGGCATCGATTATGGTCAAGAGTTT |
| | | ACTAAGGCTGTCCTAGTGGCTCCTGGTGTCCCTTTTGAAGTTATCTTGACTC |
| | | CAGACTCCAAACGTAAAGATAATTCAATGATGGCCATCAAGGAAAATTCCAA |
| | | AGGTGAAATTGAGAGATATTATGGATCCTCAGCTAGTTCTGTTTGTATCAGA |
| | | AACCCTGAAACTTGCTTGAATCATCTGAAGTCATTGATAGGTGTTTCAATTG |
| | | ATGACGTTTCAACTATAGATTACAAGAAGTACCATTCAGGTGCTGAGATGGT |
| | | TCCATCCAAAAATAACAGGAACACGGTTGCCTTTAAGTTGGGCTCTTCTGTA |
| | | TATCCTGTAGAAGAGATACTTGCTATGAGTTTAGATGACATTAAATCTAGAG |
| | | CTGAAGATCATTTAAAACACGCGGTGCCAGGTTCCTATTCAGTTATCAGTGA |
| | | TGCTGTCATCACAGTACCCACTTTTTTTACCCAATCGCAAAGACTGGCCTTG |
| | | AAAGATGCTGCCGAAATTAGTGGCTTAAAAGTCGTTGGCTTGGTTGATGAC |
| | | GGTATATCTGTGGCCGTTAACTATGCCTCTTCAAGGCAGTTCAATGGAGAC |
| | | AAACAATATCATATGATCTATGACATGGGGGCTGGTTCTTTACAGGCGACTT |
| | | TGGTTTCTATATCTTCCAGTGATGATGGTGGAATTGTTATTGATGTAGAGGC |
| | | TATTGCCTATGACAAGTCGCTGGGAGGCCAGTTGTTCACACAATCTGTTTAT |
| | | GACATCCTTTTGCAGAAGTTCTTGTCTGAGCATCCTTCCTTTAGCGAGTCCG |
| | | ACTTCAACAAGAATAGTAAATCTATGTCAAAACTTTGGCAAGCGGCTGAAAA |
| | | GGCAAAGACAATTTTGAGTGCAAACACTGACACAAGAGTTTCCGTTGAATC |
| | | CTTATACAATGACATTGACTTTAGAGCCACAATAGCAAGAGACGAATTCGAA |
| | | GATTACAATGCAGAGCATGTTCATAGGATCACTGCTCCTATCATCGAGGCC |
| | | TTAAGTCATCCATTGAATGGGAATCTGACGTCACCTTTTCCACTGACCAGTT |
| | | TAAGTTCAGTAATTCTCACAGGGGGGTCAACAAGAGTGCCGATGGTGAAAA |
| | | AGCACCTAGAATCTTTGCTAGGATCTGAATTGATTGCAAAGAATGTTAACGC |
| | | TGATGAGTCAGCCGTTTTTGGTTCTACTCTCCGTGGTGTAACTTTATCGCAA |
| | | ATGTTCAAAGCGAAACAGATGACCGTAAATGAAAGAAGTGTATATGACTATT |
| | | GCCTAAAAGTTGGTTCTTCAGAGATAAACGTGTTCCCAGTTGGCACCCCTC |
| | | TTGCTACTAAGAAAGTGGTCGAGCTGGAAAATGTAGACAGTGAGAACCAGC |
| | | TCACGATTGGGCTCTACGAGAACGGACAATTGTTTGCCAGTCATGAGGTTA |
| | | CAGACCTCAAGAAGAGTATCAAATCTCTAACTCAAGAAGGTAAAGAGTGTTC |
| | | TAATATTAATTACGAGGCTACAGTCGAGTTATCTGAGAGCAGATTGCTTTCT |
| | | TTAACTCGTCTGCAGGCCAAATGTGCTGACGAGGCTGAATATTTACCTCCT |
| | | GTGGACACAGAGTCTGAGGATACTAAATCTGAAAACTCAACTACTAGTGAG |
| | | ACTATTGAAAAACCAAACAAGAAGCTATTCTATCCTGTGACTATACCTACTC |
| | | AACTGAAATCCGTTCACGTGAAACCAATGGGGTCCTCTACCAAGGTATCTT |
| | | CATCTTTGAAAATCAAGGAGTTGAACAAGAAGGATGCTGTAAAGAGATCGAT |
| | | CGAAGAATTGAAGAATCAGCTGGAATCGAAATTATACCGCGTGCGCTCGTA |
| | | TTTAGAGGATGAGGAAGTGGTTGAAAAAGGGCCAGCATCACAAGTTGAGGC |
| | | TTTGTCAACACTGGTTGCTGAGAATCTTGAGTGGTTGGACTATGATAGCGA |
| | | CGATGCATCAGCAAAAGATATCAGGGAAAAACTAAATTCTGTGTCAGATAGT |
| | | GTTGCCTTCATCAAGAGCTACATTGATCTGAACGATGTCACTTTTGATAATA |
| | | ATCTTTTCACTACGATTTACAACACTACTTTAAACTCCATGCAAAATGTTCAA |
| | | GAACTAATGTTAAACATGAGTGAGGATGCTCTGAGTTTAATGCAGCAGTATG |
| | | AGAAGGAAGGTTTAGACTTCGCCAAAGAAAGTCAAAAGATCAAAATAAAATC |
| | | TCCTCCTTTATCAGACAAAGAGCTTGATAATCTCTTTAACACTGTTACCGAAA |
| | | AGTTAGAGCATGTCAGAATGTTGACTGAAAAGGACACTATAAGTGATTTGCC |
| | | TAGAGAGGAGCTTTTTAAGCTGTATCAAGAATTGCAGAACTACTCTTCCCGA |
| | | TTTGAAGCAATCATGGCCAGTTTGGAAGATGTACACTCTCAAAGAATCAACC |
| | | GTTTGACAGACAAGTTACGCAAACATATTGAAAGGGTGAGCAATGAAGCAT |
| | | TGAAGGCAGCTCTCAAGGAAGCTAAACGTCAACAAGAGGAGGAAAAAAGC |
| | | CACGAGCAGAATGAGGGAGAAGAGCAAAGTTCTGCTTCCACTTCTCACACT |
| | | AATGAAGATATAGAGGAACCATCAGAATCGCCTAAGGTTCAAACATCCCAT |
| | | GATGAGTTGTAAGCTTGGAGACCAATGAC |
| | | (SEQ ID NO: 77) |
| PP7435_Ch | Lhs1 | 5′-GATCTAGGTCTCCCATGAGAACACAAAAGATAGTAACAGTAC-3′ |
| r1-0059 | | (SEQ ID NO: 78) |
| | | 5′-GTCATTGGTCTCCAAGCTTACAACTCATCATGGGATGTTT-3′ |
| | | (SEQ ID NO: 79) |
| PP7435_Ch | Sil1 | GATCTAGGTCTCCCATGAAAGTGACATTATCTGTGTTAGCTATTGCCTCCCA |
| r1-0550 | | ATTGGTTAGAATCGTTTGTTCGGAAGGAGAAAATATCTGCATAGGTGACCA |
| | | GTGCTATCCGAAGAATTTTGAACCTGACAAGGAGTGGAAACCTGTTCAGGA |
| | | AGGCCAGATTATCCCTCCAGGATCACACGTAAGAATGGACTTTAATACACA |
| | | CCAGAGAGAGGCAAAACTGGTGGAAGAGAATGAGGATATAGACCCCTCAT |
| | | CATTGGGAGTGGCTGTAGTGGATTCCACCGGTTCGTTTGCTGATGATCAAT |
| | | CTTTGGAAAAGATTGAGGGACTTTCCATGGAACAACTAGATGAGAAGTTAG |
| | | AAGAACTGATTGAGCTTTCCCATGACTACGAGTACGGATCAGACATAATCTT |
| | | GAGTGATCAGTATATTTTTGGAGTAGCCGGGCTAGTTCCTACTAAGACAAA |
| | | GTTTACTTCTGAGTTGAAGGAAAAGGCCTTGAGAATTGTCGGATCATGCTTG |
| | | AGAAACAATGCCGATGCGGTAGAGAAACTACTGGGAACTGTTCCAAATACT |
| | | ATAACCATACAATTCATGTCAAACCTAGTGGGTAAAGTAAATTCCACTGGAG |
| | | AGAATGTTGACTCTGTTGAACAGAAACGAATCCTTTCAATTATTGGAGCTGT |
| | | TATTCCTTTCAAAATTGGAAAGGTATTGTTTGAAGCTTGTTCGGGAACGCAG |
| | | AAGCTATTACTATCCTTGGATAAACTGGAAAGTTCAGTTCAACTGAGAGGAT |
| | | ACCAAATGTTGGACGACTTCATTCATCACCCTGAAGAGGAACTTCTCTCTTC |
| | | ATTGACAGCAAAGGAACGATTAGTAAAGCATATTGAGTTGATTCAATCATTT |
| | | TTTGCATCAGGAAAGCATTCTCTTGATATAGCAATAAATCGTGAGTTATTCA |
| | | CTAGGCTGATTGCCTTACGAACCAATTTAGAATCTGCCAATCCAAATCTATG |
| | | TAAACCATCAACTGACTTTTTGAACTGGCTGATCGACGAAATTGAAGCTACG |
| | | AAAGATACCGATCCACACTTTTCAAAAGAGCTTAAACATTTACGTTTTGAACT |
| | | TTTTGGGAACCCATTGGCATCTAGGAAAGGTTTCTCCGATGAGTTATAAGCT |
| | | TGGAGACCAATGAC |
| | | (SEQ ID NO: 80) |
| PP7435_Ch | Sil1 | 5′-GATCTAGGTCTCCCATGAAAGTGACATTATCTGTGTTAGC-3′ |
| r1-0550 | | (SEQ ID NO: 81) |
| | | 5′-GTCATTGGTCTCCAAGCTTATAACTCATCGGAGAAACCTTTC-3′ |
| | | (SEQ ID NO: 82) |
| PP7435_Ch | Erj5 | GATCTAGGTCTCCCATGAAACTACACCTTGTGATTCTCTGTTTGATCACTGC |
| r1-0136 | | TGTCTACTGTTTCAGTGCTGTTGACAGAGAAATCTTTCAGCTCAACCATGAA |
| | | TTACGCCAGGAATACGGAGATAATTTTAATTTCTATGAATGGTTGAAGCTTC |
| | | CAAAAGGTCCCTCGTCCACGTTTGAAGATATCGACAACGCGTACAAGAAAC |
| | | TATCCCGTAAGTTACACCCCGATAAGATAAGACAGAAGAAACTATCCCAGG |
| | | AACAATTTGAGCAATTGAAGAAAAAGGCTACCGAAAGATACCAACAATTGAG |
| | | TGCTGTGGGATCCATCTTAAGATCCGAGAGCAAAGAGCGTTACGATTATTTT |
| | | GTCAAACATGGATTCCCAGTCTATAAAGGTAACGATTACACCTATGCCAAGT |
| | | TTAGACCATCCGTTTTGCTCACAATTTTCATCCTTTTTGCGTTAGCTACGTTA |
| | | ACCCACTTTGTCTTTATCAGATTGTCGGCCGTGCAATCTAGAAAAAGACTGA |
| | | GTTCGTTGATAGAGGAGAACAAACAGCTGGCTTGGCCACAAGGTGTTCAAG |
| | | ATGTCACTCAAGTGAAGGACGTCAAAGTCTATAACGAACATCTACGTAAATG |
| | | GTTTTTGGTATGTTTCGACGGATCCGTTCATTATGTGGAGAACGATAAAACC |
| | | TTCCATGTTGATCCGGAAGAAGTTGAACTCCCATCTTGGCAGGACACTCTT |
| | | CCAGGTAAATTAATAGTCAAGCTGATACCCCAGCTTGCTAGAAAGCCACGA |
| | | TCTCCAAAGGAGATCAAGAAGGAAAATTTAGATGATAAAACCAGAAAGACAA |
| | | AAAAACCTACAGGGGATTCCAAAACTTTACCTAACGGTAAAACCATTTATAA |
| | | AGCTACCAAATCCGGTGGACGTAGAAGGAAATAAGCTTGGAGACCAATGAC |
| | | (SEQ ID NO: 83) |
| PP7435_Ch | Erj5 | 5′-GATCTAGGTCTCCCATGAAACTACACCTTGTGATTCTC-3′ |
| r1-0136 | | (SEQ ID NO: 84) |
| | | 5′-GTCATTGGTCTCCAAGCTTATTTCCTTCTACGTCCACC-3′ |
| | | (SEQ ID NO: 85) |
| PP7435_Ch | Hac1 (i) | GATCTAGGTCTCACATGCCCGTAGATTCTTCTCATAAGACAGCTAGCCCAC |
| r1-0700 | | TTCCACCTCGTAAAAGAGCAAAGACGGAAGAAGAAAAGGAGCAGCGTCGA |
| | | GTGGAACGTATCCTACGTAATAGGAGAGCGGCCCATGCTTCCAGAGAGAA |
| | | GAAACGTAGACACGTTGAATTTCTGGAAAACCACGTCGTCGACCTGGAATC |
| | | TGCACTTCAAGAATCAGCCAAAGCCACTAACAAGTTGAAAGAAATACAAGAT |
| | | ATCATTGTTTCAAGGTTGGAAGCCTTAGGTGGTACCGTCTCAGATTTGGATT |
| | | TAACAGTTCCGGAAGTCGATTTTCCCAAATCTTCTGATTTGGAACCCATGTC |
| | | TGATCTCTCAACTTCTTCGAAATCGGAGAAAGCATCTACATCCACTCGCAGA |
| | | TCTTTGACTGAGGATCTGGACGAAGATGACGTCGCTGAATATGACGACGAA |
| | | GAAGAGGACGAAGAGTTACCCAGGAAAATGAAAGTCTTAAACGACAAAAAC |
| | | AAGAGCACATCTATCAAGCAGGAGAAGTTGAATGAACTTCCATCTCCTTTGT |
| | | CATCCGATTTTTCAGACGTAGATGAAGAAAAGTCAACTCTCACACATTTAAA |
| | | GTTGCAACAGCAACAACAACAACCAGTAGACAATTATGTTTCTACTCCTTTG |
| | | AGTCTGCCGGAGGATTCAGTTGATTTTATTAACCCAGGTAACTTAAAAATAG |
| | | AGTCCGATGAGAACTTCTTGTTGAGTTCAAATACTTTACAAATAAAACACGA |
| | | AAATGACACCGACTACATTACTACAGCTCCATCAGGTTCCATCAATGATTTT |
| | | TTTAATTCTTATGACATTAGCGAGTCGAATCGGTTGCATCATCCAGCAGCAC |
| | | CATTTACCGCTAATGCATTTGATTTAAATGACTTTGTATTCTTCCAGGAATAG |
| | | TAGGCTTCGAGACCAATGAC |
| | | (SEQ ID NO: 74) |
| PP7435_Ch | Hac1 (i) | 5′-GATCTAGGTCTCACATGCCCGTAGATTCTTCTC-3′ |
| r1-0700 | | (SEQ ID NO: 75) |
| | | 5′-GTCATTGGTCTCGAAGCCTACTATTCCTGGAAGAATACAAAG-3′ |
| | | (SEQ ID NO: 76) |
| | Hac1 (i) | ATGCCTGTCGATTCTTCTCACAAGACCGCTAGTCCACTACCACCTAGAAAG |
| | optimized | AGAGCAAAGACTGAAGAGGAAAAGGAACAGAGAAGAGTCGAACGTATTCT |
| | | CAGGAATAGACGTGCTGCCCACGCTTCAAGAGAGAAAAAGCGTAGACACG |
| | | TCGAGTTCCTAGAGAATCATGTTGTGGACCTGGAATCGGCCCTCCAAGAGA |
| | | GCGCTAAGGCAACCAACAAGCTTAAAGAGATTCAAGACATTATTGTCAGCA |
| | | GACTAGAAGCTCTAGGAGGTACTGTCTCCGATTTGGACCTGACTGTTCCAG |
| | | AAGTAGACTTTCCCAAGTCCTCTGATTTGGAGCCAATGTCAGACCTTTCTAC |
| | | TTCTTCCAAGTCGGAAAAGGCTTCCACCTCTACTAGAAGATCCCTTACAGA |
| | | GGACCTGGACGAGGACGACGTTGCTGAATACGATGACGAGGAGGAAGATG |
| | | AGGAACTGCCTAGAAAAATGAAAGTTCTCAACGACAAAAACAAATCTACCTC |
| | | TATCAAGCAAGAGAAGCTTAACGAGCTGCCATCTCCACTAAGTTCTGATTTT |
| | | TCCGACGTTGACGAAGAGAAGTCAACCCTAACCCACCTCAAGTTACAACAG |
| | | CAACAACAACAGCCTGTTGACAACTATGTTTCTACGCCTCTGTCTTTGCCAG |
| | | AAGATTCTGTGGATTTCATTAACCCAGGTAACCTCAAGATCGAATCCGACG |
| | | AAAACTTCCTGCTTTCCTCTAACACACTTCAAATCAAGCACGAGAACGATAC |
| | | TGACTACATTACTACTGCTCCTTCTGGTTCTATCAACGACTTTTTCAATTCAT |
| | | ACGACATCTCCGAATCTAACAGACTACATCACCCTGCCGCTCCATTTACCG |
| | | CAAACGCCTTCGATCTCAATGACTTCGTTTTCTTCCAGGAGTAATAG |
| | | (SEQ ID NO: 86) |
| | Hac1(i) | ATGCCAGTTGATAGTTCGCACAAGACTGCTTCTCCACTGCCACCTAGAAAG |
| | optimized | AGAGCTAAGACTGAGGAGGAAAAGGAGCAACGTAGAGTCGAGAGAATCCT |
| | | GAGAAACCGTAGAGCCGCTCACGCCTCTAGAGAGAAAAAGAGAAGGCATG |
| | | TTGAATTTCTTGAAAACCACGTCGTCGATCTCGAATCTGCCCTTCAAGAGTC |
| | | AGCTAAAGCTACCAACAAGCTAAAGGAAATTCAAGACATTATCGTATCTAGA |
| | | CTGGAGGCACTTGGTGGTACTGTTTCTGACCTGGATCTTACAGTTCCAGAA |
| | | GTTGACTTCCCAAAATCCAGTGATCTAGAACCTATGTCTGATCTATCTACCT |
| | | CAAGCAAGTCTGAGAAGGCAAGCACGTCAACCAGACGTTCCCTAACTGAG |
| | | GACCTGGACGAAGATGATGTCGCTGAATACGATGACGAGGAGGAGGATGA |
| | | GGAACTGCCTAGAAAAATGAAGGTTCTTAACGACAAAAACAAGTCTACCTC |
| | | TATCAAACAGGAAAAGCTCAACGAACTCCCATCCCCTCTCTCTTCCGACTT |
| | | CTCCGACGTGGACGAGGAAAAGTCTACTTTGACCCACCTGAAGTTGCAACA |
| | | ACAACAGCAACAACCTGTTGACAACTATGTCTCCACTCCTCTCTCACTCCCA |
| | | GAGGACTCGGTTGACTTCATCAACCCCGGTAACCTTAAGATTGAATCTGAC |
| | | GAGAACTTCCTTCTATCCTCTAATACCTTACAGATTAAGCATGAAAATGATA |
| | | CTGACTACATTACTACCGCTCCATCCGGATCTATCAATGACTTCTTCAATTC |
| | | TTACGACATTTCTGAGTCCAACAGATTGCACCACCCAGCTGCACCTTTTACA |
| | | GCCAACGCTTTTGACCTAAACGACTTCGTGTTTTTCCAGGAGTAATAG |
| | | (SEQ ID NO: 87) |
| | Hac1(i) | ATGCCTGTAGATTCCTCCCACAAGACTGCTTCGCCACTTCCTCCTCGTAAG |
| | optimized | AGAGCTAAGACCGAAGAGGAAAAGGAACAGAGAAGAGTTGAGAGAATCCT |
| | | GAGAAACAGAAGGGCTGCTCACGCCAGTAGAGAGAAAAAGCGTCGTCACG |
| | | TCGAGTTCCTAGAAAATCATGTTGTTGATTTGGAATCAGCCCTGCAAGAGT |
| | | CTGCCAAAGCAACCAACAAGCTGAAGGAGATCCAGGACATTATTGTCTCTA |
| | | GACTTGAAGCTCTAGGTGGTACTGTCTCTGACCTAGACCTCACCGTGCCTG |
| | | AGGTTGACTTTCCAAAGTCGTCTGATCTCGAACCAATGTCTGACCTATCAAC |
| | | TTCATCTAAGTCTGAGAAGGCAAGCACCTCTACCAGAAGATCCCTTACAGA |
| | | GGACCTCGATGAAGATGACGTTGCTGAATATGATGACGAAGAGGAGGACG |
| | | AGGAATTACCTAGAAAGATGAAGGTCTTGAACGACAAAAACAAATCCACTT |
| | | CTATTAAGCAGGAAAAGCTAAACGAGCTGCCATCTCCACTCTCCTCTGATTT |
| | | CAGCGACGTGGACGAGGAGAAATCTACACTCACTCACCTTAAGCTACAACA |
| | | ACAGCAACAACAACCCGTTGACAACTACGTTTCCACCCCACTTTCTCTCCC |
| | | AGAGGACAGTGTTGATTTCATTAACCCTGGAAACCTGAAAATCGAGTCAGA |
| | | CGAAAACTTCCTACTGTCCTCCAACACGCTTCAAATCAAGCACGAAAATGA |
| | | CACTGATTACATTACTACTGCTCCATCTGGTTCTATTAACGACTTTTTCAATT |
| | | CCTACGATATTTCTGAATCTAACAGATTGCACCATCCTGCTGCCCCATTCAC |
| | | CGCAAACGCTTTTGACCTCAATGACTTCGTCTTTTTCCAAGAGTAATAG |
| | | (SEQ ID NO: 88) |

b) Creating the sbh1 Overexpression Strains.

The coding sequence of sbh1 without the intron sequence (from nucleotide 3 to 67 bp) was introduced into B1l of the GoldenPiCS system.

This coding sequence (see Table 1) was combined with the glyceraldehyde-3-phosphate dehydrogenase (GAP) promoter and the native RPS3 transcription terminator into the integration plasmid BB3rN (185_BB3rN). SBH1 was also combined with pRPL2A promoter and RPS3 terminator (186_BB3rN) or the pMDH2 promoter and RPS3 terminator (192_BB3rN). All integration plasmids were linearized with the restriction enzyme AscI prior to their application for transforming the basic production strains. Titer and yield (titer per wet cell weight) of the clones overexpressing sbh1 was determined in small scale screenings and compared to their parental basic production strains (Example 3).

c) Creating the Strains Overexpressing sbh1+kar2 or sbh1+lhs1 or sbh1+sil1.

The coding sequences of kar2 (7 silent mutations required), Ihs1 (1 silent mutation required), sil1 (no mutations) and erj5 (1 silent mutation required) were introduced into B1E of the GoldenPiCS system.

An overexpression cassette only containing kar2 was assembled in the integration plasmid BB3eH (219_BB3eH). This plasmid derives from combining the E1E plasmids with the kar2 coding sequence and the GAP promoter as well as the RPS3 terminator.

An overexpression cassette only containing Ihs1 was assembled in the integration plasmid BB3eH (417_BB3eH). This plasmid derives from combining the E1E plasmids with the Ihs1 coding sequence and the POR1 promoter and the IDP1 transcription terminator.

An overexpression cassette only containing sil1 was assembled in the integration plasmid BB3eH (418_BB3eH). This plasmid derives from combining the E1E plasmids with the sil1 coding sequence and the POR1 promoter and the IDP1 transcription terminator.

After transformation with the respective plasmid of Example 2b, the best clones overexpressing sbh1 in terms of product yield determined in small scale screenings (Example 3) were chosen and further transformed with the Smal linearized kar2 expression plasmid 219_EE3eH or the xxx linearized Ihs1 expression plasmid (417_EE3eH) or the xxx linearized sil1 expression plasmid (418_BB3eH). This finally yielded clones with two different overexpression cassettes introduced by two sequential transformations with two different integration plasmids.

Alternatively, both helper factors could be combined on one expression plasmid as described below.

d) Creating the Strains Overexpressing sbh1+hac1(i).

The induced (i) version of the hac1(i) coding sequence was created by removing the alternative intron from nucleotide no. 857 to 1178 according to Guerfal et al. 2010 (Microb Cell Fact. doi: 10.1186/1475-2859-9-49). The coding sequence was introduced into B1l. It was further combined with the promoter of FDH1 and the terminator of RPL2A in a 234_BB3eH plasmid. Other BB3 constructs contained hac1(i) under control of the MDH3 promoter and the RPL2A terminator, or the ADH2 promoter and the RPL2A terminator (data not shown).

After transformation with the respective plasmid of Example 2b, the best clones overexpressing sbh1 in terms of product yield determined in small scale screenings (Example 3) were chosen and further transformed with the Smal linearized hac1(i) expression plasmid. This finally yielded clones with two different overexpression cassettes introduced by two sequential transformations with two different integration plasmids. Alternatively, both helper factors could be combined on one expression plasmid as described below.

e) Creating the sbh1+kar2+Ihs1, sbh1+kar2+sil1, and sbh1+kar2+(Ihs1 or sil1)+erj5 Overexpression Strains.

The overexpression of kar2 in combination with Ihs1 was assembled in the integration plasmid 174_BB3eH, which derives from two BB2s; one containing kar2 with the GAP promoter and the RPS3 transcription terminator and the other BB2 containing Ihs1 with the POR1 promoter and the IDP1 transcription terminator. The overexpression of kar2 in combination with sil1 was assembled in the integration plasmid 078_BB3eH, which derives from two BB2s; one containing kar2 with the GAP promoter and the RPS3 transcription terminator and the other BB2 containing sil1 with the POR1 promoter and the IDP1 transcription terminator. The overexpression of kar2 in combination with Ihs1 and erj5 was assembled in the integration plasmid 052_BB3eH, which derives from three BB2s; the first containing kar2 with the GAP promoter and the S. cerevisiae CYC1 transcription terminator, the second BB2 containing Ihs1 with the POR1 promoter and the IDP1 transcription terminator and the third BB2 containing erj5 with the MDH3 promoter and the TDH1 transcription terminator.

After transformation with the respective plasmid of Example 2b, the best clones overexpressing SBH1 in terms of product yield determined in small scale screenings (Example 3) were chosen and further transformed with the respective Smal linearized BB3eH integration plasmid mentioned above. This finally yielded clones with two different overexpression cassettes introduced by two sequential transformations with two different integration plasmids. Alternatively, all helper factors could be combined on a single plasmid containing multiple sequential expression cassettes.

### Example 3: Screening for Increased scFv, Fab or CES Secretion

In small-scale screenings, up to 20 transformants of each overexpression construct (overexpressing either Sbh1 alone or in combination with Kar2 or Hac1(i) plus Lsh1, Sil1, or Erj5) were tested after transformation. These transformants were evaluated by comparing their scFv, Fab or CES titer in the supernatant (mg POI/L culture supernatant or whole cell broth), their biomass concentration (g WCW/L culture) and their scFv, Fab or CES yield (mg POI/g biomass measured as WCW) to those of the respective parental basic production strain. For each overexpression combination an average fold-change of titer, yield and wet cell weight was determined to assess the secretion improvement. The average fold-change of titer, yield and wet cell weight was calculated by dividing the arithmetic mean of titer, yield and wet cell weight of all transformants by the arithmetic mean of titer, yield and wet cell weight of the four biological replicates of the basic production strains cultivated on the same deep well plate.

a) Small Scale Screening Cultivations of scFv, Fab or CES Production Strains.

2 mL YP-medium (10 g/L yeast extract, 20 g/L peptone) containing 10 g/L glucose and 50 μg/mL Zeocin (basic production strains) or 50 μg/mL Zeocin and 500 μg/mL G418 and/or 200 μg/mL Hygromycin and/or 100 μg/mL Nourseothricin (depending on the integration plasmids of the engineered strains) were inoculated with a single colony of a P. pastoris clone and grown overnight at 25° C. These cultures were transferred to 2 mL of synthetic screening medium M2 or ASMv6 (media compositions are given below) supplemented with a glucose feed tablet (Kuhner, Switzerland; CAT #SMFB63319) or 0.35% of enzyme (m2p media development kit) and incubated for 1 to 25 h at 25° C. at 280 rpm in 24 deep well plates. Aliquots of these cultures (corresponding to a final OD₆₀₀ of 4 or 8) were transferred into 2 mL of synthetic screening medium M2 or ASMv6 (in the case of ASMv6 with the m2p media development kit in fresh 24 deep well plates. 0.5 vol % of pure methanol were added initially and 1 vol % of pure methanol were repeatedly added after 19 hours, 27 hours, and 43 hours. After 48 hours, the cells were harvested by centrifugation at 2,500×g for 10 min at room temperature and prepared for analysis. Biomass was determined by measuring the cell weight of 1 mL cell suspension, while determination of the recombinant secreted protein in the supernatant is described in the following Examples 3b-3c.

Synthetic screening medium M2 contained per liter: 22.0 g Citric acid monohydrate 3.15 g (NH₄)₂HPO₄, 0.49 g MgSO₄*7H₂O, 0.80 g KCl, 0.0268 g CaCl₂*2H₂O, 1.47 mL PTM1 trace metals, 0.4 mg Biotin; pH was set to 5 with KOH (solid).

Synthetic screening medium ASMv6 contained per liter: 22.0 g Citric acid monohydrate, 6.30 g (NH₄)₂HPO₄, 0.8 g (NH₄)₂SO₄, 0.49 g MgSO₄*7H₂O, 2.64 g KCl, 0.0535 g CaCl₂*2H₂O, 1.47 mL PTM1 trace metals, 0.4 mg Biotin; pH was set to 6.5 with KOH (solid)

### b) SDS-PAGE & Western Blot Analysis.

For protein gel analysis the NuPAGE® Novex® Bis-Tris system was used, using 12% Bis-Tris gels with MOPS running buffer or 4-12% Bis-Tris gels with MES running buffer (all from Invitrogen). After electrophoresis, the proteins were either visualized by colloidal Coomassie staining or transferred to a nitrocellulose membrane for Western blot analysis. Therefore, the proteins were electroblotted onto a nitrocellulose membrane using the Biorad Trans-Blot® Turbo™ Transfer System with ready-to-use membranes and filter papers and the program Turbo for minigels (7 min). After blocking, the Western Blots were probed with the following antibodies: The His-tagged scFv and CES were detected with the following antibody: Anti-polyHistidin-Peroxidase antibody (A7058, Sigma), diluted 1:2,000; For Fab light chain: anti-human kappa light chains (bound and free)—alkaline phosphatase (AP) conjugated antibody, Sigma A3813 (1:5,000); For Fab heavy chain: Mouse Anti-Human IgG antibody (Ab7497, Abcam) diluted 1:1.000 and Anti-Mouse IgG (Fc specific)—Alkaline Phosphatase antibody produced in goat (A1418, Sigma) as secondary antibody diluted 1:5.000.

Detection was performed with the colorimetric AP detection kit (BioRad) based on the NBT/BCIP system for AP-conjugates, and the chemiluminescent Super Signal West Chemiluminescent Substrate (Thermo Scientific) for HRP-conjugates.

c) Quantification by Microfluidic Capillary Electrophoresis (mCE).

The ‘LabChip GX/GXII System’ (PerkinElmer) was used for quantitative analysis of secreted protein titer in culture supernatants. The consumables ‘Protein Express Lab Chip’ (760499, PerkinElmer) and ‘Protein Express Reagent Kit’ (CLS960008, PerkinElmer) were used. Briefly, several μL of all culture supernatants are fluorescently labeled and analyzed according to protein size, using an electrophoretic system based on microfluidics. Internal standards enable approximate allocations to size in kDa and approximate concentrations of detected signals.

### d) Quantification of Fab by ELISA.

Fab samples were also analyzed by enzyme-linked immunosorbent assay (ELISA). Fab-ELISA was done using anti-human IgG antibody (ab7497, Abcam) and a goat anti-Human Kappa Light Chain (Bound and Free)—alkaline phosphatase conjugated antibody (Sigma A3813) for the quantification of intact Fab. Human Fab/Kappa, IgG fragment (Bethyl P80-115) was used as standard and detection was done with pNPP (Sigma S0942).

Coating-, Dilution- and Washing buffer were based on PBS (2 mM KH2PO4, 10 mM Na2HPO4.2H2O, 2.7 mM g KCl, 8 mM NaCl, pH 7.4) and completed with BSA (1% (w/v)) and/or Tween20 (0.1% (v/v)) accordingly.

### Example 4: Fed Batch Cultivations

Clones of the engineered strains (Example 2) were selected after small scale screening cultivations (Example 3). The selected clones were further evaluated in larger cultivation volumes by fed batch bioreactor cultivations. Secretion improvements in small scale screenings, which were also present in fed batch bioreactor cultivations, were verified.

### a) Procedure of Fed Batch Bioreactor Cultivations.

Respective strains were inoculated into wide-necked, baffled, covered 300 mL shake flasks filled with 50 mL of YPhyG and shaken at 110 rpm at 28° C. over-night (pre-culture 1). Pre-culture 2 (100 mL YPhyG in a 1000 mL wide-necked, baffled, covered shake flask) was inoculated from pre-culture 1 in a way that the OD₆₀₀ (optical density measured at 600 nm) reached approximately 20 (measured against YPhyG media) in late afternoon (doubling time: approximately 2 hours). Incubation of pre-culture 2 was performed at 110 rpm at 28° C., as well.

The fed batches were carried out in 0.8 L working volume bioreactors (Minifors, Infors, Switzerland). All bioreactors (filled with 400 mL BSM-media with a pH of approximately 5.5) were individually inoculated from pre-culture 2 to an OD₆₀₀ of 2.0. Generally, P. pastoris was grown on glycerol to produce biomass and the culture was subsequently subjected to glycerol feeding followed by methanol feeding.

In the initial batch phase, the temperature was set to 28° C. Over the period of the last hour before initiating the production phase it was decreased to 24° C. and kept at this level throughout the remaining process, while the pH dropped to 5.0 and was kept at this level. Oxygen saturation was set to 30% throughout the whole process (cascade control: stirrer, flow, oxygen supplementation). Stirring was applied between 700 and 1200 rpm and a flow range (air) of 1.0-2.0 L/min was chosen. Control of pH at 5.0 was achieved using 25% ammonium. Foaming was controlled by addition of antifoam agent Glanapon 2000 on demand.

During the batch phase, biomass was generated (μ~0.30/h) up to a wet cell weight (WCW) of approximately 110-120 g/L. The classical batch phase (biomass generation) would last about 14 hours. Glycerol was fed with a rate defined by the equation 2.6+0.3*t (g/h), so a total of 30 g glycerol (60%) was supplemented within 8 hours. The first sampling point was selected to be 20 hours (0 h induction time).

In the following 18 hours (from process time 20 to 38 hours), a mixed feed of glycerol/methanol was applied: glycerol feed rate defined by the equation: 2.5+0.13*t (g/h), supplying 66 g glycerol (60%) and methanol feed rate defined by the equation: 0.72+0.05*t (g/h), adding 21 g of methanol.

During the next 72-74 hours (from process time 38 to 110-112 hours) methanol was fed with a feed rate defined by the equation 2.2+0.016*t (g/L)).

YPhyG preculture medium (per liter) contained: 20 g Phytone-Peptone, 10 g Bacto-Yeast Extract, 20 g glycerol.

Batch medium: Modified Basal salt medium (BSM) (per liter) contained: 13.5 mL H₃PO₄ (85%), 0.5 g CaCl 2H₂O, 7.5 g MgSO₄, 7H₂O, 9 g K₂SO₄, 2 g KOH, 40 g glycerol, 0.25 g NaCl, 4.35 mL PTM1, 0.1 mL Glanapon 2000 (antifoam).

PTM1 Trace Elements (per liter) contains: 0.2 g Biotin, 6.0 g CuSO₄·5H₂O, 0.09 g KI, 3.00 g MnSO₄·H₂O, 0.2 g Na₂MoO₄·2H₂0, 0.02 g H₃BO₃, 0.5 g CoC₂, 42.2 g ZnSO₄·7H₂O, 65.0 g FeSO₄·7H₂O, and 5.0 mL H₂SO₄ (95%-98%).

Feed-solution glycerol (per kg) contained: 600 g glycerol, 12 mL PTM1. Feed-solution methanol contained: pure methanol.

### b) Sample Analysis of Fed Batch Bioreactor Cultivations.

Samples were taken at various time points with the following procedure: the first 3 mL of sampled cultivation broth (with a syringe) were discarded. 1 mL of the freshly taken sample (3-5 mL) was transferred into a 1.5 mL centrifugation tube and spun for 5 minutes at 13,200 rpm (16,100 g). Supernatants were diligently transferred into a separate vial and stored at 4° C. or frozen until analysis.

1 mL of cultivation broth was centrifuged in a tared Eppendorf vial at 13,200 rpm (16,100 g) for 5 minutes and the resulting supernatant was accurately removed. The vial was weighed (accuracy 0.1 mg), and the tare of the empty vial was subtracted to obtain wet cell weights.

Supernatants of the individual sampling points of each bioreactor cultivation were analyzed using mCE (microfluidic capillary electrophoresis, GXII, Perkin-Elmer) against BSA or purified standard material (scR-GG-6×HIS or CES-GG-6HIS). Fab was additionally quantified by ELISA.

### Example 5: Improvement of Recombinant Protein Production and Secretion by Overexpressions of a Translocation Pore Subunit and Helper Gene(s) in Small Scale Screenings

The secretion improvement is measured by titer and yield fold-change values that refer to the respective non-engineered basic production strains (=parental strain) (Example 1). The fold-change values of small scale screenings are the arithmetic mean of up to 20 clones/transformants compared to four replicates of the parental strain cultivated at the same deep well plate (see Example 3).

a) Improvement of Fab Protein Secretion Yields by Overexpression of a Translocation Pore Subunit Alone or in Combination with Helper Gene(s)—Results from Small Scale Screenings

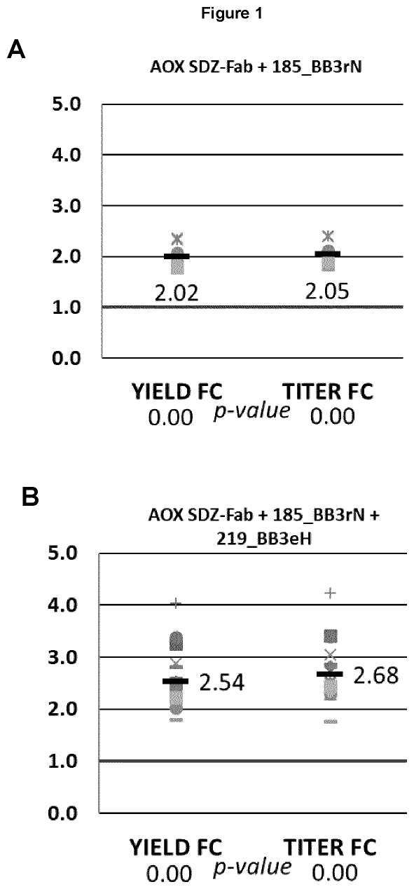

FIGS. 1-3 show the effect of overexpressing sbh1 alone or in combination with helper gene(s) in the Fab producing P. pastoris strain pAOX1-Fab #9 in small scale screenings (Example 3).

Secretion of Fab is approximately 1.5- to 2-fold increased by overexpression of the translocation subunit Sbh1 alone (FIG. 1A). Overexpression of Kar2 alone improved Fab production by 1.5-fold on average. When these two genes were co-overexpressed (sbh1+kar2), Fab titers and yields were increased even more up to 4-fold (on average 2.54-fold higher yields and 2.68-fold higher titers) (FIG. 1B).

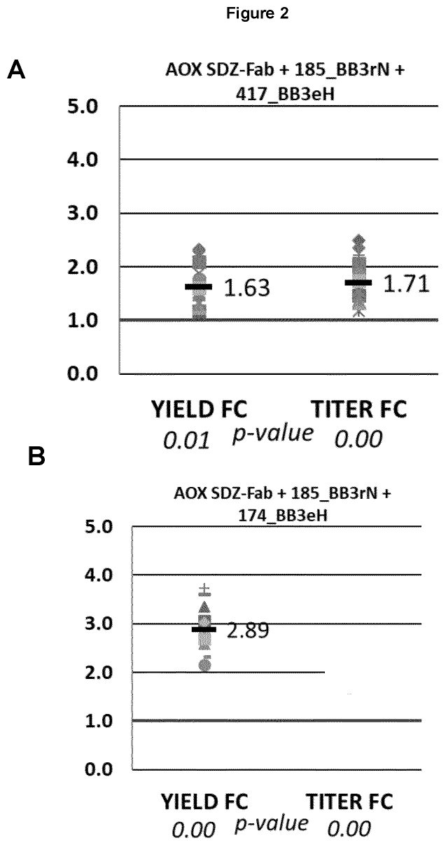

Co-expression of Sbh1 with Lhs1 did not increase Fab secretion compared to overexpression of Sbh1 alone (FIG. 2A), however, co-expression of Sbh1 with Lhs1 and Kar2 enhanced Fab secretion, leading to 2.89-fold higher secreted Fab yields on average (FIG. 2B).

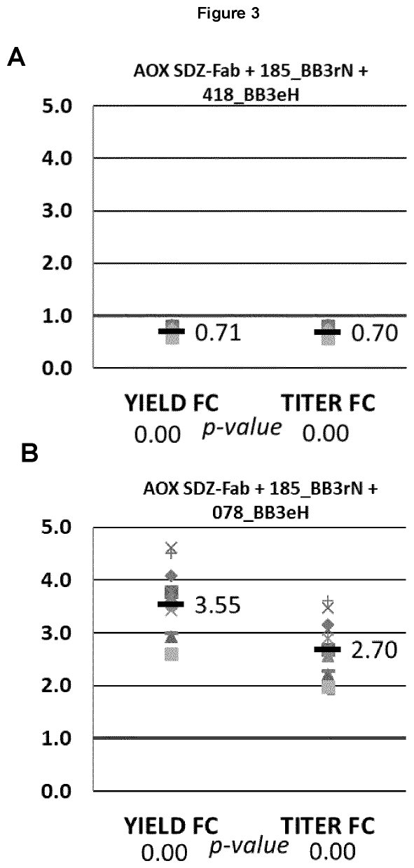

Co-expression of Sbh1 with Sil1 did not increase Fab secretion compared to overexpression of Sbh1 alone (FIG. 3A), however, co-expression of Sbh1 with Sil1 and Kar2 enhanced Fab secretion, leading to 3.55-fold higher secreted Fab yields on average (FIG. 3B).

b) Improvement of scFv Protein Secretion Yields by Overexpression of a Translocation Pore Subunit Alone or in Combination with Helper Gene(s)—Results from Small Scale Screenings.

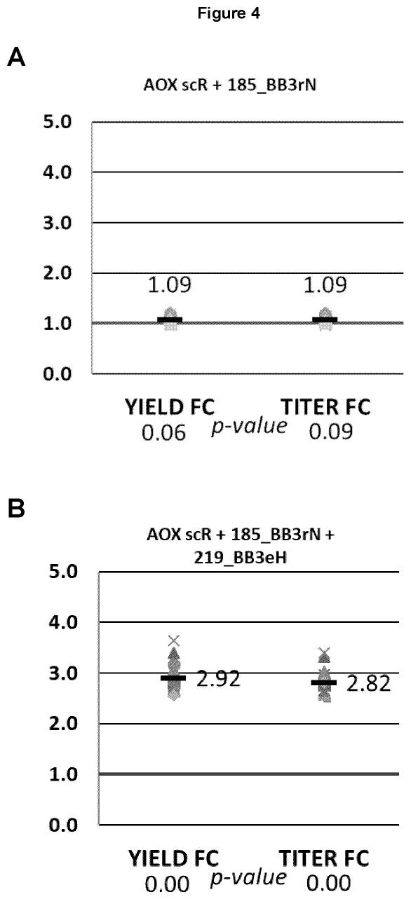

FIGS. 4-6 show the effect of overexpressing sbh1 alone or in combination with helper gene(s) in the scFv producing P. pastoris strain pAOX1-scR #4E3 in small scale screenings (Example 3).

Secretion of scFv is only modestly enhanced (approximately 1.1-fold) by overexpression of the translocon subunit Sbh1 alone (FIG. 4A). As for Fab, scFv secretion yields and titers were further enhanced by combining Sbh1 overexpression with overexpression of chaperones such as Kar2 alone or in combination with Lhs1 or Sil1.

Strikingly, when Sbh1 was co-overexpressed with Kar2, scFv titers and yields were increased up to 3.5-fold (on average 2.92-fold higher yields and 2.82-fold higher titers) (FIG. 4B).

c) Improvement of CES Secretion (Titer and Yield) by Overexpression of a Translocation Pore Subunit Alone or in Combination with Helper Gene(s)—Results from Small Scale Screenings.

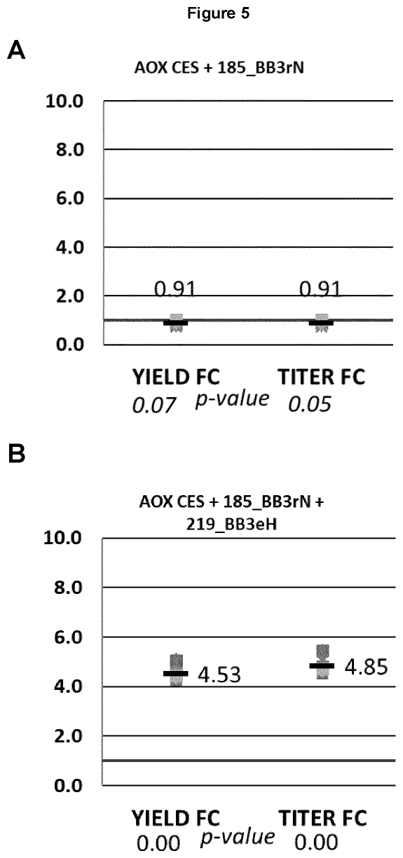

FIGS. 5-7 show the effect of overexpressing sbh1 alone or in combination with helper gene(s) in the carboxylesterase producing P. pastoris strain pAOX1-CES #5 in small scale screenings (Example 3).

Overexpression of Sbh1 did not lead to an enhanced secretion levels of CES (FIG. 5A). Unexpectedly, CES secretion yields and titers were significantly enhanced by combining Sbh1 overexpression with overexpression of Kar2 alone or in combination with Lhs1 or Sil1 nevertheless demonstrating the synergistic effect of Sbh1 with ER chaperones.

Strikingly, when Sbh1 was co-overexpressed with Kar2, CES titers and yields were strongly increased, leading to on average 4.53-fold higher yields and 4.85-fold higher titers (FIG. 5B).

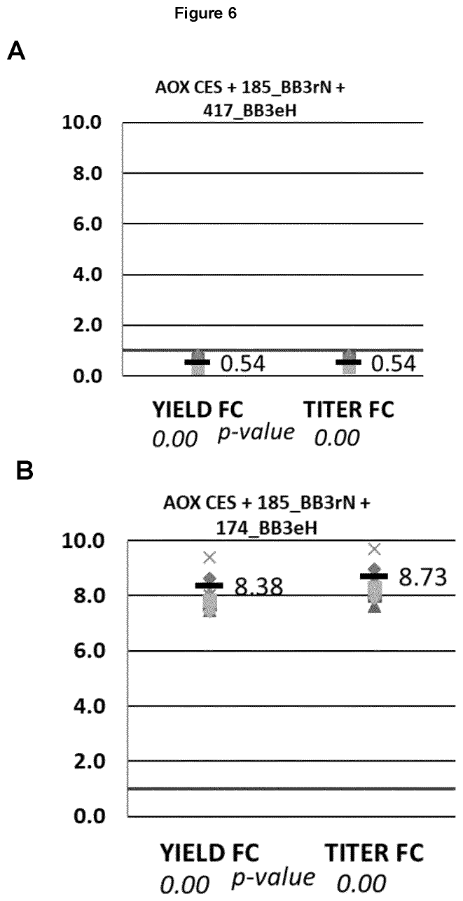

Co-expression of Sbh1 with Lhs1 even lowered CES secretion compared to the parent or overexpression of Sbh1 alone (FIG. 6A). In contrast, co-expression of Sbh1 with Lhs1 and Kar2 markedly enhanced CES secretion, leading to 8-fold higher secreted CES titers and yields on average compared to the parent (FIG. 6B).

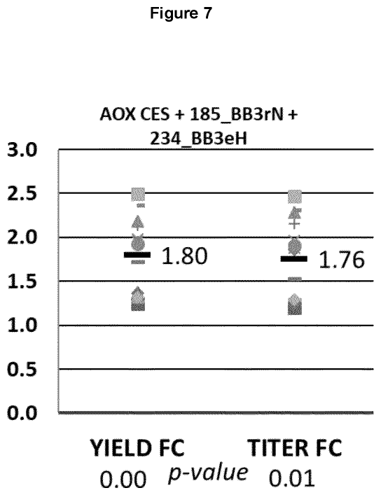

Notably, also the co-overexpression of Sbh1 and the UPR transcription factor Hac1(i) had a beneficial effect on CES secretion, enhancing CES titers and yields up to 2.5-fold (FIG. 7) when compared to just overexpressing Sbh1 alone (FIG. 5A).

### Example 6: Improvement of Recombinant Protein Production and Secretion by Overexpressions of a Translocation Pore Subunit and Helper Gene(s) in Fed Batch Bioreactor Cultivations

Clones of the engineered strains were selected after small scale screening cultivations (Example 5). The selected clones were further evaluated in larger cultivation volumes by fed batch bioreactor cultivations (as described in Example 4). Secretion improvements in small scale screenings, which were also present in fed batch bioreactor cultivations, were verified.

The secretion improvement is measured by titer and yield fold-change values that refer to the respective non-engineered basic production strains (Example 1). The fold-change values of fed batch cultivations are those of single selected clones.

a) Improvement of Fab Protein Secretion Yields by Overexpression of a Translocation Pore Subunit Alone or in Combination with Helper Gene(s)—Results from Fed Batch Bioreactor Cultivations

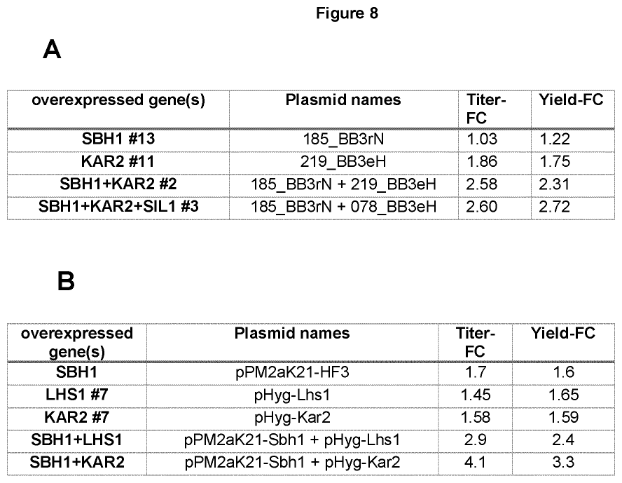

FIG. 8 lists overexpressed genes or gene combinations and their impact on Fab secretion in P. pastoris in fed batch cultivations (Example 4). The positive impact on recombinant protein production observed in screenings were also confirmed in controlled bioreactor cultivations (FIG. 8). As in the screenings, combined overexpression of Sbh1 and chaperones markedly exceeded the performance of strains overexpressing just the latter factors.

After 86 h process time, productivity of Fab (titer and yield) was not or only marginally increased for the strain overexpressing Sbh1, while all co-overexpression strains presented a significantly increased Fab titer and yield. Co-expression of Kar2 alone resulted in 1.82-fold higher titer and 1.74-fold higher yield, which was clearly exceeded for strains co-expressing Sbh1 and Kar2 or Sbh1 and Kar2 and Sil1, respectively (FIG. 8A). When starting from a selected clone containing multiple copies of sbh1-overexpression cassette, already the individual overexpression improved Fab secretion titers and yields (FIG. 8B). The improvements became even more pronounced when Kar2 was co-expressed with Sbh1 in this strain background, leading to 3- to 4-fold increased Fab productivity (titers and yields).

b) Improvement of scFv Protein Secretion Yields by Overexpression of a Translocation Pore Subunit Alone or in Combination with Helper Gene(s)—Results from Fed Batch Bioreactor Cultivations.

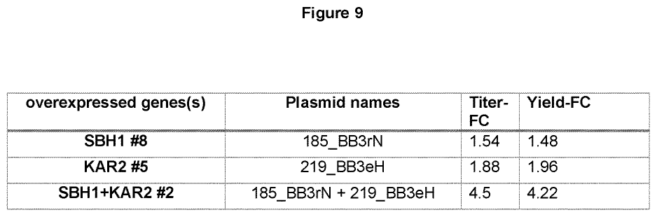

FIG. 9 lists overexpressed genes or gene combinations and their impact on scFv secretion in P. pastoris in fed batch cultivations (Example 4).

Also for the second recombinant model protein, the results obtained in screenings were confirmed under controlled process-like bioreactor conditions (FIG. 9).

After 86 h process time, productivity of scR (titer and yield) was approx. 1.5-fold increased for the strain overexpressing Sbh1 alone, and 2- to 3-fold for strains overexpressing Kar2 alone. Stunningly, the strain co-overexpressing Sbh1 and Kar2 clearly exceeded these two single overexpressions, and presented an even higher Fab productivity, resulting in 4.5-fold higher titer and 4.2-fold higher yield, again highlighting the beneficial effects of combining the overexpression of the translocon subunit Sbh1 with the helper protein Kar2 (FIG. 9).

c) Improvement of CES Protein Secretion Yields by Overexpression of a Translocation Pore Subunit Alone or in Combination with Helper Gene(s)—Results from Fed Batch Bioreactor Cultivations.

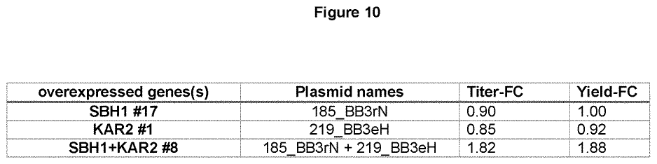

FIG. 10 lists overexpressed genes or gene combinations and their impact on CES secretion in P. pastoris fed batch cultivations (Example 4).

Also for recombinant enzyme model protein, the results obtained in screenings were confirmed under controlled process-like bioreactor conditions (FIG. 10).

As for the other protein producing strains, also in the bioreactor cultivations of CES producing strains, no obvious differences between the parent and the engineered strains were observed regarding growth behavior, biomass accumulation and overall process fitness.

Overexpression of Sbh1 or Kar2 as single factors did not beneficially influence secretion of CES, contrary, productivity of CES (titer and yield) was clearly increased for the strain co-overexpressing Sbh1 and Kar2, leading to 1.8-fold higher titers and yields in the analysed clone (FIG. 10). This clearly emphasizes the synergistic effect observed when co-overexpressing the translocon pore subunit Sbh1 with the helper protein Kar2.

### Items

1. A method of increasing the yield and/or titer of at least one protein of interest in a eukaryotic host cell which comprises at least one polynucleotide encoding the at least one protein of interest, comprising overexpressing in said host cell:
   a) at least one polynucleotide encoding at least one Sbh1 transport protein; and
   b) (i) at least one polynucleotide encoding at least one Kar2 endoplasmic reticulum (ER) helper protein; or
      (ii) at least one polynucleotide encoding at least one Hac1 transcription factor,
   thereby increasing the yield and/or titer of the protein of interest in comparison to a eukaryotic host cell which does not overexpress the polynucleotides of a) and b).
2. The method of item 1, comprising:
   engineering said host cell to overexpress the polynucleotides of a) and b);
   engineering said host cell to comprise at least one polynucleotide encoding the at least one protein of interest, preferably operably linked to a promoter;
   culturing said host cell under suitable conditions to overexpress the polynucleotides of a) and b), and to express the at least one polynucleotide encoding the at least one protein of interest, optionally
   isolating the at least one protein of interest from the cell culture, and optionally
   purifying the at least one protein of interest.
3. A method of manufacturing at least one protein of interest in a eukaryotic host cell comprising:
   providing said host cell engineered to overexpress
   a) at least one polynucleotide encoding at least one Sbh1 transport protein, and
   b) (i) at least one polynucleotide encoding at least one Kar2 endoplasmic reticulum (ER) helper protein; or
   (ii) at least one polynucleotide encoding at least one Hac1 transcription factor,
   wherein the host cell further comprises at least one polynucleotide encoding the at least one protein of interest,
   culturing said host cell under suitable conditions to overexpress the polynucleotides a) and b) and to express the at least one polynucleotide encoding the at least one protein of interest, optionally
   isolating the at least one protein of interest from the cell culture, optionally
   purifying the at least one protein of interest, optionally
   modifying the at least one protein of interest, and optionally
   formulating the at least one protein of interest.
4. The method of any one of the preceding items, wherein the host cell is engineered to overexpress at least one polynucleotide encoding at least one Hac1 transcription factor, thereby enabling overexpression of the at least one Kar2 ER helper protein.
5. The method of any one of the preceding items, wherein the protein of interest is an enzyme, a therapeutic protein, a food additive or a feed additive.
6. The method of item 5, wherein the therapeutic protein comprises an antibody or an antibody fragment comprising at least an antigen-binding site, such as an antibody comprising one or more single variable domains.
7. The method of any one of the preceding items, wherein the overexpression of said polynucleotides of a) and b) increases the yield and/or titer of at least one of the model protein Fab (SEQ ID NOs: 55 and 56), scFv (SEQ ID NO: 57), or CES, in comparison to a host cell which is not engineered to overexpress said polynucleotides of a) and b).
8. The method of any one of the preceding items, wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.
9. The method of any one of the preceding items, wherein at least one of the at least one polynucleotide encoding the at least one protein of interest and the polynucleotides of a) and b) are integrated in the genome of the host cell or contained in a vector or plasmid, which does not integrate into the genome of the host cell.
10. The method of any one of the preceding items, wherein the at least one polynucleotide encoding the at least one Sbh1 transport protein and the at least one polynucleotide encoding the at least one Kar2 ER helper protein or the at least one polynucleotide encoding the at least one Hac1 transcription factor encode for the respective proteins according to one of the following:
   a) a heterologous Sbh1 transport protein and a heterologous Kar2 ER helper protein or a heterologous Hac1 transcription factor;
   b) a homologous Sbh1 transport protein and a homologous Kar2 ER helper protein or a homologous Hac1 transcription factor;
   c) a heterologous Sbh1 transport protein and a homologous Kar2 ER helper protein or a homologous Hac1 transcription factor; or
   d) a homologous Sbh1 protein transport protein and a heterologous Kar2 ER helper protein or a heterologous Hac1 transcription factor.
11. The method of any one of the preceding items, wherein the overexpression of the polynucleotides a) and b) is achieved by
   (i) using a recombinant promoter which drives expression of the polynucleotides;
   (ii) exchanging or modifying a regulatory sequence operably linked to the polynucleotides; or
   (iii) introducing one or more copies of the polynucleotides operably linked to a promoter into the host cell.
12. The method of any one of the preceding items, wherein the overexpression of the polynucleotides a) and b) is achieved by
   i) exchanging the native promoter operably linked to the endogenous polynucleotides by a stronger recombinant promoter;
   ii) exchanging the coding sequence of said endogenous polynucleotides by a codon-optimized coding sequence, which codon-optimization is done according to the codon-usage of said host cell;
   iii) exchanging a native positive regulatory element of the endogenous polynucleotides for a more efficient positive regulatory element;
   iv) introducing a positive regulatory element for the polynucleotides, which element is not present in the respective endogenous expression cassette of the host cell; or
   v) deleting a negative regulatory element of the endogenous polynucleotides, which element is normally present in the respective endogenous expression cassette of the host cell, or a combination thereof.
13. The method of any one of the preceding items, wherein the eukaryotic host cell is a fungal host cell.
14. The method of item 13, wherein the fungal host cell is a yeast host cell.
15. The method of item 14, wherein the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.
16. The method of any one of the preceding items, wherein the Sbh1 transport protein comprises an amino acid sequence as shown in any one of SEQ ID NOs: 1-9 or a functional homolog thereof comprising at least 90% sequence identity to an amino acid sequence as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.
17. The method of any one of the preceding items, wherein the Kar2 ER helper protein comprises an amino acid sequence as shown in any one of SEQ ID NOs: 10-18 or a functional homolog thereof comprising at least 90% sequence identity to an amino acid sequence as shown in any one of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18.
18. The method of any one of the preceding items, wherein the Hac1 transcription factor comprises at least:
   a) a DNA binding domain comprising:
      i) an amino acid sequence as shown in any one of SEQ ID NOs: 19-27, or
      ii) a functional homolog thereof comprising at least 90% sequence identity to an amino acid sequence as shown in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27, and
   b) an activation domain.
19. The method of any one of the preceding items, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.
20. The method of any one of the preceding items, further comprising overexpressing in said host cell or engineering said host cell to overexpress at least one polynucleotide encoding at least one additional ER helper protein.
21. The method of item 20, wherein said at least one additional ER helper protein comprises
   (i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46; or
   (ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.
22. A recombinant eukaryotic host cell for manufacturing at least one protein of interest, which is engineered to overexpress:
   a) at least one polynucleotide encoding at least one Sbh1 transport protein as defined in any one of the preceding items, and
   b) (i) at least one polynucleotide encoding at least one Kar2 endoplasmic reticulum (ER) helper protein as defined in any one of the preceding items, or
      (ii) at least one polynucleotide encoding at least one Hac1 transcription factor as defined in any one of the preceding items.
23. The recombinant eukaryotic host cell of item 22, wherein the host cell is engineered to overexpress at least one polynucleotide encoding at least one Hac1 transcription factor, thereby enabling overexpression of the at least one Kar2 ER helper protein.
24. The recombinant eukaryotic host cell of any one of items 22-23, wherein the protein of interest is an enzyme, a therapeutic protein, a food additive or a feed additive.
25. The recombinant eukaryotic host cell of item 24, wherein the therapeutic protein comprises an antibody or an antibody fragment comprising at least an antigen-binding site, such as an antibody comprising one or more single variable domains.
26. The recombinant eukaryotic host cell of any one of items 22-25, wherein the overexpression of said polynucleotides of a) and b) increases the yield and/or titer of at least any one of the model protein Fab (SEQ ID NOs: 55 and 56), scFv (SEQ ID NO: 57), or CES, in comparison to a host cell which is not engineered to overexpress said polynucleotides of a) and b).
27. The recombinant eukaryotic host cell of item 26, wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.
28. The recombinant eukaryotic host cell of any one of items 22-27, wherein at least one of the at least one polynucleotide encoding the at least one protein of interest and the polynucleotides of a) and b) are integrated in the genome of the host cell or contained in a vector or plasmid, which does not integrate into the genome of the host cell.
29. The recombinant eukaryotic host cell of any one of items 22-28, wherein overexpression of the polynucleotides is achieved by
   (i) using a recombinant promoter which drives expression of the polynucleotides;
   (ii) exchanging or modifying a regulatory sequence operably linked to the polynucleotides; or
   (iii) introducing one or more copies of the polynucleotides operably linked to a promoter into the host cell.
30. The recombinant eukaryotic host cell of any one of items 22-29, wherein overexpression of the polynucleotides is achieved by
   i) exchanging the native promoter operably linked to the endogenous polynucleotides by a stronger recombinant promoter;
   ii) exchanging the coding sequence of said endogenous polynucleotides by a codon-optimized coding sequence, which codon-optimization is done according to the codon-usage of said host cell;
   iii) exchanging a native positive regulatory element of the endogenous polynucleotides for a more efficient positive regulatory element;
   iv) introducing a positive regulatory element for the polynucleotides, which element is not present in the respective endogenous expression cassette of the host cell; or
   v) deleting a negative regulatory element of the endogenous polynucleotides, which element is normally present in the respective endogenous expression cassette of the host cell, or a combination thereof.
31. The recombinant eukaryotic host cell of any one of items 22-30, wherein the eukaryotic host cell is a fungal host cell.
32. The recombinant eukaryotic host cell of item 31, wherein the fungal host cell is a yeast host cell.
33. The recombinant eukaryotic host cell of item 32, wherein the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.
34. The recombinant eukaryotic host cell of any one of items 22-33, wherein the Sbh1 transport protein comprises an amino acid sequence as shown in any one of SEQ ID NOs: 1-9 or a functional homolog thereof comprising at least 90% sequence identity to an amino acid sequence as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.
35. The recombinant eukaryotic host cell of any one of items 22-34, wherein the Kar2 ER helper protein comprises an amino acid sequence as shown in any one of SEQ ID NOs: 10-18 or a functional homolog thereof comprising at least 90% sequence identity to an amino acid sequence as shown in any one of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18.
36. The recombinant eukaryotic host cell of any one of items 22-35, wherein the Hac1 transcription factor comprises at least:
   a) a DNA binding domain comprising:
      i) an amino acid sequence as shown in any one of SEQ ID NOs: 19-27, or
      ii) a functional homolog thereof comprising at least 90% sequence identity to an amino acid sequence as shown in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27, and
   b) an activation domain.
37. The recombinant eukaryotic host cell of any one of items 22-36, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.
38. The recombinant eukaryotic host cell of any one of items 22-37, wherein said host cell is additionally engineered to overexpress at least one polynucleotide encoding at least one additional ER helper protein.
39. The recombinant eukaryotic host cell of item 38, wherein said at least one additional ER helper protein comprises
   (i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46; or
   (ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.
40. Use of the recombinant eukaryotic host cell of any one of items 22-39 for manufacturing at least one protein of interest.

1. A method of increasing the yield and/or titer of at least one protein of interest in a eukaryotic host cell which comprises at least one polynucleotide encoding the at least one protein of interest, comprising overexpressing in said host cell:
a) at least one polynucleotide encoding at least one Sbh1 transport protein; and
b) (i) at least one polynucleotide encoding at least one Kar2 endoplasmic reticulum (ER) helper protein; or
(ii) at least one polynucleotide encoding at least one Hac1 transcription factor,
thereby increasing the yield and/or titer of the protein of interest in comparison to a eukaryotic host cell which does not overexpress the polynucleotides of a) and b).

2. The method of claim 1, comprising:
engineering said host cell to overexpress the polynucleotides of a) and b);
engineering said host cell to comprise at least one polynucleotide encoding the at least one protein of interest, preferably operably linked to a promoter;
culturing said host cell under suitable conditions to overexpress the polynucleotides of a) and b), and to express the at least one polynucleotide encoding the at least one protein of interest, optionally
isolating the at least one protein of interest from the cell culture, and optionally
purifying the at least one protein of interest.

3. A method of manufacturing at least one protein of interest in a eukaryotic host cell comprising:
providing said host cell engineered to overexpress
a) at least one polynucleotide encoding at least one Sbh1 transport protein, and
b) (i) at least one polynucleotide encoding at least one Kar2 endoplasmic reticulum (ER) helper protein; or
(ii) at least one polynucleotide encoding at least one Hac1 transcription factor,
wherein the host cell further comprises at least one polynucleotide encoding the at least one protein of interest,
culturing said host cell under suitable conditions to overexpress the polynucleotides a) and b) and to express the at least one polynucleotide encoding the at least one protein of interest, optionally
isolating the at least one protein of interest from the cell culture, optionally
purifying the at least one protein of interest, optionally
modifying the at least one protein of interest, and optionally
formulating the at least one protein of interest.

4. The method of any one of the preceding claims, wherein the host cell is engineered to overexpress at least one polynucleotide encoding at least one Hac1 transcription factor, thereby enabling overexpression of the at least one Kar2 ER helper protein.

5. The method of any one of the preceding claims, wherein the protein of interest is an enzyme, a therapeutic protein, a food additive or a feed additive.

6. The method of claim 5, wherein the therapeutic protein comprises an antibody or an antibody fragment comprising at least an antigen-binding site, such as an antibody comprising one or more single variable domains.

7. The method of any one of the preceding claims, wherein the overexpression of said polynucleotides of a) and b) increases the yield and/or titer of at least one of the model protein Fab (SEQ ID NOs: 55 and 56), scFv (SEQ ID NO: 57), or CES, in comparison to a host cell which is not engineered to overexpress said polynucleotides of a) and b).

8. The method of any one of the preceding claims, wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.

9. The method of any one of the preceding claims, wherein at least one of the at least one polynucleotide encoding the at least one protein of interest and the polynucleotides of a) and b) are integrated in the genome of the host cell or contained in a vector or plasmid, which does not integrate into the genome of the host cell.

10. The method of any one of the preceding claims, wherein the at least one polynucleotide encoding the at least one Sbh1 transport protein and the at least one polynucleotide encoding the at least one Kar2 ER helper protein or the at least one polynucleotide encoding the at least one Hac1 transcription factor encode for the respective proteins according to one of the following:
a) a heterologous Sbh1 transport protein and a heterologous Kar2 ER helper protein or a heterologous Hac1 transcription factor;
b) a homologous Sbh1 transport protein and a homologous Kar2 ER helper protein or a homologous Hac1 transcription factor;
c) a heterologous Sbh1 transport protein and a homologous Kar2 ER helper protein or a homologous Hac1 transcription factor; or
d) a homologous Sbh1 protein transport protein and a heterologous Kar2 ER helper protein or a heterologous Hac1 transcription factor.

11. The method of any one of the preceding claims, wherein the overexpression of the polynucleotides a) and b) is achieved by
(i) using a recombinant promoter which drives expression of the polynucleotides;
(ii) exchanging or modifying a regulatory sequence operably linked to the polynucleotides; or
(iii) introducing one or more copies of the polynucleotides operably linked to a promoter into the host cell.

12. The method of any one of the preceding claims, wherein the overexpression of the polynucleotides a) and b) is achieved by
i) exchanging the native promoter operably linked to the endogenous polynucleotides by a stronger recombinant promoter;
ii) exchanging the coding sequence of said endogenous polynucleotides by a codon-optimized coding sequence, which codon-optimization is done according to the codon-usage of said host cell;
iii) exchanging a native positive regulatory element of the endogenous polynucleotides for a more efficient positive regulatory element;
iv) introducing a positive regulatory element for the polynucleotides, which element is not present in the respective endogenous expression cassette of the host cell; or
v) deleting a negative regulatory element of the endogenous polynucleotides, which element is normally present in the respective endogenous expression cassette of the host cell, or a combination thereof.

13. The method of any one of the preceding claims, wherein the eukaryotic host cell is a fungal host cell.

14. The method of claim 13, wherein the fungal host cell is a yeast host cell.

15. The method of claim 14, wherein the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.

16. The method of any one of the preceding claims, wherein the Sbh1 transport protein comprises an amino acid sequence as shown in any one of SEQ ID NOs: 1-9 or a functional homolog thereof comprising at least 90% sequence identity to an amino acid sequence as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

17. The method of any one of the preceding claims, wherein the Kar2 ER helper protein comprises an amino acid sequence as shown in any one of SEQ ID NOs: 10-18 or a functional homolog thereof comprising at least 90% sequence identity to an amino acid sequence as shown in any one of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18.

18. The method of any one of the preceding claims, wherein the Hac1 transcription factor comprises at least:
a) a DNA binding domain comprising:
i) an amino acid sequence as shown in any one of SEQ ID NOs: 19-27, or
ii) a functional homolog thereof comprising at least 90% sequence identity to an amino acid sequence as shown in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27, and
b) an activation domain.

19. The method of any one of the preceding claims, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.

20. The method of any one of the preceding claims, further comprising overexpressing in said host cell or engineering said host cell to overexpress at least one polynucleotide encoding at least one additional ER helper protein.

21. The method of claim 20, wherein said at least one additional ER helper protein comprises
(i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46; or
(ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

22. A recombinant eukaryotic host cell for manufacturing at least one protein of interest, which is engineered to overexpress:
a) at least one polynucleotide encoding at least one Sbh1 transport protein as defined in any one of the preceding claims, and
b) (i) at least one polynucleotide encoding at least one Kar2 endoplasmic reticulum (ER) helper protein as defined in any one of the preceding claims, or
(ii) at least one polynucleotide encoding at least one Hac1 transcription factor as defined in any one of the preceding claims.

23. The recombinant eukaryotic host cell of claim 22, wherein the host cell is engineered to overexpress at least one polynucleotide encoding at least one Hac1 transcription factor, thereby enabling overexpression of the at least one Kar2 ER helper protein.

24. The recombinant eukaryotic host cell of any one of claims 22-23, wherein the protein of interest is an enzyme, a therapeutic protein, a food additive or a feed additive.

25. The recombinant eukaryotic host cell of claim 24, wherein the therapeutic protein comprises an antibody or an antibody fragment comprising at least an antigen-binding site, such as an antibody comprising one or more single variable domains.

26. The recombinant eukaryotic host cell of any one of claims 22-25, wherein the overexpression of said polynucleotides of a) and b) increases the yield and/or titer of at least any one of the model protein Fab (SEQ ID NOs: 55 and 56), scFv (SEQ ID NO: 57), or CES, in comparison to a host cell which is not engineered to overexpress said polynucleotides of a) and b).

27. The recombinant eukaryotic host cell of claim 26, wherein the yield and/or titer of the at least one protein of interest is increased at least about 1.1-fold, at least about 1.25-fold, at least about 1.5-fold, at least about 2-fold, at least about 3-fold, at least about 4-fold, at least about 5-fold, at least about 7.5-fold, or at least about 10-fold.

28. The recombinant eukaryotic host cell of any one of claims 22-27, wherein at least one of the at least one polynucleotide encoding the at least one protein of interest and the polynucleotides of a) and b) are integrated in the genome of the host cell or contained in a vector or plasmid, which does not integrate into the genome of the host cell.

29. The recombinant eukaryotic host cell of any one of claims 22-28, wherein overexpression of the polynucleotides is achieved by
(i) using a recombinant promoter which drives expression of the polynucleotides;
(ii) exchanging or modifying a regulatory sequence operably linked to the polynucleotides; or
(iii) introducing one or more copies of the polynucleotides operably linked to a promoter into the host cell.

30. The recombinant eukaryotic host cell of any one of claims 22-29, wherein overexpression of the polynucleotides is achieved by
i) exchanging the native promoter operably linked to the endogenous polynucleotides by a stronger recombinant promoter;
ii) exchanging the coding sequence of said endogenous polynucleotides by a codon-optimized coding sequence, which codon-optimization is done according to the codon-usage of said host cell;
iii) exchanging a native positive regulatory element of the endogenous polynucleotides for a more efficient positive regulatory element;
iv) introducing a positive regulatory element for the polynucleotides, which element is not present in the respective endogenous expression cassette of the host cell; or
v) deleting a negative regulatory element of the endogenous polynucleotides, which element is normally present in the respective endogenous expression cassette of the host cell, or a combination thereof.

31. The recombinant eukaryotic host cell of any one of claims 22-30, wherein the eukaryotic host cell is a fungal host cell.

32. The recombinant eukaryotic host cell of claim 31, wherein the fungal host cell is a yeast host cell.

33. The recombinant eukaryotic host cell of claim 32, wherein the yeast host cell is selected from the group consisting of Komagataella phaffii, Hansenula polymorpha, Trichoderma reesei, Saccharomyces cerevisiae, Kluyveromyces lactis, Yarrowia lipolytica, Pichia methanolica, Candida boidinii, and Schizosaccharomyces pombe.

34. The recombinant eukaryotic host cell of any one of claims 22-33, wherein the Sbh1 transport protein comprises an amino acid sequence as shown in any one of SEQ ID NOs: 1-9 or a functional homolog thereof comprising at least 90% sequence identity to an amino acid sequence as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 9.

35. The recombinant eukaryotic host cell of any one of claims 22-34, wherein the Kar2 ER helper protein comprises an amino acid sequence as shown in any one of SEQ ID NOs: 10-18 or a functional homolog thereof comprising at least 90% sequence identity to an amino acid sequence as shown in any one of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17 or SEQ ID NO: 18.

36. The recombinant eukaryotic host cell of any one of claims 22-35, wherein the Hac1 transcription factor comprises at least:
a) a DNA binding domain comprising:
i) an amino acid sequence as shown in any one of SEQ ID NOs: 19-27, or
ii) a functional homolog thereof comprising at least 90% sequence identity to an amino acid sequence as shown in any one of SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 or SEQ ID NO: 27, and
b) an activation domain.

37. The recombinant eukaryotic host cell of any one of claims 22-36, wherein the Hac1 transcription factor comprises an amino acid sequence as shown in any one of SEQ ID NOs: 28-36 or a functional homolog thereof comprising at least 90% sequence identity to an amino acid sequence as shown in any one of SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35 or SEQ ID NO: 36.

38. The recombinant eukaryotic host cell of any one of claims 22-37, wherein said host cell is additionally engineered to overexpress at least one polynucleotide encoding at least one additional ER helper protein.

39. The recombinant eukaryotic host cell of claim 38, wherein said at least one additional ER helper protein comprises
(i) Lhs1 helper protein, preferably comprising an amino acid sequence as shown in any one of SEQ ID NOs: 37-46 or a functional homolog thereof comprising at least 90% sequence identity to an amino acid sequence as shown in any one of SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 or SEQ ID NO: 46; or
(ii) Sil1 helper protein, preferably comprising an amino acid sequence as shown in any one of SEQ ID NOs: 47-54 or a functional homolog thereof comprising at least 90% sequence identity to an amino acid sequence as shown in any one of SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

40. Use of the recombinant eukaryotic host cell of any one of claims 22-39 for manufacturing at least one protein of interest.


SEQUENCE LISTING (88 sequences)

SEQ ID NO: 1 - protein, 86 aa, Komagataella phaffii
MSTAIPGGQRTLAKRRAANLDKKQDEPTSARSAGAGGSSSTMLKLYTDEAQGLKVDPLIVLVLAVGFIFSVIGLHVVAKLTGKLIN

SEQ ID NO: 2 - protein, 88 aa, Kluyveromyces lactis
MDSSVPGGQRTLQKRRNAQLQKEKKANQTPASPRQAGFGGSSSSILKLYTDEANGLRVDPLVVLFLAVAFVFSVVALHVVAKVSGKIF

SEQ ID NO: 3 - protein, 91 aa, Yarrowia lipolytica
MSTSAQVPGGPAAQMKRRNNAQRQEAKASQRPTSTRSVGAGGSSSTMLKLYTDESQGLKVDPVVVMVLSLGFIFSVVALHILAKVSTKLLG

SEQ ID NO: 4 - protein, 111 aa, Ogataea polymorpha
MATYQSSAAGVVWRSAVCVSTIRQPHLAMKLLTLAKRKATQENKLKQQLQSQPTSTRAAGAGGSSSTMLKIYTDESQGFKIDPLVVLVLAVSFIFSVVLLHVLAKLTGKFF

SEQ ID NO: 5 - protein, 82 aa, Saccharomyces cerevisiae
MSSPTPPGGQRTLQKRKQGSSQKVAASAPKKNTNSNNSILKIYSDEATGLRVDPLVVLFLAVGFIFSVVALHVISKVAGKLF

SEQ ID NO: 6 - protein, 88 aa, Saccharomyces cerevisiae
MAASVPPGGQRILQKRRQAQSIKEKQAKQTPTSTRQAGYGGSSSSILKLYTDEANGFRVDSLVVLFLSVGFIFSVIALHLLTKFTHII

SEQ ID NO: 7 - protein, 127 aa, Aspergillus niger
MASPRAASPMTSGAESGPDSKSAGAGTGASAVSSVNRPSSPTPPGGPRAAMRRRAAADHKESLRNARPSSTRAAGAGGSSGTMLKLYTDESPGLRVDPVVVLVLSLGFIFSVVGLHVIAKITRKFSS

SEQ ID NO: 8 - protein, 112 aa, Trichoderma reesei
MSSRPSSPANGPAAASGSSISRPSSPTPPGGPRTAIRRRAAADQKEKLANARPSSTRAAGAGGSSSTMLRLYTDESPGLKVDPVVVLVLSLVFIFSVVALHIIAKISRKFSS

SEQ ID NO: 9 - protein, 102 aa, Schizosaccharomyces pombe
MSSTKASGSVKNSAASAPGGPKSQIRRRAAVEKNTKESNSGPAGARAAGAPGSTPTLLKLYTDEASGFKVDPVVVMVLSVGFIASVFLLHIVARILKKFASE

SEQ ID NO: 10 - protein, 678 aa, Komagataella phaffii
MLSLKPSWLTLAALMYAMLLVVVPFAKPVRADDVESYGTVIGIDLGTTYSCVGVMKSGRVEILANDQGNRITPSYVSFTEDERLVGDAAKNLAASNPKNTIFDIKRLIGMKYDAPEVQRDLKRLPYTVKSKNGQPVVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISNNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDYFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGTPPGGQGFDDDDGDFDYDYDYDHDEL

SEQ ID NO: 11 - protein, 677 aa, Komagataella pastoris
MQSLKPSWLTLAALLYAMLMVVVPFAKPVRADDVESYGTVIGIDLGTTYSCVGVMKSGRVEILANDQGNRITPSYVSFTEDERLVGDAAKNLAASNPKNTIFDIKRLIGMKFDSPEVQRDLKRLPYSVKSKNGQPIVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISDNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDFFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGAPPGQGFDDDDGDFDYDYDYDHDEL

SEQ ID NO: 12 - protein, 670 aa, Yarrowia lipolytica
MKFSMPSWGVVFYALLVCLLPFLSKAGVQADDVDSYGTVIGIDLGTTYSCVGVMKGGRVEILANDQGSRITPSYVAFTEDERLVGDAAKNQAANNPFNTIFDIKRLIGLKYKDESVQRDIKHFPYKVKNKDGKPVVVVETKGEKKTYTPEEISAMILTKMKDIAQDYLGKKVTHAVVTVPAYFNDAQRQATKDAGIIAGLNVLRIVNEPTAAAIAYGLDHTDDEKQIVVYDLGGGTFDVSLLSIESGVFEVLATAGDTHLGGEDFDYRVIKHFVKQYNKKHDVDITKNAKTIGKLKREVEKAKRTLSSQMSTRIEIESFFDGEDFSETLTRAKFEELNIDLFKRTLKPVEQVLKDSGVKKEDVHDIVLVGGSTRIPKVQELLEKFFDGKKASKGINPDEAVAYGAAVQAGVLSGEDGVEDIVLLDVNPLTLGIETTGGVMTKLINRNTNIPTKKSQIFSTAVDNQSTVLIQVFEGERTMSKDNNLLGKFELKGIPPAPRGVPQIEVTFELDANGILRVTAHDKGTGKSETITITNDKGRLSKDEIERMVEEAERFAEEDALIRETIEAKNSLENYAHSLRNQVADKSGLGGKISADDKEALNDAVTETLEWLEANSVSATKEDFEEKKEALSAIAYPITSKIYEGGEGGDESNDGGFYADDDEAPFHDEL

SEQ ID NO: 13 - protein, 682 aa, Saccharomyces cerevisiae
MFFNRLSAGKLLVPLSVVLYALFVVILPLQNSFHSSNVLVRGADDVENYGTVIGIDLGTTYSCVAVMKNGKTEILANEQGNRITPSYVAFTDDERLIGDAAKNQVAANPQNTIFDIKRLIGLKYNDRSVQKDIKHLPFNVVNKDGKPAVEVSVKGEKKVFTPEEISGMILGKMKQIAEDYLGTKVTHAVVTVPAYFNDAQRQATKDAGTIAGLNVLRIVNEPTAAAIAYGLDKSDKEHQIIVYDLGGGTFDVSLLSIENGVFEVQATSGDTHLGGEDFDYKIVRQLIKAFKKKHGIDVSDNNKALAKLKREAEKAKRALSSQMSTRIEIDSFVDGIDLSETLTRAKFEELNLDLFKKTLKPVEKVLQDSGLEKKDVDDIVLVGGSTRIPKVQQLLESYFDGKKASKGINPDEAVAYGAAVQAGVLSGEEGVEDIVLLDVNALTLGIETTGGVMTPLIKRNTAIPTKKSQIFSTAVDNQPTVMIKVYEGERAMSKDNNLLGKFELTGIPPAPRGVPQIEVTFALDANGILKVSATDKGTGKSESITITNDKGRLTQEEIDRMVEEAEKFASEDASIKAKVESRNKLENYAHSLKNQVNGDLGEKLEEEDKETLLDAANDVLEWLDDNFETAIAEDFDEKFESLSKVAYPITSKLYGGADGSGAADYDDEDEDDDGDYFEHDEL

SEQ ID NO: 14 - protein, 665 aa, Ogataea polymorpha
MLTFNKSVVSCAAIIYALLLVVLPLTTQQFVKAESNENYGTVIGIDLGTTYSCVGVMKAGRVEIIPNDQGNRITPSYVAFTEDERLVGDAAKNQIASNPTNTIFDIKRLIGHRFDDKVIQKEIKHLPYKVKDQDGRPVVEAKVNGELKTFTAEEISAMILGKMKQIAEDYLGKKVTHAVVTVPAYFNDAQRQATKDAGTIAGLEVLRIVNEPTAAAIAYGLDKTDEEKHIIVYDLGGGTFDVSLLTIAGGAFEVLATAGDTHLGGEDFDYRVVRHFIKVFKKKHGIDISDNSKALAKLKREVEKAKRTLSSQMSTRIEIDSFVDGIDFSESLSRAKFEELNMDLFKKTLKPVQQVLDDAKMKPDEIDDVVFVGGSTRIPKVQELIENFFNGKKISKGINPDEAVAFGAAVQGGVLSGEEGVEDIVLIDVNPLTLGIETSGGVMTTLIKRNTPIPTQKSQIFSTAADNQPVVLIQVYEGERAMAKDNNLLGKFELTGIPPAPRGVPQIEVTFTLDSNGILKVSATDKGTGKSNSITITNDKGRLSKEEIEKKIEEAEKFAQQDKELREKVESRNALENYAHSLKNQANDENGFGAKLEEDDKETLLDAINEALEFLEDNFDTATKDEFDEQKEKLSKVAYPITSKLYDAPPTSDEEDEDDWDHDEL

SEQ ID NO: 15 - protein, 664 aa, Trichoderma reesei
MARSRSSLALGLGLLCWITLLFAPLAFVGKANAASDDADNYGTVIGIDLGTTYSCVGVMQKGKVEILVNDQGNRITPSYVAFTDEERLVGDSAKNQAAANPTNTVYDVKRLIGRKFDEKEIQADIKHFPYKVIEKNGKPVVQVQVNGQKKQFTPEEISAMILGKMKEVAESYLGKKVTHAVVTVPAYFNDNQRQATKDAGTIAGLNVLRIVNEPTAAAIAYGLDKTDGERQIIVYDLGGGTFDVSLLSIDNGVFEVLATAGDTHLGGEDFDQRIINYLAKAYNKKNNVDISKDLKAMGKLKREAEKAKRTLSSQMSTRIEIEAFFEGNDFSETLTRAKFEELNMDLFKKTLKPVEQVLKDANVKKSEVDDIVLVGGSTRIPKVQSLIEEYFNGKKASKGINPDEAVAFGAAVQAGVLSGEEGTDDIVLMDVNPLTLGIETTGGVMTKLIPRNTPIPTRKSQIFSTAADNQPVVLIQVFEGERSMTKDNNLLGKFELTGIPPAPRGVPQIEVSFELDANGILKVSAHDKGTGKQESITITNDKGRLTQEEIDRMVAEAEKFAEEDKATRERIEARNGLENYAFSLKNQVNDEEGLGGKIDEEDKETILDAVKEATEWLEENGADATTEDFEEQKEKLSNVAYPITSKMYQGAGGSEDDGDFHDEL

SEQ ID NO: 16 - protein, 670 aa, Candida boidinii
MLKFNRSFIASLAILYSLLLIIVPLLSQQAHAEDEHETYGTVIGIDLGTTYSCVGVMKSGKVEILANDQGNRITPSYVAFTDEERLVGDAAKNQAPSNPHNTIFDIKRLIGHSYSDKVVQTEKKHLPYNIIEKQGKPAVEVKFQNELKVFTPEEISSMILGKMKQIAEDYLGKKVTHAVVTVPAYFNDAQRQATKDAGTIAGLNVLRIVNEPTAAAIAYGLDKEGERQIIVYDLGGGTFDVSLLAIENGVFEVLSTSGDTHLGGEDFDFRVVRHFSKIFKKKHNIDISDNAKAISKLKREVEKAKRTLSTQMSTRIEIDSFVDGIDFSETLSRAKFEEINIELFKKTLKPVQQVLDDAGLKAAEIDDIVLVGGSTRIPKVQEILENFFSGKKATKGINPDEAVAYGAAVQAGILSGSEGASDVVLIDVNPLTLGIETTGNVMTTLIKRNTPIPTKKTQVFSTAVDNQDTVLIKVYEGERAMSTDNNLLGSFELKGIPPAPKGSPQIEVTFSLDVNGILRVSATDKSTGKSNSITISNDHGRLSKEEIDKMVEDGEKYAEQDKLFREKIEAKNDLEKYALGLKTQLADESVAEKLAEDEIETVLDAVKEALEFIDENEDATTEDYSEQKEKLIKIASPITTKLFMQPQGGESADEDDEDFDDDYDYGHDEL

SEQ ID NO: 17 - protein, 672 aa, Aspergillus niger
MARISHQGAAKPFTAWTTIFYLLLVFIAPLAFFGTAHAQDETSPQESYGTVIGIDLGTTYSCVGVMQNGKVEILVNDQGNRITPSYVAFTDEERLVGDAAKNQYAANPRRTIFDIKRLIGRKFDDKDVQKDAKHFPYKVVNKDGKPHVKVDVNQTPKTLTPEEVSAMVLGKMKEIAEGYLGKKVTHAVVTVPAYFNDAQRQATKDAGTIAGLNVLRVVNEPTAAAIAYGLDKTGDERQVIVYDLGGGTFDVSLLSIDNGVFEVLATAGDTHLGGEDFDQRVMDHFVKLYNKKNNVDVTKDLKAMGKLKREVEKAKRTLSSQMSTRIEIEAFHNGEDFSETLTRAKFEELNMDLFKKTLKPVEQVLKDAKVKKSEVDDIVLVGGSTRIPKVQALLEEFFGGKKASKGINPDEAVAFGAAVQGGVLSGEEGTGDVVLMDVNPLTLGIETTGGVMTKLIPRNTVIPTRKSQIFSTAADNQPTVLIQVYEGERSLTKDNNLLGKFELTGIPPAPRGVPQIEVSFDLDANGILKVHASDKGTGKAESITITNDKGRLSQEEIDRMVAEAEEFAEEDKAIKAKIEARNTLENYAFSLKNQVNDENGLGGQIDEDDKQTILDAVKEVTEWLEDNAATATTEDFEEQKEQLSNVAYPITSKLYGSAPADEDDEPSGHDEL

SEQ ID NO: 18 - protein, 679 aa, Kluyveromyces lactis
MFSARKSSVGWLVSSLAVFYVLLAVIMPIALTGSQSSRVVARAAEDHEDYGTVIGIDLGTTYSCVAVMKNGKTEILANEQGNRITPSYVSFTDDERLIGDAAKNQAASNPKNTIFDIKRLIGLQYNDPTVQRDIKHLPYTVVNKGNKPYVEVTVKGEKKEFTPEEVSGMILGKMKQIAEDYLGKKVTHAVVTVPAYFNDAQRQATKDAGAIAGLNILRIVNEPTAAAIAYGLDKTEDEHQIIVYDLGGGTFDVSLLSIENGVFEVQATAGDTHLGGEDFDYKLVRHFAQLFQKKHDLDVTKNDKAMAKLKREAEKAKRSLSSQTSTRIEIDSFFNGIDFSETLTRAKFEELNLALFKKTLKPVEKVLKDSGLQKEDIDDIVLVGGSTRIPKVQQLLEKFFNGKKASKGINPDEAVAYGAAVQAGVLSGEEGVEDIVLLDVNALTLGIETTGGVMTPLIKRNTAIPTKKSQIFSTAVDNQKAVRIQVYEGERAMVKDNNLLGNFELSDIRAAPRGVPQIEVTFALDANGILTVSATDKDTGKSESITIANDKGRLSQDDIDRMVEEAEKYAAEDAKFKAKSEARNTFENFVHYVKNSVNGELAEIMDEDDKETVLDNVNESLEWLEDNSDVAEAEDFEEKMASFKESVEPILAKASASQGSTSGEGFEDEDDDDYFDDEL

SEQ ID NO: 19 - protein, 53 aa, Komagataella phaffii
RRVERILRNRRAAHASREKKRRHVEFLENHVVDLESALQESAKATNKLKEIQD

SEQ ID NO: 20 - protein, 53 aa, Komagataella pastoris
RRVERILRNRRAAHASREKKRRHVEFLENHVVDLESALQESAKATNKLKQIQD

SEQ ID NO: 21 - protein, 39 aa, synthetic construct
  REGION 1..39: note = DNA binding domain O. angusta
RRVERILRNRRAAHASREKKRRHVEYLENYVTDLESALA

SEQ ID NO: 22 - protein, 48 aa, Candida boidinii
RRVERILRNRRAAHASREKKRKHVEYLELYVNNLENGIKNYISNQEKL

SEQ ID NO: 23 - protein, 45 aa, Yarrowia lipolytica
RRIERIMRNRQAAHASREKKRRHLEDLEKKCSELSSENNDLHHQV

SEQ ID NO: 24 - protein, 46 aa, Kluyveromyces lactis
RRIERILRNRRAAHQSREKKRLHVQRLEEKCHLLEGILKMVDLDIL

SEQ ID NO: 25 - protein, 51 aa, Saccharomyces cerevisiae
RRIERILRNRRAAHQSREKKRLHLQYLERKCSLLENLLNSVNLEKLADHED

SEQ ID NO: 26 - protein, 53 aa, Trichoderma reesei
RRVERVLRNRRAAQSSRERKRLEVEALEKRNKELETLLINVQKTNLILVEELN

SEQ ID NO: 27 - protein, 53 aa, Aspergillus niger
RRIERVLRNRAAAQTSRERKRLEMEKLENEKIQMEQQNQFLLQRLSQMEAENN

SEQ ID NO: 28 - protein, 331 aa, Komagataella phaffii
MPVDSSHKTASPLPPRKRAKTEEEKEQRRVERILRNRRAAHASREKKRRHVEFLENHVVDLESALQESAKATNKLKEIQDIIVSRLEALGGTVSDLDLTVPEVDFPKSSDLEPMSDLSTSSKSEKASTSTRRSLTEDLDEDDVAEYDDEEEDEELPRKMKVLNDKNKSTSIKQEKLNELPSPLSSDFSDVDEEKSTLTHLKLQQQQQQPVDNYVSTPLSLPEDSVDFINPGNLKIESDENFLLSSNTLQIKHENDTDYITTAPSGSINDFFNSYDISESNRLHHPAVMTDSSLHITAGSIGFFSLIGGGESSVAGRRSSVGTYQLTCIAIR

SEQ ID NO: 29 - protein, 330 aa, Komagataella pastoris
MPVDSSHKIASPLPPRKRAKTEEEKEQRRVERILRNRRAAHASREKKRRHVEFLENHVVDLESALQESAKATNKLKQIQDIIVSRLEALGGTVSDLDLAVPEVDFPKFSDLELSTDLSSSTKSEKASTSTCRSSTEDLDEDGVAEYDDEEDEELPRKKNVLNDKSKNRTIKQEKLNELPSPLSSDFSDVDEEKSTLTHFQLQQQQQQQPVDNYVSTPLSLPEDSIDFINPGSLKIESDENFLLGSSTLQIKHENDTEYIPTAPSGSINDFFNSYDISESNRLHHPAVMTDSSLHTTAGSIGFFSLIRGKSFVVGRRSSVGVYQLTCIAIR

SEQ ID NO: 30 - protein, 325 aa, Ogataea angusta
MTALNSSVQHQEVSSDLPFGTLPPRKRAKTEEEKEQRRVERILRNRRAAHASREKKRRHVEYLENYVTDLESALATHEGNYRKMAKIQSSLISLLSEHGIDYSSVDLAVEPCPKVERPEGLELTGSIPVKKQKIASAKSPKSLSRKSKSEIPSPSFDENIFSEEENEHDDGIEEYGKAGQEATEAPSLSHNRKRKAQDAYISPPGSTSPSKLKLEEDERISKHEYSNLFDDTDDIFPSEKSSSLELYKQDDLTMASFVKQEEEEMVPFVKQEDEFKFPDSGFNADDCHLIQVEDLCSFNSVHHPAAAPLTAESIDNHFEFDDYLS

SEQ ID NO: 31 - protein, 351 aa, Candida boidinii
MSLSNTPSSPDNISNVSASLISSNLKGKTDELLKSASAIGLLPPRKRAKTAEEKEQRRVERILRNRRAAHASREKKRKHVEYLELYVNNLENGIKNYISNQEKLINFQSLLIAKLKVANVDISDIDLSTCTNIDIVSIEKPECLNYSPNSSSKKNKKSSSDDEEEEDDDDDDEDDEDDNVELKHKSNSQKQQQQQQKEYKEVEQSTKQDESKTSNQQQEQEQEQEQVSTPKAELTQQLSDPTMDMKFKSAVKLEDVNQLPQDQYLMSPPNTESPRKFILDSSNINKDYTHIFVGDDLLFNNDLQLCSDSLKQQELNVPNIENIISDYSLDSMNDLNAYNRLHHPAAMVQRY

SEQ ID NO: 32 - protein, 299 aa, Yarrowia lipolytica
MSIKREESFTPTPEDLGSPLTADSPGSPESGDKRKKDLTLPLPAGALPPRKRAKTENEKEQRRIERIMRNRQAAHASREKKRRHLEDLEKKCSELSSENNDLHHQVTESKKTNMHLMEQHYSLVAKLQQLSSLVNMAKSSGALAGVDVPDMSDVSMAPKLEMPTAAPSQPMGLASAPTLFNHDNETVVPDSPIVKTEEVDSTNFLLHTESSSPPELAESTGSGSPSSTLSCDETDYLVDRARHPAVMTVATTDQQRRHKISFSSRTSPLTTSLDCMDCRMTSPCLKTTSSLPSTTLLLI

SEQ ID NO: 33 - protein, 273 aa, Kluyveromyces lactis
MTGKNSVSDIPVNFKPTLPPRKRAKTQEEKEQRRIERILRNRRAAHQSREKKRLHVQRLEEKCHLLEGILKMVDLDILSENNAKLSGMVEQWREMQVSDSGSISSHDSNTGMLDSPESLTSSPDKKDHYSHSSHSTSISSSSSSSSPSNLPHGMVTDNGMLDEDNNSLNYILGQQNYQLSSTPVVKLEEDHSMLLENNGDADLNDVGISFIAEDGTNSDNKNIDMRNQETGEGWNLLLTVPPELNSDLSELEPSDIISPIGLDTWRNPAVIVT

SEQ ID NO: 34 - protein, 238 aa, Saccharomyces cerevisiae
MEMTDFELTSNSQSNLAIPTNFKSTLPPRKRAKTKEEKEQRRIERILRNRRAAHQSREKKRLHLQYLERKCSLLENLLNSVNLEKLADHEDALTCSHDAFVASLDEYRDFQSTRGASLDTRASSHSSSDTFTPSPLNCTMEPATLSPKSMRDSASDQETSWELQMFKTENVPESTTLPAVDNNNLFDAVASPLADPLCDDIAGNSLPFDNSIDLDNWRNPEAQSGLNSFELNDFFITS

SEQ ID NO: 35 - protein, 451 aa, Trichoderma reesei
MAFQQSSPLVKFEASPAESFLSAPGDNFTSLFADSTPSTLNPRDMMTPDSVADIDSRLSVIPESQDAEDDESHSTSATAPSTSEKKPVKKRKSWGQVLPEPKTNLPPRKRAKTEDEKEQRRVERVLRNRRAAQSSRERKRLEVEALEKRNKELETLLINVQKTNLILVEELNRFRRSSGVVTRSSSPLDSLQDSITLSQQLFGSRDGQTMSNPEQSLMDQIMRSAANPTVNPASLSPSLPPISDKEFQTKEEDEEQADEDEEMEQTWHETKEAAAAKEKNSKQSRVSTDSTQRPAVSIGGDAAVPVFSDDAGANCLGLDPVHQDDGPFSIGHSFGLSAALDADRYLLESQLLASPNASTVDDDYLAGDSAACFTNPLPSDYDFDINDFLTDDANHAAYDIVAASNYAAADRELDLEIHDPENQIPSRHSIQQPQSGASSHGCDDGGIAVGV

SEQ ID NO: 36 - protein, 342 aa, Aspergillus niger
MMEEAFSPVDSLAGSPTPELPLLTVSPADTSLDDSSVQAGETKAEEKKPVKKRKSWGQELPVPKTNLPPRKRAKTEDEKEQRRIERVLRNRAAAQTSRERKRLEMEKLENEKIQMEQQNQFLLQRLSQMEAENNRLNQQVAQLSAEVRGSRGNTPKPGSPVSASPTLTPTLFKQERDEIPLERIPFPTPSITDYSPTLRPSTLAESSDVTQHPAVSVAGLEGEGSALSLFDVGSNPEPHAADDLAAPLSDDDFHRLFNVDSPVGSDSSVLEDGFAFDVLDGGDLSAFPFDSMVDFDPESVGFEGIEPPHGLPDETSRQTSSVQPSLGASTSRCDGQGIAAGC

SEQ ID NO: 37 - protein, 894 aa, Komagataella phaffii
MRTQKIVTVLCLLLNTVLGALLGIDYGQEFTKAVLVAPGVPFEVILTPDSKRKDNSMMAIKENSKGEIERYYGSSASSVCIRNPETCLNHLKSLIGVSIDDVSTIDYKKYHSGAEMVPSKNNRNTVAFKLGSSVYPVEEILAMSLDDIKSRAEDHLKHAVPGSYSVISDAVITVPTFFTQSQRLALKDAAEISGLKVVGLVDDGISVAVNYASSRQFNGDKQYHMIYDMGAGSLQATLVSISSSDDGGIVIDVEAIAYDKSLGGQLFTQSVYDILLQKFLSEHPSFSESDFNKNSKSMSKLWQAAEKAKTILSANTDTRVSVESLYNDIDFRATIARDEFEDYNAEHVHRITAPIIEALSHPLNGNLTSPFPLTSLSSVILTGGSTRVPMVKKHLESLLGSELIAKNVNADESAVFGSTLRGVTLSQMFKAKQMTVNERSVYDYCLKVGSSEINVFPVGTPLATKKVVELENVDSENQLTIGLYENGQLFASHEVTDLKKSIKSLTQEGKECSNINYEATVELSESRLLSLTRLQAKCADEAEYLPPVDTESEDTKSENSTTSETIEKPNKKLFYPVTIPTQLKSVHVKPMGSSTKVSSSLKIKELNKKDAVKRSIEELKNQLESKLYRVRSYLEDEEVVEKGPASQVEALSTLVAENLEWLDYDSDDASAKDIREKLNSVSDSVAFIKSYIDLNDVTFDNNLFTTIYNTTLNSMQNVQELMLNMSEDALSLMQQYEKEGLDFAKESQKIKIKSPPLSDKELDNLFNTVTEKLEHVRMLTEKDTISDLPREELFKLYQELQNYSSRFEAIMASLEDVHSQRINRLTDKLRKHIERVSNEALKAALKEAKRQQEEEKSHEQNEGEEQSSASTSHTNEDIEEPSESPKVQTSHDEL

SEQ ID NO: 38 - protein, 880 aa, Komagataella pastoris
MKTQKIVTLLCLLLSNVLGALLGIDYGQEFTKAVLVAPGVPFEVILTPDSKRKDNSMMAIKENFKGEIERYYGSAASSVCIRNPEACLNHLKSLIGVPIDDVSTIEYKKYHSGAELVPSKNNRNTVAFNLGSSVYPVEEILAMSLDDIKSRAEDHLKHAVPGSYSVINDAVITVPTFFTQSQRLALKDAAEISGLKVVGLVDDGISVAVNYASSRQFDGNKQYHMIYDMGAGSLQATLVSISSNEDGGIFIDVEAIAYDNSLGGQLFTQSVYDILLQKFLSEHPSFSESDFNKNSKSMSKLWQSAEKAKTILSANTDTRVSVESLYNDIDFRTTITRDEFEDYNAEHVHRITAPIIEALSHPLNENLTSPFPLTSLSSVILTGGSTRVPMVKKHLESLLGSELIAKNVNADESAVFGSTLRGVTLSQMFKARQMTVNERSVYDYCVKVGSSEINVFPVGTPLDTKKVVELENVDNGNQLTVGLYENGHLFANQEVSDLKKSIKSLTQEGKECSNIIYEATFELSESRLFSLTRLQAKCADKVESLPPVDTESDDAKSENSTSSENTEKSNKKLFYPVTIPTQLKFVHVKPMGSSTKISSSLKIKELNKKDAVKRSIEELKNQLESKLYRVRSYLEDEQVVQKGPASQVEALSTQVAENLEWLDYDSDDASAKDIRDKLNFVSESVSFIKNYIDLSDVTLDNNLFTMIYNTTSNSMQNVQELMLNMSEDALSLMQQYEKEGLDFAKESQKIKIKSPPLSDKELDGLFNVVTEKLEYVRTLTEEDGIVGLPREELFKLYQELQNYSSRFEEIMTSLKDVHSQRINRLTDKLNKHIERVNNEALKAALKEAKRQQEEEKSHEQNDEEEQGSSSTSHTKAETEE

SEQ ID NO: 39 - protein, 903 aa, Candida boidinii
MKLFNQIICILAIISPILASILGIDFGQQFTKSALLGPGVNFEILLTVDSKRKDISGLAMAIAPNSNNEIQRSFGSSSLSTCVKNPQACFTSFKSLLGKAIDDESTTQLYLKSHPGIELAPANYSRNTIDFKYNHDSYPVEEILAMYFRDIKSRADDYLGDHASPGYTKVQKTAITVPGFFNQAQRRAILDAAEIAGLDVVSLVDDGIAIAAEYASSRAFEIEKEYHLIYDMGAGSTKATLVSFSQNNSDISIVNEGYGFDETLGGELLTNSIKELLISKFLAANPKVKISDFLSNSRAITRLLQSAEKAKSVLSANTETRVSIENIYNEIDFKTTITRAEYEEINSPIMERITAPILKAIQSNSERRDSEDEDQPEITLKDIKSVILAGGSTRVPFVQRHLISLVGEDVISKNVNADEAAVLGTTLRGVQISGLFRSKRMTVVESTTNDFCYKIVSNELDEKDSNLVTVFPVNAKINSKKSVKLNQLKDTFSDFELDFYSNGEFISQANISPSEKFDNKLCTNGTSYIARLELDNSGLASLTSVDQFCYFEKITKLANNSTETDETDKTSSKTSEEEAATTSIASKKEKLEPKIKYPYIRPMGVSTKKICKNRISKLDTKDAVRIEKATTVNKLEAILYSLRSHLDEDEIAEFVNSKSTFIDDISTFVKENLEWLEETYQLPDLEVIQSKLEAATKKVSDIKEFTRVHKSLRDSEFYKNMTTISNEAMFGIQDFLLTMSEDLTSIHTNYTMAGVDINEANKKIEVMTNPFDEATIKEHFDALGELLDKIKTLTEDEDVLAEKSIDYLFQLFKDVVKELEVLTKIKNVLVRIHTKRITKLQEYLVKQLKKKLKAERKSKSKASSKSAKSEEEVTTTSIAPENTDSSNASDSSSDSSTVQKDEL

SEQ ID NO: 40 - protein, 798 aa, Ogataea polymorpha
MKVLGLVALIFIIVQGWASLLAIDFGQDYSKAALVAPGVAFDLVLTDEAKRKHQSGVAISAKDGEIERKFNSHALSACTRSPQSCFFELKSLIGRQIDEPQVTRFEKKYRGVKIVPASSQRRTVAFDVDGQVYLLEEVLGMVLEEIKKRAELHWDQTLGGGSSNTISDVVLSVPDFLDQAQRTALVDAAEIAGLNVVALIDDGIAVALNYASTRDFEQKQYHVIYDVGAGSTKATLVSFSKDNETLRVENEGYGYDETFGGNLFTESLQAIIEDKFLAQTKIKPETLWSDARAMNRLWQSAEKAKLVLSANSETKVSVESLINDIDLKVVVSRDEFEEYMTEHMDRIVAPLAAAMGDRKVESVILAGGSTRVPFVQKHLVKYLGGDELLSKNVNADEAAVFGTLLGGISVSGKFRTRPIELVQHASRNFELAAGGHMTVVFNETTASREAVVALPGLKDTFGEVQVDLFEAGQLFAQYKFKNELNSTVCPNGVEYLANCTLDPRKLFLLHSLEAVCAGDGAVRSSLTAKPLHPGYKPLGSLAKYQSASKLRSLTNQDKQRQQRDALINSLEASLYDLRSYTEDENVVANGPSSMVRAAREMVSELLEWLEDVPAKATVKDIQEKYDDVRVMRIKLETLVNHGDRLLSLAEFTRLKEKALETMYKLQDFMVVMSQDALSLKANFTELGLDFEEANRRVKVKVPEVDEQELEQRMKRISDFVGVVDHFETHKDEIETKDRETLFELRETVLEELKQVQSTYRALKQAHEKRVRGLKEQLKKADKKADKTQEAEPSGHDEL

SEQ ID NO: 41 - protein, 881 aa, Saccharomyces cerevisiae
MRNVLRLLFLTAFVAIGSLAAVLGVDYGQQNIKAIVVSPQAPLELVLTPEAKRKEISGLSIKRLPGYGKDDPNGIERIYGSAVGSLATRFPQNTLLHLKPLLGKSLEDETTVTLYSKQHPGLEMVSTNRSTIAFLVDNVEYPLEELVAMNVQEIANRANSLLKDRDARTEDFVNKMSFTIPDFFDQHQRKALLDASSITTGIEETYLVSEGMSVAVNFVLKQRQFPPGEQQHYIVYDMGSGSIKASMFSILQPEDTTQPVTIEFEGYGYNPHLGGAKFTMDIGSLIENKFLETHPAIRTDELHANPKALAKINQAAEKAKLILSANSEASINIESLINDIDFRTSITRQEFEEFIADSLLDIVKPINDAVTKQFGGYGTNLPEINGVILAGGSSRIPIVQDQLIKLVSEEKVLRNVNADESAVNGVVMRGIKLSNSFKTKPLNVVDRSVNTYSFKLSNESELYDVFTRGSAYPNKTSILTNTTDSIPNNFTIDLFENGKLFETITVNSGAIKNSYSSDKCSSGVAYNITFDLSSDRLFSIQEVNCICQSENDIGNSKQIKNKGSRLAFTSEDVEIKRLSPSERSRLHEHIKLLDKQDKERFQFQENLNVLESNLYDARNLLMDDEVMQNGPKSQVEELSEMVKVYLDWLEDASFDTDPEDIVSRIREIGILKKKIELYMDSAKEPLNSQQFKGMLEEGHKLLQAIETHKNTVEEFLSQFETEFADTIDNVREEFKKIKQPAYVSKALSTWEETLTSFKNSISEIEKFLAKNLFGEDLREHLFEIKLQFDMYRTKLEEKLRLIKSGDESRLNEIKKLHLRNFRLQKRKEEKLKRKLEQEKSRNNNETESTVINSADDKTTIVNDKTTESNPSSEEDILHDEL

SEQ ID NO: 42 - protein, 863 aa, Kluyveromyces lactis
MRIVFWFLLAIQSLTTCFAAVVGLDFGTHYVKEMVVSLKAPLEIVLNPESKRKDASALAIRSWDSQNYLERFYGSSAVALATRFPSTTFMHLKSLLGKHYEDNLFYYHREHPGLEFVNDASRNAIAFEIDTNTTLSVEELVSMNLKQYMERANQLLKESDDSDNVKSVAIAIPEYFSQEQRAALLDATYLAGIGQTYLCNDAIAVAIDYASKQKSFPAGKPNYHVIYDMGAGSTTASLISILQPENITLPLRIEFLGYGHTESLSGSVLSLAIVDLLENDFLESNPNIRTEQFESDASAKAKLVQAAEKAKLVLSANSDASISIESLYHDLDFKTTITRAKFEEFVAELQSVVIEPILSTLESPLNGKALNVKDLDSVILTGGSTRVPFVKKQLENHLGASLISKNVNSDESAVNGAAIRGVQLSKEFKTRPMKVIDRTTHSFGFSIQNTNISKLVFDAGSEYPKEINLQLPGMELKDTVLKIDLTEDERVFKTIFADVDSKLQSSSLSNCSTAVTYNVTLSLNTDQVFDVQSVVASCLTHEEVPTGTEKEHKRTVSEHIQKHPIPHTVEFTCVKPLSNTEKKERFNKLHKWDQKDKLLLERQRLLNDLEASLYAARELVEDAKELETPPTSYIQQLENMITQYLEFVDDPSSLRTKNIKTMKSNLAELQQRLEIYMDRDNKQLDVEGFRALFDKGEKYLELLSKIQQKSLSELSPLNKNFESLGLNVSEEYTKVKPPKSKTVPFEILNGTIDLLHSQLKHIRDIIEDNNSTYAIEDLFEQKLEVDSLYEKIELLVKKIRAEHKYRLKLLQSVYDRRLTAQKREQEIAKEAQQADGENNDSIKTMEEESIEEHEDANFEQDEL

SEQ ID NO: 43 - protein, 1007 aa, Yarrowia lipolytica
MKVAHIIQLAAMVATALAAVLAIDYGQEYTKAALLSPGINFEIVLTQDSKRKQPSAIGFKGKADSKFGLERVYGSPAVLMEPRFPSDVVLYHKRLLGGRPKLDNPNYKEYTQMRPACMAVPSNSSRSAIAFQVKDSEWSAEELLAMQISDIKSRADDMLKTQSKSNTDTVKDVVMTVPPHFTHSQRLALADAVDLAGLKLIALVSDGTATAVNYVSTRKFTDEKEYHVVYDMGAGSASATLFSVQDVNGTPVIDIEGVGYDEALAGQDMTNMMVKILAASFMEQNKDKVQLQTFIRDVKAAAKLWKEAERAKAILSANQEVSVSIEAVHNGIDFKTTVTRDDYVRSIEKISTRLNGPLEKALAGFADSPVALKDVKSVILTGGVTRTPVIQEKLKELLGDVPISKNVNTDESIVLGSLLRGVGISSIFKSRDIKVIDRTPHEFDLRLDVLGAKDEILRSEKANVFSKGAAQGESVVSKLDISEIGNANLYLLEDGDSFVRLDVRDMDAIKKELNCEKSAELHVPFDLTLSGTIKVGKAKVVCKGGDAEADAEVTVDDPVEDVVVEEEVVEGETVEGDAKAAKDSKDSKDSKKASKKVDTSRYVPHKTRFVGTKPLTSAAKLKISGHLRSLARKDAERLATSDAANKLESTIYHIKHLIEDAVDQDKVADIKKKIEDAAAWFEEDGLTAGIQELTEKLSVVQPLEDFFKTAGEAIADKATAAASAAGEFVDQAAAAAGVKAGEAADAAKGAADAAGKKAKKAKKAAGKAASQAEEDVLDQLKDANDLIKNIAQLARESGNDVPSEEDIEREMKRAAEGGDSSDSADLSGHLETLMGLQDMLNELNGGEAPSAPGLDVTAIAGITRTIQRLSDKLTELGTPPKDEDDMFRMLGIDPQTFHKFSEEAFEDQASPADQLMDSIGFLQQVLAQDESPDPAALEKMRANIAERQERIAKVAEVAERNQKRQIAALENMLKNAEKTIDISIYNLKQQAPKTASVEDKKAEHDEL

SEQ ID NO: 44 - protein, 1000 aa, Aspergillus niger
MAPGSQRRPYASLTSLPVLSLILPFLLFVLSFPAPAAAAGSAVLGIDVGTEYLKATLVKPGIPLEIVLTKDSKRKESAAVAFKPTREADASFPERFYGGDALALAARYPDDVYSNLKTLLGLPFDADNELIKSFHSRYPALRLEEAPGDRGTVGLRSNRLGEAERKDAFLIEEILAMQLKQIKANADTLAGKGSDITDAVITYPSFYTAAEKRSLELAAELAGLNVDAFISDNLAVGLNYATSRTFPSVSDGQRPEYHIVYDMGAGSTTASVLRFQSRSVKDVGRFNKTVQEVQVLGTGWDKTLGGDALNDLIVQDMIASLVEEKKLKDRVSPADVQAHGKTMARLWKDAEKARQVLSANTETGASFESLYEEDLNFKYRVTRAKFEELAEQHIARVGKPLEQALEAAGLQLSDIDSVILHGGAIRTPFVQKELERVCGSANKIRTSVNADEAAVFGAAFKGAALSPSFRVKDIRASDASSYAVVLKWDSESKERKQKLFTPTSQVGPEKQVTVKNLDDFEFSFYHQIPVDGNVVESPILGVKTQNLTASVAKLKEDFGCTAANITTKFAIRLSPVDGLPEVASGTVSCEVESAKKGSVVEGVKGFFGLGNKDEQVPLGEEGEPSESITLEPEEPQAATTSSADDATSTTSAKESKKSTPATKLESISISFTSSPLGIPAPTEAELARIKSRLAAFDASDRERALREEALNELESFIYRSRDLVDDEEFAKVVKPEQLTTLQERASEASDWLYGDGDDAKTADFRAKLKSLREIVDPALKRKKENAERPARVELLQQVLKNAKSVIDVMEQQIQQDEDLYSSVTASSSSSSTATESSTSSSTTTGSSSSVDLDEDPYATTSTSSTTKTASATTTPKPSGPKYSIFQPYDLTSLSKTYESTNTWFETQLALQEQLTMTDDPALPVAELDTRLKELERVLNRIYDKMGAAAAKSGKEQSKKNNNNNGKSSKKEKAKAQEEQKKPAKEEEQKDDKKANRKDEL

SEQ ID NO: 45 - protein, 985 aa, Trichoderma reesei
RKSPLLKLLGAAFLFSTNVLAISAVLGVDLGTEYIKAALVKPGIPLEIVLTKDSRRKETSAVAFKPAKGALPEGQYPERSYGADAMALAARFPGEVYPNLKPLLGLPVGDAIVQEYAARHPALKLQAHPTRGTAAFKTETLSPEEEAWMVEELLAMELQSIQKNAEVTAGGDSSIRSIVLTVPPFYTIEEKRALQMAAELAGFKVLSLVSDGLAVGLNYATSRQFPNINEGAKPEYHLVFDMGAGSTTATVMRFQSRTVKDVGKFNKTVQEIQVLGSGWDRTLGGDSLNSLIIDDMIAQFVESKGAQKISATAEQVQSHGRAVAKLSKEAERLRHVLSANQNTQASFEGLYEDVDFKYKISRADFETMAKAHVERVNAAIKDALKAANLEIGDLTSVILHGGATRTPFVREAIEKALGSGDKIRTNVNSDEAAVFGAAFRAAELSPSFRVKEIRISEGANYAAGITWKAANGKVHRQRLWTAPSPLGGPAKEITFTEQEDFTGLFYQQVDTEDKPVKSFSTKNLTASVAALKEKYPTCADTGVQFKAAAKLRTENGEVAIVKAFVECEAEVVEKEGFVDGVKNLFGFGKKDQKPLAEGGDKDSADASADSEAETEEASSATKSSSSTSTTKSGDAAESTEAAKEVKKKQLVSIPVEVTLEKAGIPQLTKAEWTKAKDRLKAFAASDKARLQREEALNQLEAFTYKVRDLVDNEAFISASTEAERQTLSEKASEASDWLYEEGDSATKDDFVAKLKALQDLVAPIQNRLDEAEKRPGLISDLRNILNTTNVFIDTVRGQIAAYDEWKSTASAKSAESATSSAAAEATTNDFEGLEDEDDSPKEAEEKPVPEKVVPPLHNSEEIDTLEVLYKETLEWLNKLERQQADVPLTEEPVLVVSELVARRDALDKASLDLALKSYTQYQKNKPKKPTKSKKAKKQDKTKSADKAGPTFEFPEGSVPLSGEELEELVKKYMKEEEETRRQAEG

SEQ ID NO: 46 - protein, 848 aa, Schizosaccharomyces pombe
MKRSVLTIILFFSCQFWHAFASSVLAIDYGTEWTKAALIKPGIPLEIVLTKDTRRKEQSAVAFKGNERIFGVDASNLATRFPAHSIRNVKELLDTAGLESVLVQKYQSSYPAIQLVENEETTSGISFVISDEENYSLEEIIAMTMEHYISLAEEMAHEKITDLVLTVPPHFNELQRSILLEAARILNKHVLALIDDNVAVAIEYSLSRSFSTDPTYNIIYDSGSGSTSATVISFDTVEGSSLGKKQNITRIRALASGFTLKLSGNEINRKLIGFMKNSFYQKHGIDLSHNHRALARLEKEALRVKHILSANSEAIASIEELADGIDFRLKITRSVLESLCKDMEDAAVEPINKALKKANLTFSEINSIILFGGASRIPFIQSTLADYVSSDKISKNVNADEASVKGAAFYGASLTKSFRVKPLIVQDIINYPYLLSLGTSEYIVALPDSTPYGMQHNVTIHNVSTIGKHPSFPLSNNGELIGEFTLSNITDVEKVCACSNKNIQISFSSDRTKGILVPLSAIMTCEHGELSSKHKLGDRVKSLFGSHDESGLRNNESYPIGFTYKKYGEMSDNALRLASAKLERRLQIDKSKAAHDNALNELETLLYRAQAMVDDDEFLEFANPEETKILKNDSVESYDWLIEYGSQSPTSEVTDRYKKLDDTLKSISFRFDQAKQFNTSLENFKNALERAESLLTNFDVPDYPLNVYDEKDVKRVNSLRGTSYKKLGNQYYNDTQWLKDNLDSHLSHTLSEDPLIKVEELEEKAKRLQELTYEYLRRSLQQPKLKAKKGASSSSTAESKVEDETFTNDIEPTTALNSTSTQETEKSRASVTQRPSSLQQEIDDSDEL

SEQ ID NO: 47 - protein, 372 aa, Komagataella phaffii
MKVTLSVLAIASQLVRIVCSEGENICIGDQCYPKNFEPDKEWKPVQEGQIIPPGSHVRMDFNTHQREAKLVEENEDIDPSSLGVAVVDSTGSFADDQSLEKIEGLSMEQLDEKLEELIELSHDYEYGSDIILSDQYIFGVAGLVPTKTKFTSELKEKALRIVGSCLRNNADAVEKLLGTVPNTITIQFMSNLVGKVNSTGENVDSVEQKRILSIIGAVIPFKIGKVLFEACSGTQKLLLSLDKLESSVQLRGYQMLDDFIHHPEEELLSSLTAKERLVKHIELIQSFFASGKHSLDIAINRELFTRLIALRTNLESANPNLCKPSTDFLNWLIDEIEATKDTDPHFSKELKHLRFELFGNPLASRKGFSDEL

SEQ ID NO: 48 - protein, 379 aa, Komagataella pastoris
MPKTLSSMKVSLSVLAIATQLVRIVCSEEENICIGDQCYPKNFEPDKEWKPVQEGQIIPPGSHVRMDFNTHQREAKLVDENDDIDSSLMGVAVVDATDTFADDHSLEKIIGLSVSQLDEKLEELVELSHDYEYGSDIILNDQYIIGVAGLVPTKTQFASELKEKALRIVGSCLRNNADAVEKLLGTVPNTITIEFISNLVGKVNTTEENVDPVEQKRILSIIGAIIPFNIGKVLFEACFGTQKLLLSLDKLDDSVQLKAYQVLDDFIHHPQEELLSSLTEKERLVKHIELIQSFFASGKHSLHEAINRELFSRLVALRSDLESTSTNLCTPSTDFLNWLIDEIEATKEVNPHFSQELKHLRFEFFGNPLASRKGFSDEL

SEQ ID NO: 49 - protein, 356 aa, Ogataea parapolymorpha
MLCLLLFGGVSLAKLICPDPNPLNCYPELFEPSTDWKPVKEGQIIPGGLDIRLNIDTLEREAKLTGNSQPNENGAVIVPEDIMELDEEQNLSEALRYLSKFVDHGVGDSATLLRKLEFISEMSSDSDYGVDTMQYIQPLIRLSGLYGEEGLKQIDDENRDEIRELATIILASSLRNNPEAQRKFLQYFSDPMDFVDHLTAKIQNDVLLRRRLGILGSLLNSGSLIDGFESIKKKLLILYPQLENQATKQRLMHIISDITGDVEDEDMDRQFANIAQDTLIDQKALDDGTLTLLDELKKLKLNNRNLFKAKSEFLEWLNVRMEALKAAKDPKLEEFRSLRHEIFGNPKAMRKSYDEL

SEQ ID NO: 50 - protein, 421 aa, Saccharomyces cerevisiae
MVRILPIILSALSSKLVASTILHSSIHSVPSGGEIISAEDLKELEISGNSICVDNRCYPKIFEPRHDWQPILPGQELPGGLDIRINMDTGLKEAKLNDEKNVGDNGSHELIVSSEDMKASPGDYEFSSDFKEMRNIIDSNPTLSSQDIARLEDSFDRIMEFAHDYKHGYKIITHEFALLANLSLNENLPLTLRELSTRVITSCLRNNPPVVEFINESFPNFKSKIMAALSNLNDSNHRSSNILIKRYLSILNELPVTSEDLPIYSTVVLQNVYERNNKDKQLQIKVLELISKILKADMYENDDTNLILFKRNAENWSSNLQEWANEFQEMVQNKSIDELHTRTFFDTLYNLKKIFKSDITINKGFLNWLAQQCKARQSNLDNGLQERDTEQDSFDKKLIDSRHLIFGNPMAHRIKNFRDEL

SEQ ID NO: 51 - protein, 490 aa, Kluyveromyces lactis
MRVKCVNRAIYVLTVLLFSRLVVSQVVLTPSNSNADPKQKDTANTVAAVEANNDANIAKKDAESDLVIGDHLVCNTKECYPIGFVPSTEWKEIRPGQRLPPGLDIRVSLEKGVREAKLPEPGSENIGNEEEDVKGLVLGAEGSTLSESELKETSEDLENEQSGFKLNNAEKESDILQQETDLKIAVSDNAEATSNEPAGHEFSEDFAKIKSLMQSPDEKTWEEVETLLDDLVEFAHDYKKGFKILSNEFELLEYLSFNDTLSIQIRELAARIIVSSLRNNPPSIDFVNEKYPQTTFKLCEHLSELQASQGSKLLIKRFLSILDVLLSRTEYVSIKDDVLWRLYQIEDPSSKIKILEIIAKFYNEKNEQVIDTVQQDMKTVQKWVNELTTIIQTPELDELHLRSFFHCISFIKTRFKNRVKIDSDFLNWLIDEIEVRNEKSKDDIYKRDVDQLEFDNQLAKSRHAVFGNPNAARLKERLFDDDDTLIADEL

SEQ ID NO: 52 - protein, 505 aa, Candida boidinii
MKFEFSLLVLIFSKLLVAANTAGGDMVCPDDNPDNCYPKIFVPTNEWQEIKPEQHIPAGLHVRMNIENMGREAKLPEKSSNSQINKDIQAVAVDLGGDAADNGGDVNNAVVAVGEVHDAEENIKVENGNGQGNKKSNGSRGKPAPGELLNALKGVEEFLNNDRTDNVEGLMGYLEILDDLSHDIDYGVDISKNPMSLIQLTGIYTFEQPDIYETKLKGKTTDSLKIQDMSMRVLSSTIRNNDEALDNIVELFNGSKDKLYKVIMEKLEKLNNNSFENIIQRRRLGLLNSILGHEEIASSFCCLSNDLTLLHLYSKITDKESKAKIINILHDLRIAPDYCHSENIVNLSPQDIQDSLQLKKRYQDDNLNISESVIVDEEDEEAFGDITDVDLKYSIVAQRMLRKYGLISNYKAREILQDLIDLKNNKKNSLKISSRFLNWMEYQIDQVKQLNNNLSGSNNQDDDNQQRFTIESRDGERDYLDYLIVARHEVFGNSHAGRKASADEL

SEQ ID NO: 53 - protein, 426 aa, Yarrowia lipolytica
MKFSKTLLLALVAGALAKGEDEICRVEKNSGKEICYPKVFVPTEEWQVVWPDQVIPAGLHVRMDYENGVKEAKINDPNEEVEGVAVAVGEEVPEGEVVIEDLTEENGDEGISANEKVQRAIEKAIKEKRIKEGHKPNPNIPESDHQTFSDAVAALRDYKVNGQAAMLPIALSQLEELSHEIDFGIALSDVDPLNALLQILEDAKVDVESKIMAARTIGASLRNNPHALDKVINSKVDLVKSLLDDLAQSSKEKADKLSSSLVYALSAVLKTPETVTRFVDLHGGDTLRQLYETGSDDVKGRVSTLIEDVLATPDLHNDFSSITGAVKKRSANWWEDELKEWSGVFQRSLPSKLSSKVKSKVYTSLAAIRRNFRESVDVSEEFLEWLDHPKKAAAEIGDDLVKLIKQDRGELWGNAKARKYDARDEL

SEQ ID NO: 54 - protein, 406 aa, Trichoderma reesei
MRPLALIFALILGLLLCLAAPATASSSSSQHSPQAASDESDLICHTSNPDECYPRVFVPTHEFQPVHDDQQLPNGLHVRLNIWTGQKEAKINVPDEANPDLDGLPVDQAVVLVDQEQPEIIQIPKGAPKYDNVGKIKEPAQEGDAQTEAIAFAETFNMLKTGKSPSAEEFDNGLEGLEELSHDIYYGLKITEDADVVKALFCLMGARDGDASEGATPRDQQAAAILAGALSNNPSALAEIAKIWPELLDSSCPRDGATISDRFYQDTVSVADSPAKVKAAVSAINGLIKDGAIRKQFLENSGMKQLLSVLCQEKPEWAGAQRKVAQLVLDTFLDEDMGAQLGQWPRGKASNNGVCAAPETALDDGCWDYHADRMVKLHGTPWSKELKQRLGDARKANSKLPDHGEL

SEQ ID NO: 55 - protein, 312 aa, synthetic construct
  REGION 1..312: note = Fab HC
MRFPSIFTAVLFAASSALAAPVNTTTEDETAQIPAEAVIGYSDLEGDFDVAVLPFSNSTNNGLLFINTTIASIAAKEEGVSLEKREVQLVQSGGGLVQPGGSLRLSCAASGFTFSHYWMSWVRQAPGKGLEWVANIEQDGSEKYYVDSVKGRFTISRDNAKNSLYLQMNSLRAEDTAVYFCARDLEGLHGDGYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 56 - protein, 299 aa, synthetic construct
  REGION 1..299: note = Fab LC
MRFPSIFTAVLFAASSALAAPVNTTTEDETAQIPAEAVIGYSDLEGDFDVAVLPFSNSTNNGLLFINTTIASIAAKEEGVSLEKRAIQLTQSPSSLSASVGDRVILTCRASQGVSSALAWYQQKPGKAPKLLIYDASSLESGVPSRFSGSGSGPDFTLTISSLQPEDFATYFCQQFNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 341 aa, synthetic construct
  REGION 1..341: note = scFv
MRFPSIFTAVLFAASSALAAPVNTTTEDETAQIPAEAVIGYSDLEGDFDVAVLPFSNSTNNGLLFINTTIASIAAKEEGVSLEKRQEQLMESGGGLVTLGGSLKLSCKASGIDFSHYGISWVRQAPGKGLEWIAYIYPNYGSVDYASWVNGRFTISLDNAQNTVFLQMISLTAADTATYFCARDRGYYSGSRGTRLDLWGQGTLVTISSGGGGSGGGGSGGGGSELVMTQTPPSLSASVGETVRIRCLASEFLFNGVSWYQQKPGKPPKFLISGASNLESGVPPRFSGSGSGTDYTLTIGGVQAEDVATYYCLGGYSGSSGLTFGAGTNVEIKGGHHHHHH

SEQ ID NO: 58 - protein, 299 aa, Komagataella phaffii
MKLHLVILCLITAVYCFSAVDREIFQLNHELRQEYGDNFNFYEWLKLPKGPSSTFEDIDNAYKKLSRKLHPDKIRQKKLSQEQFEQLKKKATERYQQLSAVGSILRSESKERYDYFVKHGFPVYKGNDYTYAKFRPSVLLTIFILFALATLTHFVFIRLSAVQSRKRLSSLIEENKQLAWPQGVQDVTQVKDVKVYNEHLRKWFLVCFDGSVHYVENDKTFHVDPEEVELPSWQDTLPGKLIVKLIPQLARKPRSPKEIKKENLDDKTRKTKKPTGDSKTLPNGKTIYKATKSGGRRRK

SEQ ID NO: 59 - protein, 299 aa, Komagataella pastoris
MKLHLVILCLITAVYCFSAVDREIFQLNHELRQEFGDNFNFYEWLKLPKGPSSTFEDIDNAYKKLSRKLHPDKVRQKKLSQQQFQQLKKKATERYQQLSAVGSILRSESKERYDYFLKHGFPVYKGNDYTYAKFRPSVLITVFILFALATLTHFVFIRLSAVQSRKRLSSLIEENKQLAWPQGVQDVTKVKDVKVYNEHLRKWFLVCFDGSVHYVENDKTYHVDPEEVELPSWQDSLPGKVIVRLIPQLAKKPRPPKETKKEDLDEKSKKTKKPTGDSKTLPNGKTIYKATKSGGRRRK

SEQ ID NO: 60 - protein, 323 aa, Ogataea parapolymorpha
MRLLFWLAIFSATVFAAWSAEDLEIFKLQHELVKDTKKETNFYEYLGLSNGPKASYDEINKAYKKMSRKLHPDKVRRKEGMSQKAFERRKKAAEQRFQRLSLIGTILRGERKERYDYYLKHGFPAYTGTGFALSKFRPGPVLALVVVVVLFSAVHYIMLKLNTQQKRKRVESLINDLKAKAFGPSMLPGTNFSDQRVAHMDKLFVVKFDGSVWLVDKELKEGEDYIVDEDGRQIFRVEAEPKNRKQRRAKKDKDEVLLPVTPDDVEEVTWRDTLVVRFVLWAISKLEKKPKTHDKADKGTIRRLPNGKVKKVRPTGENGEKNK

SEQ ID NO: 61 - protein, 404 aa, Candida boidinii
MRSFKIIFFVLAFFTAIALCWTHEDIEIFEINESLKKETKDPEMNFYKYLNLPSGPKSSYDQISRAFKKLSRKYHPDKYKPDFNNDEKTINKQKKNWEKRFQNIGAIAEILRSENKDRYDFFYKNGFPTINDENEYVYNKYRPSFLITLAVIFVIISVLHFIVIKSNNTQQRQRIESLINEIKTRAFGNGTPTDFKDRKVYHDGLDKYFVAKFDGSVYLLDESHLSSGTPIEELSPEEIDKIEMQRHGYNGPKLAKGVFYYKDDTYKNRRTRRSELKHGSDEDEDVLLQMSVDEVPLVTLKDMLFIRFLSSIYNTTLERLIPKSQPETETSGSKKKTIPTTKSKDSTTEEDFEILNLEDANPDSNETSKSSKEANTVLGSKTKKTSSGEKKVLPNGQVIYSRKK

SEQ ID NO: 62 - protein, 277 aa, Kluyveromyces lactis
MLSSSRPVTYALFLSLFAAVAYCFTRDEIEIFQLQQELHTKYGSNMDFYQFLKLPKLKQSTSAEITKNFKKLAKKYHPDKNPKYRKLYERINLITKLLSDEGHRKTYDYYLKNGFPKYDYKKGGFFFNRVTPSVWFTFFFLYVLAGVIHLVLLKLHNNANKKRIENFVAKVREQDTTNSLGESKLVFKESEDSEDKQLLVRFGEVFVIQPDESLAKISTDDIIDPGINDTLLVKLPKWIWNKTLGKFINIGTSKSQQPNKGSPNKNKRNSKINSKAQ

SEQ ID NO: 63 - protein, 397 aa, Aspergillus niger
MKSIALRLFVFVALIVLAAAWTKEDYEIFRLNDELAAAEGPNVTFYDFLGAKPNANQDELSKAYRQKSRLLHPDKVKRSFIANSSKDKSRSKSSKSGVHVNQGPSKREIAAAVKEAHERSARLNTVANILRGPGRERYDHFLKNGFPKWKGTGYYYSRFRPGLGSVLIGLFLVFGGGAHYAALVLGWKRQREFVDRYIRQARRAAWGDESGVRGIPGLDGASAPAPTPAPAPEPEQSAMPMNRRQKRMMDRENRKEGKKGGRAASRNSGTATPTSEPQMEPSGERKKVIAENGKVLIVDSLGNVFLEEETEDGERQEFLLDVDEIQRPTIRDTLVFRLPGWVYSKTVGRLLGSSNAVNSGAESEEEPSEIVEESTEGAASSARSSKARRRGKRSQRS

SEQ ID NO: 64 - protein, 414 aa, Trichoderma reesei
MKIEYLVVGVLSLLTPLAAAWSKEDREIFRIRDEIAAHESDPAASFYDILGVTPSASQDDINKAYRKKSRSLHPDKVKQQLRAEKAQADKKKGAGGGSAASSSKGPTQAEIRKAVKEASERQARLSLIANILRGPARDRYDHFLANGFPLWKGTDYYYNRYRPGLGTVLVGVFMMGGGAIHYLALYMSWKRQREFVERYVTFARNAAWGNDAGIPGVDAMPAPAPAPAPEEDEAAAPAQPRNRRERRMQEKETRKDDGKSSKKARKAVTSKSSSSAPTPTGARKRVVAENGKILVVDSQGDVFLEEEDEEGNVNEFLLDPNELLQPTFKDTAVVRVPVWVFRSTVGRFLPKGAAQAEAEETHEEDSDAAQNTPPSSESAGDDFEILDKSTDSLSKVKTSGAQQGKATKRKTTKK

SEQ ID NO: 65 - protein, 295 aa, Saccharomyces cerevisiae
MNGYWKPALVVLGLVSLSYAFTTIETEIFQLQNEISTKYGPDMNFYKFLKLPKLQNSSTKEITKNLRKLSKKYHPDKNPKYRKLYERLNLATQILSNSSNRKIYDYYLQNGFPNYDFHKGGFYFSRMKPKTWFLLAFIWIVVNIGQYIISIIQYRSQRSRIENFISQCKQQDDTNGLGVKQLTFKQHEKDEGKSLVVRFSDVYVVEPDGSETLISPDTLDKPSVKNCLFWRIPASVWNMTFGKSVGSAGKEEIITDSKKYDGNQTKKGNKVKKGSAKKGQKKMELPNGKVIYSRK

SEQ ID NO: 66 - protein, 287 aa, Yarrowia lipolytica
MKFSIIFLVTLFALVFAQGGNQWSKEDREIFDLNLAVQKDLNPDNSKPVSFYQWLDTERKASVDEVTKSYRKLSRQLHPDKNRKVPGATDRFTRLGLVYKILINKDLRKRYDFYLKNGFPREGENGEFVFKRFKPGVGFALFVLYFLIGLGSYVVKYLNAKKIKSTIERVEREVRKEASRKNGVRLPATTDVIVDGRQYCYYNTGEIHLVDTDNNIEHPISSQGVEMPGIKDSLWVTLPVALFNLVKPKSAAEKAEEAKIQQEKEAKEERERPKPKAATKVGGRRRK

SEQ ID NO: 67 - protein, 303 aa, Schizosaccharomyces pombe
MSRIFILLLLFGVCLAWTSSDLEIFRVVDSLKSILKNKATFYELLEVPTKASIKEINRAYRKKSILYHPDKNPKSKELYTLLGLIVNILRNTETRKRYDYFLKNGFPRWKGTGYLYSRYRPGLGAVLVLLFLLISIAHFVMLVISSKRQKKIMQDHIDIARQHESYATSARGSKRIVQVPGGRRIYTVDSITGQVCILDPSSNIEYLVSPDSVASVKISDTFFYRLPRFIVWNAFGRWFARAPASSEDTDSDGQMEDEEKSDSVHKSSFSSPSKKEASIKAGKRRMKRRANRIPLSKNTNREN

SEQ ID NO: 68 - DNA, 288 bp, Pichia pastoris
  source 1..288: mol_type = genomic DNA
gacaggtctcccatgtctacagcaattccaggaggacagagaacgttagctaaaagaagagcagcaaacttggataagaaacaggatgaaccaacctccgccagatctgccggtgctggaggttcttcgtctaccatgctaaagttgtacacagacgaggcccaaggtttgaaagttgatcctttaattgttcttgttcttgctgttggtttcattttcagtgtcattggtttgcacgttgttgctaagctgacaggaaagttgatcaactaagcttggagaccgatc

SEQ ID NO: 69 - DNA, 34 bp, synthetic construct
  misc_feature 1..34: note = oligonucleotide primer PP7435_Chr2-0220
  source 1..34: mol_type = other DNA
gacaggtctcccatgtctacagcaattccaggag

SEQ ID NO: 70 - DNA, 39 bp, synthetic construct
  misc_feature 1..39: note = oligonucleotide primer PP7435_Chr2-0220 reverse
  source 1..39: mol_type = other DNA
gatcggtctccaagcttagttgatcaactttcctgtcag

SEQ ID NO: 71 - DNA, 2068 bp, Pichia pastoris
  source 1..2068: mol_type = genomic DNA
gatctaggtctcccatgctgtcgttaaaaccatcttggctgactttggcggcattaatgtatgccatgctattggtcgtagtgccatttgctaaacctgttagagctgacgatgtcgaatcttatggaacagtgattggtatcgatttgggtaccacgtactcttgtgtcggtgtgatgaagtcgggtcgtgtagaaattcttgctaatgaccaaggtaacagaatcactccttcctacgttagtttcactgaagatgagagactggttggtgatgctgctaagaacttagctgcttctaacccaaaaaacaccatctttgatattaagagattgatcggtatgaagtatgatgccccagaggtccaaagagacttgaagcgtctgccttacactgtcaagagcaagaacggccaacctgtcgtttctgtcgagtacaagggtgaggagaagtctttcactcctgaggagatttccgccatggtcttgggtaagatgaagttgatcgctgaggactacttaggaaagaaagtcactcatgctgtcgttaccgttccagcctacttcaacgacgctcaacgtcaagccactaaggatgccggtctaatcgccggtttgactgttctgagaattgtgaacgagcctaccgccgctgcccttgcttacggtttggacaagactggtgaggaaagacagatcatcgtctacgacttgggtggaggaaccttcgatgtttctctgctttctattgagggtggtgctttcgaggttcttgctaccgccggtgacacccacttgggtggtgaggactttgactacagagttgttcgccacttcgttaagattttcaagaagaagcataacattgacatcagcaacaatgataaggctttaggtaagctgaagagagaggtcgaaaaggccaagcgtactttgtcctcccagatgactaccagaattgagattgactctttcgtcgacggtatcgacttctctgagcaactgtctagagctaagtttgaggagatcaacattgaattattcaagaaaacactgaaaccagttgaacaagtcctcaaagacgctggtgtcaagaaatctgaaattgatgacattgtcttggttggtggttctaccagaattccaaaggttcaacaattattggaggattactttgacggaaagaaggcttctaagggaattaacccagatgaagctgtcgcatacggtgctgctgttcaggctggtgttttgtctggtgaggaaggtgtcgatgacatcgtcttgcttgatgtgaaccccctaactctgggtatcgagactactggtggcgttatgactaccttaatcaacagaaacactgctatcccaactaagaaatctcaaattttctccactgctgctgacaaccagccaactgtgttgattcaagtttatgagggtgagagagccttggctaaggacaacaacttgcttggtaaattcgagctgactggtattccaccagctccaagaggtactcctcaagttgaggttacttttgttttagacgctaacggaattttgaaggtgtctgccaccgataagggaactggaaaatccgagtccatcaccatcaacaatgatcgtggtagattgtccaaggaggaggttgaccgtatggttgaagaggccgagaagtacgccgctgaggatgctgcactaagagaaaagattgaggctagaaacgctctggagaactacgctcattcccttaggaaccaagttactgatgactctgaaaccgggcttggttctaaattggacgaggacgacaaagagacattgacagatgccatcaaagataccctagagttcttggaagataacttcgacaccgcaaccaaggaagaattagacgaacaaagagaaaagctttccaagattgcttacccaatcacttctaagctatacggtgctccagagggtggtactccacctggtggtcaaggttttgacgatgatgatggagactttgactacgactatgactatgatcatgatgagttgtaagcttggagaccaatgac

SEQ ID NO: 72 - DNA, 35 bp, synthetic construct
  misc_feature 1..35: note = oligonucleotide primer PP7435_Chr2-1167
  source 1..35: mol_type = other DNA
gatctaggtctcccatgctgtcgttaaaaccatct

SEQ ID NO: 73 - DNA, 45 bp, synthetic construct
  misc_feature 1..45: note = oligonucleotide primer PP7435_Chr2-1167_reverse
  source 1..45: mol_type = other DNA
gtcattggtctccaagcttacaactcatcatgatcatagtcatag

SEQ ID NO: 74 - DNA, 949 bp, Pichia pastoris
  source 1..949: mol_type = genomic DNA
gatctaggtctcacatgcccgtagattcttctcataagacagctagcccacttccacctcgtaaaagagcaaagacggaagaagaaaaggagcagcgtcgagtggaacgtatcctacgtaataggagagcggcccatgcttccagagagaagaaacgtagacacgttgaatttctggaaaaccacgtcgtcgacctggaatctgcacttcaagaatcagccaaagccactaacaagttgaaagaaatacaagatatcattgtttcaaggttggaagccttaggtggtaccgtctcagatttggatttaacagttccggaagtcgattttcccaaatcttctgatttggaacccatgtctgatctctcaacttcttcgaaatcggagaaagcatctacatccactcgcagatctttgactgaggatctggacgaagatgacgtcgctgaatatgacgacgaagaagaggacgaagagttacccaggaaaatgaaagtcttaaacgacaaaaacaagagcacatctatcaagcaggagaagttgaatgaacttccatctcctttgtcatccgatttttcagacgtagatgaagaaaagtcaactctcacacatttaaagttgcaacagcaacaacaacaaccagtagacaattatgtttctactcctttgagtctgccggaggattcagttgattttattaacccaggtaacttaaaaatagagtccgatgagaacttcttgttgagttcaaatactttacaaataaaacacgaaaatgacaccgactacattactacagctccatcaggttccatcaatgatttttttaattcttatgacattagcgagtcgaatcggttgcatcatccagcagcaccatttaccgctaatgcatttgatttaaatgactttgtattcttccaggaatagtaggcttcgagaccaatgac

SEQ ID NO: 75 - DNA, 33 bp, synthetic construct
  misc_feature 1..33: note = oligonucleotide primer PP7435_Chr1-0700
  source 1..33: mol_type = other DNA
gatctaggtctcacatgcccgtagattcttctc

SEQ ID NO: 76 - DNA, 42 bp, synthetic construct
  misc_feature 1..42: note = oligonucleotide primer PP7435_Chr1-0700_reverse
  source 1..42: mol_type = other DNA
gtcattggtctcgaagcctactattcctggaagaatacaaag

SEQ ID NO: 77 - DNA, 2716 bp, Pichia pastoris
  source 1..2716: mol_type = genomic DNA
gatctaggtctcccatgagaacacaaaagatagtaacagtactttgtttgctactaaatactgtgcttggagctctgttgggcatcgattatggtcaagagtttactaaggctgtcctagtggctcctggtgtcccttttgaagttatcttgactccagactccaaacgtaaagataattcaatgatggccatcaaggaaaattccaaaggtgaaattgagagatattatggatcctcagctagttctgtttgtatcagaaaccctgaaacttgcttgaatcatctgaagtcattgataggtgtttcaattgatgacgtttcaactatagattacaagaagtaccattcaggtgctgagatggttccatccaaaaataacaggaacacggttgcctttaagttgggctcttctgtatatcctgtagaagagatacttgctatgagtttagatgacattaaatctagagctgaagatcatttaaaacacgcggtgccaggttcctattcagttatcagtgatgctgtcatcacagtacccactttttttacccaatcgcaaagactggccttgaaagatgctgccgaaattagtggcttaaaagtcgttggcttggttgatgacggtatatctgtggccgttaactatgcctcttcaaggcagttcaatggagacaaacaatatcatatgatctatgacatgggggctggttctttacaggcgactttggtttctatatcttccagtgatgatggtggaattgttattgatgtagaggctattgcctatgacaagtcgctgggaggccagttgttcacacaatctgtttatgacatccttttgcagaagttcttgtctgagcatccttcctttagcgagtccgacttcaacaagaatagtaaatctatgtcaaaactttggcaagcggctgaaaaggcaaagacaattttgagtgcaaacactgacacaagagtttccgttgaatccttatacaatgacattgactttagagccacaatagcaagagacgaattcgaagattacaatgcagagcatgttcataggatcactgctcctatcatcgaggccttaagtcatccattgaatgggaatctgacgtcaccttttccactgaccagtttaagttcagtaattctcacaggcgggtcaacaagagtgccgatggtgaaaaagcacctagaatctttgctaggatctgaattgattgcaaagaatgttaacgctgatgagtcagccgtttttggttctactctccgtggtgtaactttatcgcaaatgttcaaagcgaaacagatgaccgtaaatgaaagaagtgtatatgactattgcctaaaagttggttcttcagagataaacgtgttcccagttggcacccctcttgctactaagaaagtggtcgagctggaaaatgtagacagtgagaaccagctcacgattgggctctacgagaacggacaattgtttgccagtcatgaggttacagacctcaagaagagtatcaaatctctaactcaagaaggtaaagagtgttctaatattaattacgaggctacagtcgagttatctgagagcagattgctttctttaactcgtctgcaggccaaatgtgctgacgaggctgaatatttacctcctgtggacacagagtctgaggatactaaatctgaaaactcaactactagtgagactattgaaaaaccaaacaagaagctattctatcctgtgactatacctactcaactgaaatccgttcacgtgaaaccaatggggtcctctaccaaggtatcttcatctttgaaaatcaaggagttgaacaagaaggatgctgtaaagagatcgatcgaagaattgaagaatcagctggaatcgaaattataccgcgtgcgctcgtatttagaggatgaggaagtggttgaaaaagggccagcatcacaagttgaggctttgtcaacactggttgctgagaatcttgagtggttggactatgatagcgacgatgcatcagcaaaagatatcagggaaaaactaaattctgtgtcagatagtgttgccttcatcaagagctacattgatctgaacgatgtcacttttgataataatcttttcactacgatttacaacactactttaaactccatgcaaaatgttcaagaactaatgttaaacatgagtgaggatgctctgagtttaatgcagcagtatgagaaggaaggtttagacttcgccaaagaaagtcaaaagatcaaaataaaatctcctcctttatcagacaaagagcttgataatctctttaacactgttaccgaaaagttagagcatgtcagaatgttgactgaaaaggacactataagtgatttgcctagagaggagctttttaagctgtatcaagaattgcagaactactcttcccgatttgaagcaatcatggccagtttggaagatgtacactctcaaagaatcaaccgtttgacagacaagttacgcaaacatattgaaagggtgagcaatgaagcattgaaggcagctctcaaggaagctaaacgtcaacaagaggaggaaaaaagccacgagcagaatgagggagaagagcaaagttctgcttccacttctcacactaatgaagatatagaggaaccatcagaatcgcctaaggttcaaacatcccatgatgagttgtaagcttggagaccaatgac

SEQ ID NO: 78 - DNA, 42 bp, synthetic construct
  misc_feature 1..42: note = oligonucleotide primer PP7435_Chr1-0059
  source 1..42: mol_type = other DNA
gatctaggtctcccatgagaacacaaaagatagtaacagtac

SEQ ID NO: 79 - DNA, 40 bp, synthetic construct
  misc_feature 1..40: note = oligonucleotide primer PP7435_Chr1-0059_reverse
  source 1..40: mol_type = other DNA
gtcattggtctccaagcttacaactcatcatgggatgttt

SEQ ID NO: 80 - DNA, 1150 bp, Pichia pastoris
  source 1..1150: mol_type = genomic DNA
gatctaggtctcccatgaaagtgacattatctgtgttagctattgcctcccaattggttagaatcgtttgttcggaaggagaaaatatctgcataggtgaccagtgctatccgaagaattttgaacctgacaaggagtggaaacctgttcaggaaggccagattatccctccaggatcacacgtaagaatggactttaatacacaccagagagaggcaaaactggtggaagagaatgaggatatagacccctcatcattgggagtggctgtagtggattccaccggttcgtttgctgatgatcaatctttggaaaagattgagggactttccatggaacaactagatgagaagttagaagaactgattgagctttcccatgactacgagtacggatcagacataatcttgagtgatcagtatatttttggagtagccgggctagttcctactaagacaaagtttacttctgagttgaaggaaaaggccttgagaattgtcggatcatgcttgagaaacaatgccgatgcggtagagaaactactgggaactgttccaaatactataaccatacaattcatgtcaaacctagtgggtaaagtaaattccactggagagaatgttgactctgttgaacagaaacgaatcctttcaattattggagctgttattcctttcaaaattggaaaggtattgtttgaagcttgttcgggaacgcagaagctattactatccttggataaactggaaagttcagttcaactgagaggataccaaatgttggacgacttcattcatcaccctgaagaggaacttctctcttcattgacagcaaaggaacgattagtaaagcatattgagttgattcaatcattttttgcatcaggaaagcattctcttgatatagcaataaatcgtgagttattcactaggctgattgccttacgaaccaatttagaatctgccaatccaaatctatgtaaaccatcaactgactttttgaactggctgatcgacgaaattgaagctacgaaagataccgatccacacttttcaaaagagcttaaacatttacgttttgaactttttgggaacccattggcatctaggaaaggtttctccgatgagttataagcttggagaccaatgac

SEQ ID NO: 81 - DNA, 40 bp, synthetic construct
  misc_feature 1..40: note = oligonucleotide primer PP7435_Chr1-0550
  source 1..40: mol_type = other DNA
gatctaggtctcccatgaaagtgacattatctgtgttagc

SEQ ID NO: 82 - DNA, 42 bp, synthetic construct
  misc_feature 1..42: note = oligonucleotide primer PP7435_Chr1-0550_reverse
  source 1..42: mol_type = other DNA
gtcattggtctccaagcttataactcatcggagaaacctttc

SEQ ID NO: 83 - DNA, 931 bp, Pichia pastoris
  source 1..931: mol_type = genomic DNA
gatctaggtctcccatgaaactacaccttgtgattctctgtttgatcactgctgtctactgtttcagtgctgttgacagagaaatctttcagctcaaccatgaattacgccaggaatacggagataattttaatttctatgaatggttgaagcttccaaaaggtccctcgtccacgtttgaagatatcgacaacgcgtacaagaaactatcccgtaagttacaccccgataagataagacagaagaaactatcccaggaacaatttgagcaattgaagaaaaaggctaccgaaagataccaacaattgagtgctgtgggatccatcttaagatccgagagcaaagagcgttacgattattttgtcaaacatggattcccagtctataaaggtaacgattacacctatgccaagtttagaccatccgttttgctcacaattttcatcctttttgcgttagctacgttaacccactttgtctttatcagattgtcggccgtgcaatctagaaaaagactgagttcgttgatagaggagaacaaacagctggcttggccacaaggtgttcaagatgtcactcaagtgaaggacgtcaaagtctataacgaacatctacgtaaatggtttttggtatgtttcgacggatccgttcattatgtggagaacgataaaaccttccatgttgatccggaagaagttgaactcccatcttggcaggacactcttccaggtaaattaatagtcaagctgataccccagcttgctagaaagccacgatctccaaaggagatcaagaaggaaaatttagatgataaaaccagaaagacaaaaaaacctacaggggattccaaaactttacctaacggtaaaaccatttataaagctaccaaatccggtggacgtagaaggaaataagcttggagaccaatgac

SEQ ID NO: 84 - DNA, 38 bp, synthetic construct
  misc_feature 1..38: note = oligonucleotide primer PP7435_Chr1-0136
  source 1..38: mol_type = other DNA
gatctaggtctcccatgaaactacaccttgtgattctc

SEQ ID NO: 85 - DNA, 38 bp, synthetic construct
  misc_feature 1..38: note = oligonucleotide primer PP7435_Chr1-0136_reverse
  source 1..38: mol_type = other DNA
gtcattggtctccaagcttatttccttctacgtccacc

SEQ ID NO: 86 - DNA, 918 bp, synthetic construct
  source 1..918: mol_type = other DNA
atgcctgtcgattcttctcacaagaccgctagtccactaccacctagaaagagagcaaagactgaagaggaaaaggaacagagaagagtcgaacgtattctcaggaatagacgtgctgcccacgcttcaagagagaaaaagcgtagacacgtcgagttcctagagaatcatgttgtggacctggaatcggccctccaagagagcgctaaggcaaccaacaagcttaaagagattcaagacattattgtcagcagactagaagctctaggaggtactgtctccgatttggacctgactgttccagaagtagactttcccaagtcctctgatttggagccaatgtcagacctttctacttcttccaagtcggaaaaggcttccacctctactagaagatcccttacagaggacctggacgaggacgacgttgctgaatacgatgacgaggaggaagatgaggaactgcctagaaaaatgaaagttctcaacgacaaaaacaaatctacctctatcaagcaagagaagcttaacgagctgccatctccactaagttctgatttttccgacgttgacgaagagaagtcaaccctaacccacctcaagttacaacagcaacaacaacagcctgttgacaactatgtttctacgcctctgtctttgccagaagattctgtggatttcattaacccaggtaacctcaagatcgaatccgacgaaaacttcctgctttcctctaacacacttcaaatcaagcacgagaacgatactgactacattactactgctccttctggttctatcaacgactttttcaattcatacgacatctccgaatctaacagactacatcaccctgccgctccatttaccgcaaacgccttcgatctcaatgacttcgttttcttccaggagtaatag

SEQ ID NO: 87 - DNA, 918 bp, synthetic construct
  source 1..918: mol_type = other DNA
atgccagttgatagttcgcacaagactgcttctccactgccacctagaaagagagctaagactgaggaggaaaaggagcaacgtagagtcgagagaatcctgagaaaccgtagagccgctcacgcctctagagagaaaaagagaaggcatgttgaatttcttgaaaaccacgtcgtcgatctcgaatctgcccttcaagagtcagctaaagctaccaacaagctaaaggaaattcaagacattatcgtatctagactggaggcacttggtggtactgtttctgacctggatcttacagttccagaagttgacttcccaaaatccagtgatctagaacctatgtctgatctatctacctcaagcaagtctgagaaggcaagcacgtcaaccagacgttccctaactgaggacctggacgaagatgatgtcgctgaatacgatgacgaggaggaggatgaggaactgcctagaaaaatgaaggttcttaacgacaaaaacaagtctacctctatcaaacaggaaaagctcaacgaactcccatcccctctctcttccgacttctccgacgtggacgaggaaaagtctactttgacccacctgaagttgcaacaacaacagcaacaacctgttgacaactatgtctccactcctctctcactcccagaggactcggttgacttcatcaaccccggtaaccttaagattgaatctgacgagaacttccttctatcctctaataccttacagattaagcatgaaaatgatactgactacattactaccgctccatccggatctatcaatgacttcttcaattcttacgacatttctgagtccaacagattgcaccacccagctgcaccttttacagccaacgcttttgacctaaacgacttcgtgtttttccaggagtaatag

SEQ ID NO: 88 - DNA, 918 bp, synthetic construct
  source 1..918: mol_type = other DNA
atgcctgtagattcctcccacaagactgcttcgccacttcctcctcgtaagagagctaagaccgaagaggaaaaggaacagagaagagttgagagaatcctgagaaacagaagggctgctcacgccagtagagagaaaaagcgtcgtcacgtcgagttcctagaaaatcatgttgttgatttggaatcagccctgcaagagtctgccaaagcaaccaacaagctgaaggagatccaggacattattgtctctagacttgaagctctaggtggtactgtctctgacctagacctcaccgtgcctgaggttgactttccaaagtcgtctgatctcgaaccaatgtctgacctatcaacttcatctaagtctgagaaggcaagcacctctaccagaagatcccttacagaggacctcgatgaagatgacgttgctgaatatgatgacgaagaggaggacgaggaattacctagaaagatgaaggtcttgaacgacaaaaacaaatccacttctattaagcaggaaaagctaaacgagctgccatctccactctcctctgatttcagcgacgtggacgaggagaaatctacactcactcaccttaagctacaacaacagcaacaacaacccgttgacaactacgtttccaccccactttctctcccagaggacagtgttgatttcattaaccctggaaacctgaaaatcgagtcagacgaaaacttcctactgtcctccaacacgcttcaaatcaagcacgaaaatgacactgattacattactactgctccatctggttctattaacgactttttcaattcctacgatatttctgaatctaacagattgcaccatcctgctgccccattcaccgcaaacgcttttgacctcaatgacttcgtctttttccaagagtaatag